(19) United States
(12) Patent Application Publication
(10) Pub. No.: US 2020/0027096 A1
(43) Pub. Date: Jan. 23, 2020

(54) SYSTEM, BUSINESS AND TECHNICAL METHODS, AND ARTICLE OF MANUFACTURE FOR UTILIZING INTERNET OF THINGS TECHNOLOGY IN ENERGY MANAGEMENT SYSTEMS DESIGNED TO AUTOMATE THE PROCESS OF GENERATING AND/OR MONETIZING CARBON CREDITS

(71) Applicant: JASON RYAN COONER, PINSON, AL (US)

(72) Inventor: JASON RYAN COONER, PINSON, AL (US)

(21) Appl. No.: 16/181,215

(22) Filed: Nov. 5, 2018

Related U.S. Application Data
(60) Provisional application No. 62/582,918, filed on Nov. 7, 2017.

(51) Int. Cl. G06Q 30/00; H04L 29/08; G06Q 20/38; G06Q 40/04; G01N 33/00

(52) U.S. Cl. CPC G06Q 30/018; H04L 67/12; G01W 1/00; G06Q 40/04; G01N 33/0063; G06Q 20/389

(57) ABSTRACT

A carbon credit is a generic term for any tradable certificate or permit representing the right to emit one ton of carbon dioxide or the mass of another greenhouse gas with a carbon dioxide equivalent (tCO2e) equivalent to one ton of carbon dioxide.

Carbon credits and carbon markets are a component of national and international attempts to mitigate the growth in concentrations of greenhouse gases (GHGs). One carbon credit is equal to one ton of carbon dioxide, or in some markets, carbon dioxide equivalent gases. Carbon trading is an application of an emissions trading approach. Greenhouse gas emissions are capped and then markets are used to allocate the emissions among the group of regulated sources.

Carbon credits can be generated by any process that conforms to ISO 14064-66 standards. Once generated, carbon credits can be stored in a distributed, Cloud-based ledger. The ledger entries can serve as a registry for carbon credits as well as the data source for an Internet-enabled trading system or financial exchange that allows the carbon credits to be sold and bought as part of the same system. The distributed ledger can provide records that combine the details of the carbon credits' origin, transaction history, and financial instructions associated with trading of the carbon credits via a distributed ledger system.

### BACKGROUND

### An Overview of the Internet of Things

### Internet of Things

### From Wikipedia, the Free Encyclopedia

The Internet of things (IoT) is the network of physical devices, vehicles, home appliances, and other items embedded with electronics, software, sensors, actuators, and network connectivity which enable these objects to connect and exchange data. Each thing is uniquely identifiable through its embedded computing system but is able to inter-operate within the existing Internet infrastructure. Experts estimate that the IoT will consist of about 30 billion objects by 2020.

The IoT allows objects to be sensed or controlled remotely across existing network infrastructure, creating opportunities for more direct integration of the physical world into computer-based systems, and resulting in improved efficiency, accuracy and economic benefit in addition to reduced human intervention. When IoT is augmented with sensors and actuators, the technology becomes an instance of the more general class of cyber-physical systems, which also encompasses technologies such as smart grids, virtual power plants, smart homes, intelligent transportation and smart cities.

“Things”, in the IoT sense, can refer to a wide variety of devices such as heart monitoring implants, biochip transponders on farm animals, cameras streaming live feeds of wild animals in coastal waters, automobiles with built-in sensors, DNA analysis devices for environmental/food/pathogen monitoring, or field operation devices that assist firefighters in search and rescue operations. Legal scholars suggest regarding “things” as an “inextricable mixture of hardware, software, data and service”.

These devices collect useful data with the help of various existing technologies and then autonomously flow the data between other devices. The quick expansion of Internet-connected objects is also expected to generate large amounts of data from diverse locations, with the consequent necessity for quick aggregation of the data, and an increase in the need to index, store, and process such data more effectively. In recent years with the massive growth in global cyber threat, there has been a significant rise in exploitation of IoT technologies for committing cyber terror crimes.

The term “the Internet of things” was coined by Kevin Ashton of Procter & Gamble, later MIT's Auto-ID Center, in 1999.

### History

As of 2016, the vision of the Internet of things has evolved due to a convergence of multiple technologies, including ubiquitous wireless communication, real-time analytics, machine learning, commodity sensors, and embedded systems. This means that the traditional fields of embedded systems, wireless sensor networks, control systems, automation (including home and building automation), and others all contribute to enabling the Internet of things.

The concept of a network of smart devices was discussed as early as 1982, with a modified Coke machine at Carnegie Mellon University becoming the first Internet-connected appliance, able to report its inventory and whether newly loaded drinks were cold. Mark Weiser's seminal 1991 paper on ubiquitous computing, “The Computer of the 21st Century”, as well as academic venues such as UbiComp and PerCom produced the contemporary vision of IoT. In 1994 Reza Raji described the concept in IEEE Spectrum as “[moving] small packets of data to a large set of nodes, so as to integrate and automate everything from home appliances to entire factories”. Between 1993 and 1996 several companies proposed solutions like Microsoft's at Work or Novell's NEST. However, only in 1999 did the field start gathering momentum. Bill Joy envisioned Device to Device (D2D) communication as part of his “Six Webs” framework, presented at the World Economic Forum at Davos in 1999.

The concept of the Internet of things became popular in 1999, through the Auto-ID Center at MIT and related market-analysis publications. Radio-frequency identification (RFID) was seen by Kevin Ashton (one of the founders of the original Auto-ID Center) as a prerequisite for the Internet of things at that point. Ashton prefers the phrase “Internet for things.” If all objects and people in daily life were equipped with identifiers, computers could manage and inventory them. Besides using RFID, the tagging of things may be achieved through such technologies as near field communication, barcodes, QR codes and digital watermarking.

In its original interpretation, one of the first consequences of implementing the Internet of things by equipping all objects in the world with minuscule identifying devices or machine-readable identifiers would be to transform daily life. For instance, instant and ceaseless inventory control would become ubiquitous. A person's ability to interact with objects could be altered remotely based on immediate or present needs, in accordance with existing end-user agreements. For example, such technology could grant motion-picture publishers much more control over end-user private devices by remotely enforcing copyright restrictions and digital rights management, so the ability of a customer who bought a Blu-ray disc to watch the movie could become dependent on the copyright holder's decision, similar to Circuit City's failed DIVX.

A significant transformation is to extend “things” from the data generated from devices to objects in the physical space. The thought-model for future interconnection environment was proposed in 2004. The model includes the notion of the ternary universe consists of the physical world, virtual world and mental world and a multi-level reference architecture with the nature and devices at the bottom level followed by the level of the Internet, sensor network, and mobile network, and intelligent human-machine communities at the top level, which supports geographically dispersed users to cooperatively accomplish tasks and solve problems by using the network to actively promote the flow of material, energy, techniques, information, knowledge, and services in this environment. This thought model envisioned the development trend of the Internet of things.

### Applications

### A Nest Learning Thermostat Reporting on Energy Usage and Local Weather.

A 2012 Internet Refrigerator from LG

The applications for internet connected devices are extensive. Multiple categorizations have been suggested, most of which agree on a separation between consumer, enterprise (business), and infrastructure applications. George Osborne, the former British Chancellor of the Exchequer, posited that the Internet of things is the next stage of the information revolution and referenced the inter-connectivity of everything from urban transport to medical devices to household appliances.

The ability to network embedded devices with limited CPU, memory and power resources means that IoT finds applications in nearly every field. Such systems could be in charge of collecting information in settings ranging from natural ecosystems to buildings and factories, thereby finding applications in fields of environmental sensing and urban planning.

Intelligent shopping systems, for example, could monitor specific users' purchasing habits in a store by tracking their specific mobile phones. These users could then be provided with special offers on their favorite products, or even location of items that they need, which their fridge has automatically conveyed to the phone. Additional examples of sensing and actuating are reflected in applications that deal with heat, water, electricity and energy management, as well as cruise-assisting transportation systems. Other applications that the Internet of things can provide is enabling extended home security features and home automation. The concept of an “Internet of living things” has been proposed to describe networks of biological sensors that could use cloud-based analyses to allow users to study DNA or other molecules.

Consumer application A growing portion of IoT devices are created for consumer use. Examples of consumer applications include connected car, entertainment, home automation (also known as smart home devices), wearable technology, quantified self, connected health, and appliances such as washer/dryers, robotic vacuums, air purifiers, ovens, or refrigerators/freezers that use Wi-Fi for remote monitoring. Consumer IoT provides new opportunities for user experience and interfaces.

Some consumer applications have been criticized for their lack of redundancy and their inconsistency, leading to a popular parody known as the “Internet of Shift.” Companies have been criticized for their rush into IoT, creating devices of questionable value and not setting up stringent security standards.

### Smart Home

IoT devices are a part of the larger concept of Home automation, also known as domotics. Large smart home systems utilize a main hub or controller to provide users with a central control for all of their devices.

One application of the smart home is to provide assistance for disabled and elderly individuals. These home systems utilize assistive technology to accommodate an owner's specific disabilities. Voice control can assist users with sight and mobility limitations while alert systems can be connected directly to Cochlear implants worn by hearing impaired users. They can also be equipped with additional safety features. These features can include sensors that monitor for medical emergencies such as falls or seizures. Smart home technology applied in this way can provide users with more freedom and a higher quality of life.

### Enterprise

The term “Enterprise IoT,” or EIoT, is used to refer to all devices used in business and corporate settings. By 2019, it is estimated the EIoT will account for nearly 40% or 9.1 billion devices. Large companies will see a more immediate profit from the widespread automation that IoT devices.

### Media

In order to hone the manner in which things, media and big data are interconnected, it is first necessary to provide some context into the mechanism used for media process. It has been suggested by Nick Couldry and Joseph Turow that practitioners in media approach big data as many actionable points of information about millions of individuals. The industry appears to be moving away from the traditional approach of using specific media environments such as newspapers, magazines, or television shows and instead tap into consumers with technologies that reach targeted people at optimal times in optimal locations. The ultimate aim is, of course, to serve or convey, a message or content that is (statistically speaking) in line with the consumer's mindset. For example, publishing environments are increasingly tailoring the messages (articles) to appeal to consumers that have been exclusively gleaned through various data-mining activities.

The media industries process big data in a dual, interconnected manner:
Targeting of consumers (for advertising by marketers)
Data-capture

Thus, the Internet of things creates an opportunity to measure, collect and analyze an ever-increasing variety of behavioral statistics. Cross-correlation of this data could revolutionize the targeted marketing of products and services. For example, as noted by Danny Meadows-Klue, the combination of analytics for conversion tracking with behavioral targeting has unlocked a new level of precision that enables display advertising to be focused on the devices of people with relevant interests. Big data and the IoT work in conjunction. From a media perspective, data is the key derivative of device interconnectivity, whilst being pivotal in allowing clearer accuracy in targeting. The Internet of things, therefore, transforms the media industry, companies and even governments, opening up a new era of economic growth and competitiveness. The wealth of data generated by this industry will allow practitioners in advertising and media to gain an elaborate layer on the present targeting mechanisms used by the industry.

### Infrastructure Management

Monitoring and controlling operations of urban and rural infrastructures like bridges, railway tracks, on- and offshore-wind-farms is a key application of the IoT. The IoT infrastructure can be used for monitoring any events or changes in structural conditions that can compromise safety and increase risk. It can also be used for scheduling repair and maintenance activities in an efficient manner, by coordinating tasks between different service providers and users of these facilities. IoT devices can also be used to control critical infrastructure like bridges to provide access to ships. Usage of IoT devices for monitoring and operating infrastructure is likely to improve incident management and emergency response coordination, and quality of service, up-times and reduce costs of operation in all infrastructure related areas. Even areas such as waste management can benefit from automation and optimization that could be brought in by the IoT.

### Manufacturing

Network control and management of manufacturing equipment, asset and situation management, or manufacturing process control bring the IoT within the realm of industrial applications and smart manufacturing as well. The IoT intelligent systems enable rapid manufacturing of new products, dynamic response to product demands, and real-time optimization of manufacturing production and supply chain networks, by networking machinery, sensors and control systems together.

Digital control systems to automate process controls, operator tools and service information systems to optimize plant safety and security are within the purview of the IoT. But it also extends itself to asset management via predictive maintenance, statistical evaluation, and measurements to maximize reliability. Smart industrial management systems can also be integrated with the Smart Grid, thereby enabling real-time energy optimization. Measurements, automated controls, plant optimization, health and safety management, and other functions are provided by a large number of networked sensors.

The term industrial Internet of things (IIoT) is often encountered in the manufacturing industries, referring to the industrial subset of the IoT. IIoT in manufacturing could generate so much business value that it will eventually lead to the fourth industrial revolution, so the so-called Industry 4.0. It is estimated that in the future, successful companies will be able to increase their revenue through Internet of things by creating new business models and improve productivity, exploit analytics for innovation, and transform workforce. The potential of growth by implementing IIoT will generate $12 trillion of global GDP by 2030

### Design Architecture of Cyber-Physical Systems-Enabled Manufacturing System

While connectivity and data acquisition are imperative for IIoT, they should not be the purpose, rather the foundation and path to something bigger. Among all the technologies, predictive maintenance is probably a relatively “easier win” since it is applicable to existing assets and management systems. The objective of intelligent maintenance systems is to reduce unexpected downtime and increase productivity. And to realize that alone would generate around up to 30% over the total maintenance costs. Industrial big data analytics will play a vital role in manufacturing asset predictive maintenance, although that is not the only capability of industrial big data. Cyber-physical systems (CPS) is the core technology of industrial big data and it will be an interface between human and the cyber world. Cyber-physical systems can be designed by following the 5C (connection, conversion, cyber, cognition, configuration) architecture, and it will transform the collected data into actionable information, and eventually interfere with the physical assets to optimize processes.

### SUMMARY OF THE INVENTION

An IoT-enabled intelligent system of such cases was proposed in 2001 and later demonstrated in 2014 by the National Science Foundation Industry/University Collaborative Research Center for Intelligent Maintenance Systems (IMS) at the University of Cincinnati on a band saw machine in IMTS 2014 in Chicago. Band saw machines are not necessarily expensive, but the band saw belt expenses are enormous since they degrade much faster. However, without sensing and intelligent analytics, it can be only determined by experience when the band saw belt will actually break. The developed prognostics system will be able to recognize and monitor the degradation of band saw belts even if the condition is changing, advising users when is the best time to replace band saw. This will significantly improve user experience and operator safety and ultimately save on costs.

### Agriculture

The IoT contributes significantly towards innovating farming methods. Farming challenges caused by population growth and climate change have made it one of the first industries to utilize the IoT. The integration of wireless sensors with agricultural mobile apps and cloud platforms helps in collecting vital information pertaining to the environmental conditions—temperature, rainfall, humidity, wind speed, pest infestation, soil humus content or nutrients, besides others—linked with a farmland, can be used to improve and automate farming techniques, take informed decisions to improve quality and quantity, and minimize risks and wastes. The app-based field or crop monitoring also lowers the hassles of managing crops at multiple locations. For example, farmers can now detect which areas have been fertilized (or mistakenly missed), if the land is too dry and predict future yields.

### Energy Management

Integration of sensing and actuation systems, connected to the Internet, is likely to optimize energy consumption as a whole. It is expected that IoT devices will be integrated into all forms of energy consuming devices (switches, power outlets, bulbs, televisions, etc.) and be able to communicate with the utility supply company in order to effectively balance power generation and energy usage. Such devices would also offer the opportunity for users to remotely control their devices, or centrally manage them via a cloud-based interface, and enable advanced functions like scheduling (e.g., remotely powering on or off heating systems, controlling ovens, changing lighting conditions etc.).

Besides home-based energy management, the IoT is especially relevant to the Smart Grid since it provides systems to gather and act on energy and power-related information in an automated fashion with the goal to improve the efficiency, reliability, economics, and sustainability of the production and distribution of electricity. Using advanced metering infrastructure (AMI) devices connected to the Internet backbone, electric utilities can not only collect data from end-user connections but also, manage other distribution automation devices like transformers and reclosers.

### Environmental Monitoring

Environmental monitoring applications of the IoT typically use sensors to assist in environmental protection by monitoring air or water quality, atmospheric or soil conditions, and can even include areas like monitoring the movements of wildlife and their habitats. Development of resource-constrained devices connected to the Internet also means that other applications like earthquake or tsunami early-warning systems can also be used by emergency services to provide more effective aid. IoT devices in this application typically span a large geographic area and can also be mobile. It has been argued that the standardization IoT brings to wireless sensing will revolutionize this area.

### Building and Home Automation

IoT devices can be used to monitor and control the mechanical, electrical and electronic systems used in various types of buildings (e.g., public and private, industrial, institutions, or residential) in home automation and building automation systems. In this context, three main areas are being covered in literature:
The integration of the internet with building energy management systems in order to create energy efficient and IOT driven “smart buildings”.
The possible means of real-time monitoring for reducing energy consumption and monitoring occupant behaviors.
The integration of smart devices in the built environment and how they might be used in future applications.

### Metropolitan Scale Deployments

There are several planned or ongoing large-scale deployments of the IoT, to enable better management of cities and systems. For example, Songdo, South Korea, the first of its kind fully equipped and wired smart city, is on near completion. Nearly everything in this city is planned to be wired, connected and turned into a constant stream of data that would be monitored and analyzed by an array of computers with little, or no human intervention.

Another application is a currently undergoing project in Santander, Spain. For this deployment, two approaches have been adopted. This city of 180,000 inhabitants, has already seen 18,000 city application downloads for their smartphones. This application is connected to 10,000 sensors that enable services like parking search, environmental monitoring, digital city agenda among others. City context information is used in this deployment so as to benefit merchants through a spark deals mechanism based on city behavior that aims at maximizing the impact of each notification.

Other examples of large-scale deployments underway include the Sino-Singapore Guangzhou Knowledge City; work on improving air and water quality, reducing noise pollution, and increasing transportation efficiency in San Jose, Calif.; and smart traffic management in western Singapore. French company, Sigfox, commenced building an ultra-narrowband wireless data network in the San Francisco Bay Area in 2014, the first business to achieve such a deployment in the U.S. It subsequently announced it would set up a total of 4000 base stations to cover a total of 30 cities in the U.S. by the end of 2016, making it the largest IoT network coverage provider in the country thus far.

Another example of a large deployment is the one completed by New York Waterways in New York City to connect all the city's vessels and be able to monitor them live 24/7. The network was designed and engineered by Fluidmesh Networks, a Chicago-based company developing wireless networks for critical applications. The NYWW network is currently providing coverage on the Hudson River, East River, and Upper New York Bay. With the wireless network in place, NY Waterway is able to take control of its fleet and passengers in a way that was not previously possible. New applications can include security, energy and fleet management, digital signage, public Wi-Fi, paperless ticketing and others.

### Other Fields of Application

### Medical and Healthcare

IoT devices can be used to enable remote health monitoring and emergency notification systems. These health monitoring devices can range from blood pressure and heart rate monitors to advanced devices capable of monitoring specialized implants, such as pacemakers, Fitbit electronic wristbands, or advanced hearing aids. Some hospitals have begun implementing “smart beds” that can detect when they are occupied and when a patient is attempting to get up. It can also adjust itself to ensure appropriate pressure and support is applied to the patient without the manual interaction of nurses.

Specialized sensors can also be equipped within living spaces to monitor the health and general well-being of senior citizens, while also ensuring that proper treatment is being administered and assisting people regain lost mobility via therapy as well. Other consumer devices to encourage healthy living, such as, connected scales or wearable heart monitors, are also a possibility with the IoT. More and more end-to-end health monitoring IoT platforms are coming up for antenatal and chronic patients, helping one manage health vitals and recurring medication requirements.

The Research & Development Corporation (DEKA), a company that creates prosthetic limbs, has created a battery-powered arm that uses myoelectricity, a device that converts muscle group sensations into motor control. The arm is nicked named Luke Arm after Luke Skywalker (Star Wars).

### Transportation

### Digital Variable Speed-Limit Sign.

The IoT can assist in the integration of communications, control, and information processing across various transportation systems. Application of the IoT extends to all aspects of transportation systems (i.e. the vehicle, the infrastructure, and the driver or user).

Dynamic interaction between these components of a transport system enables inter and intra vehicular communication, smart traffic control, smart parking, electronic toll collection systems, logistic and fleet management, vehicle control, and safety and road assistance. In Logistics and Fleet Management for example, The IoT platform can continuously monitor the location and conditions of cargo and assets via wireless sensors and send specific alerts when management exceptions occur (delays, damages, thefts, etc.).

### Trends and Characteristics

### Technology Roadmap: Internet of Things.

The interconnection via the Internet of computing devices embedded in everyday objects, enabling them to send and receive data.

### Intelligence

Ambient intelligence and autonomous control are not part of the original concept of the Internet of things. Ambient intelligence and autonomous control do not necessarily require Internet structures, either. However, there is a shift in research to integrate the concepts of the Internet of things and autonomous control, with initial outcomes towards this direction considering objects as the driving force for autonomous IoT.

### BRIEF DESCRIPTION OF THE DRAWINGS

FIG. 1. POTENTIAL INTERNET OF THINGS (IOT) EDGE HARDWARE LAYOUT INCLUDING SENSOR DEVICES, EDGE ROUTERS, AND EDGE GATEWAYS WITH BLUETOOTH, ZIGBEE, WIFI, ZWAVE, SUB-GIGAHERTZ, CELLULAR, SATELLITE, LORAWAN, SIGFOX, OR ALTERNATE WIRELESS COMMUNICATIONS TECHNOLOGY PROVIDED FOR NETWORK ACCESS

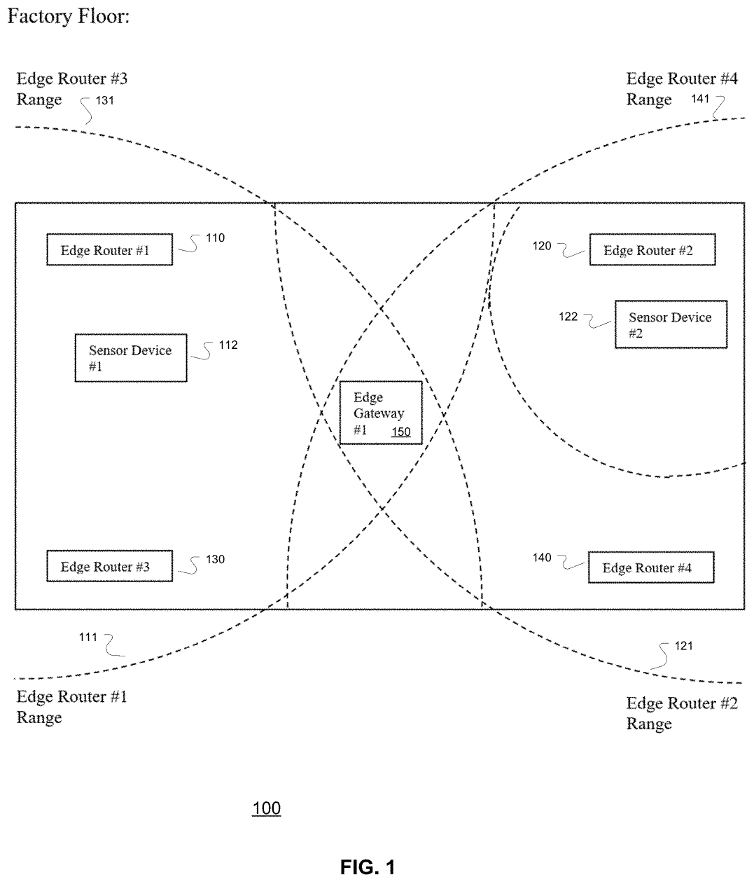

FIG. 2. ALTERNATIVE INTERNET OF THINGS (IOT) EDGE HARDWARE LAYOUT INCLUDING SENSOR DEVICES, EDGE ROUTERS, AND EDGE GATEWAYS WITH BLUETOOTH, ZIGBEE, WIFI, ZWAVE, SUB-GIGAHERTZ, CELLULAR, SATELLITE, LORAWAN, SIGFOX, OR ALTERNATE WIRELESS COMMUNICATIONS TECHNOLOGY PROVIDED FOR NETWORK ACCESS

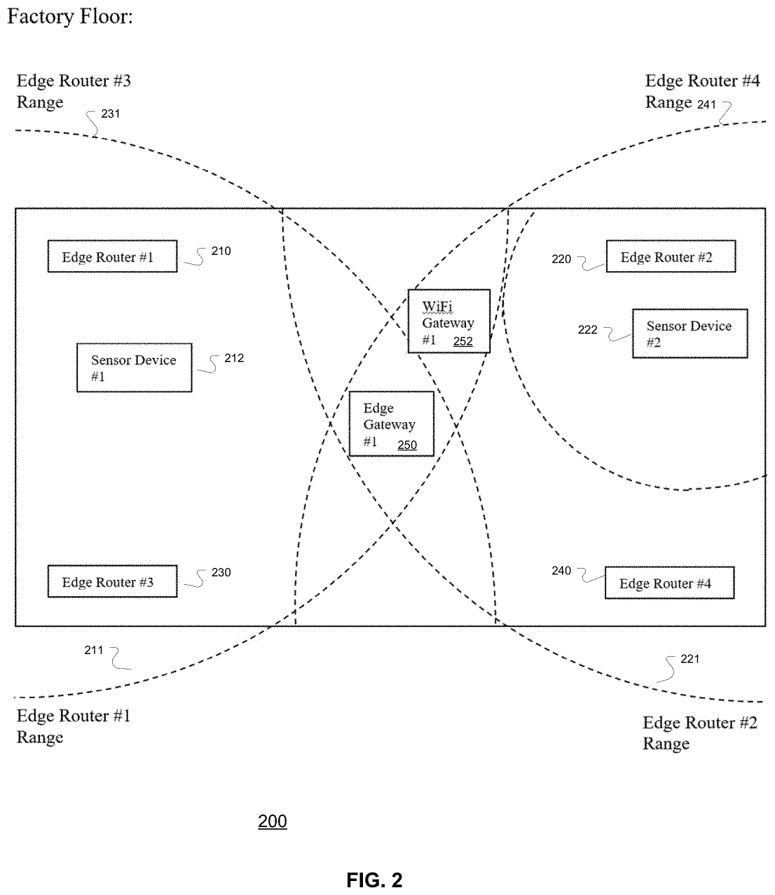

FIG. 3. SYSTEM ARCHITECTURE FOR IOT BASED ENERGY EFFICIENCY MONITORING WITH CARBON CREDIT GENERATION AND VALIDATION

FIG. 4. ISO 14064-3 VALIDATION AND VERIFICATION PROCESS

FIG. 5: COMPONENTS OF A GHG INFORMATION MANAGEMENT SYSTEM

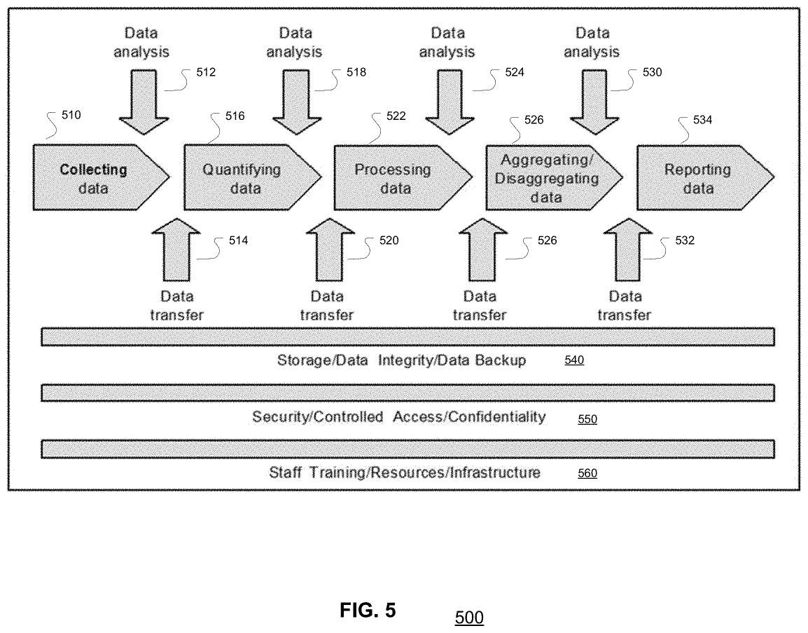

FIG. 6: ROLES AND RESPONSIBILITIES IN A GHG MANAGEMENT SYSTEM

FIG. 7: VALIDATION AND VERIFICATION ACTIVITIES AND DECISION POINTS

FIG. 8: GUIDANCE ON GHG INFORMATION SAMPLE DESIGN

FIG. 9: EXAMPLES OF ERROR CHECKING TESTS AND CONTROLS

FIG. 10: RELATIONSHIP BETWEEN GHG INFORMATION TYPES/SOURCES TO RELATIVE ACCURACY

FIG. 11: TYPICAL INFORMATION TO REVIEW IN VERIFYING GHG EMISSIONS AND REMOVALS ESTIMATES

FIG. 12: RELATIONSHIP OF THE THREE PARTS OF ISO 14064, AND THEIR RELATIONSHIP TO ISO 14065 AND ISO 14066

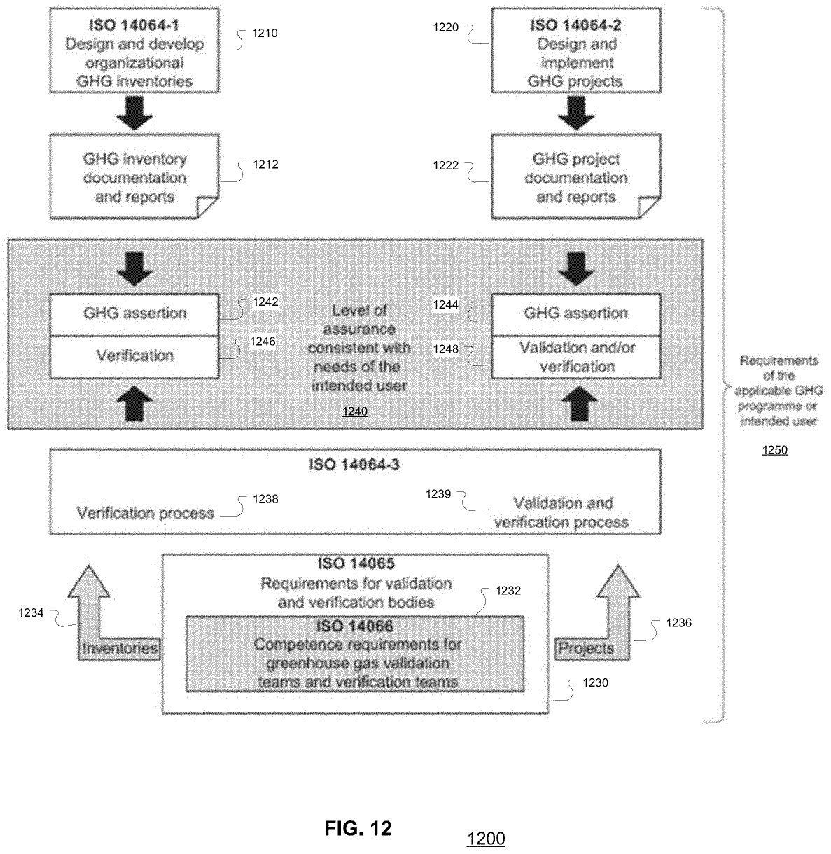

FIG. 13: ALTERNATE ISO 14064-3 VALIDATION AND VERIFICATION PROCESS

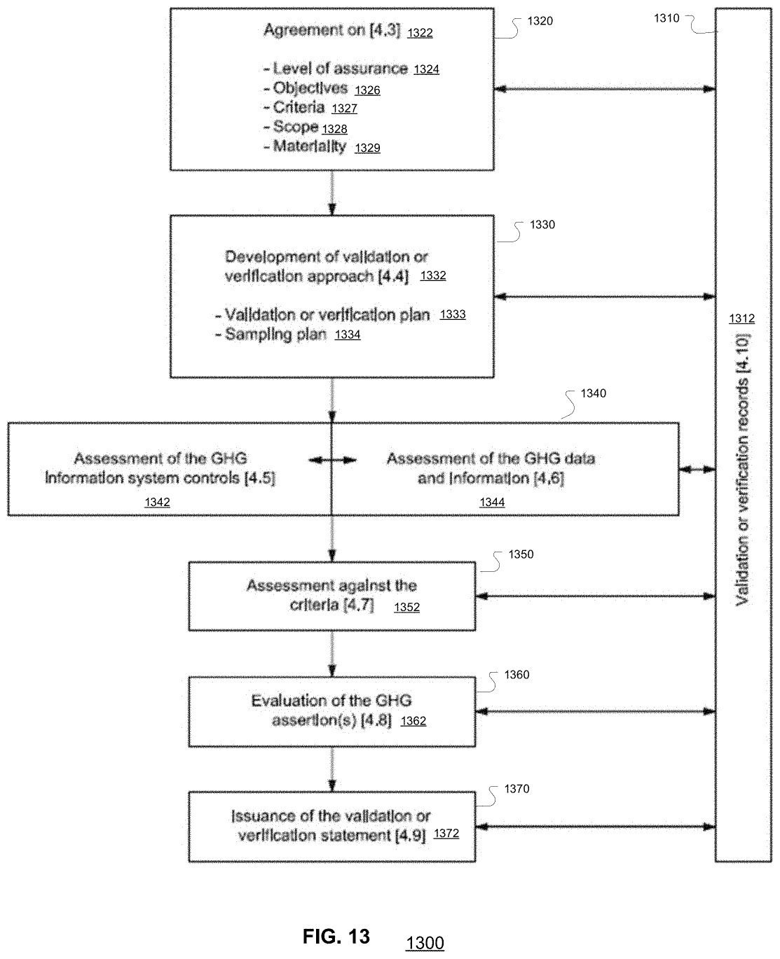

FIG. 14: POTENTIAL COMPONENTS OF A GHG PROGRAM

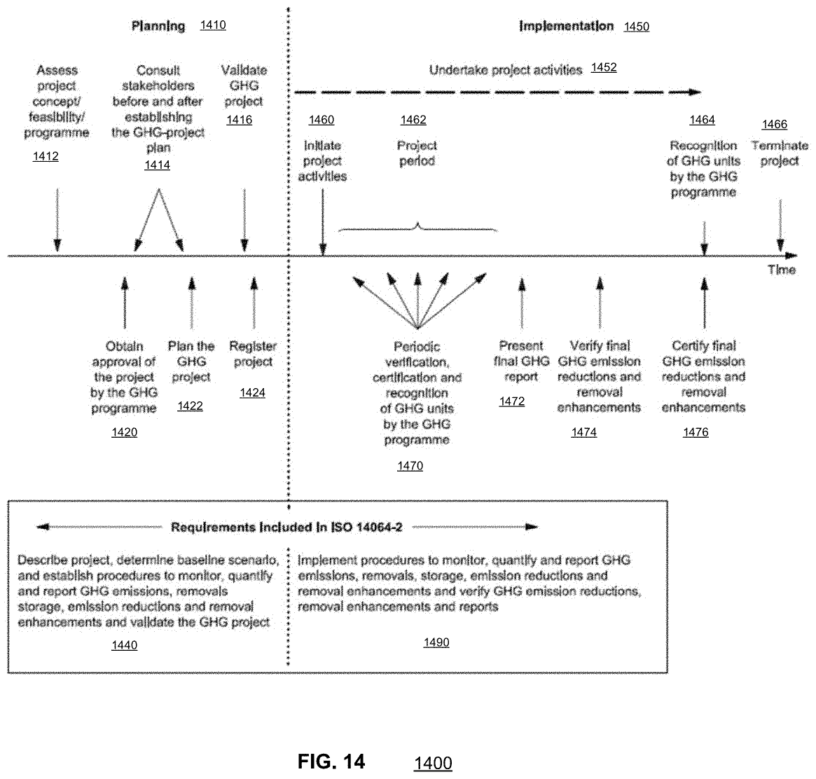

FIG. 15: OVERALL WORKFLOW FOR VALIDATION AND/OR VERIFICATION OF A GHG PROGRAM PER ISO 14064-66 STANDARDS

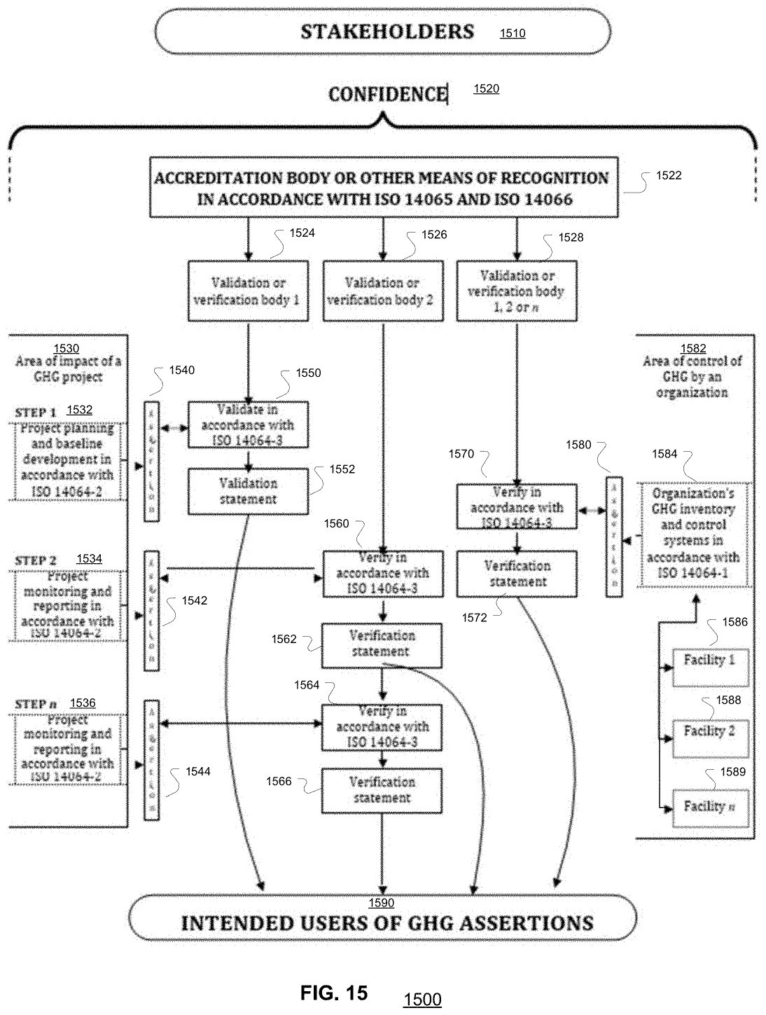

### DETAILED DESCRIPTION

In the future, the Internet of things may be a non-deterministic and open network in which auto-organized or intelligent entities (Web services, SOA components), virtual objects (avatars) will be interoperable and able to act independently (pursuing their own objectives or shared ones) depending on the context, circumstances or environments. Autonomous behavior through the collection and reasoning of context information as well as the objects ability to detect changes in the environment, faults affecting sensors and introduce suitable mitigation measures constitute a major research trend, clearly needed to provide credibility to the IoT technology. Modern IoT products and solutions in the marketplace use a variety of different technologies to support such context-aware automation but more sophisticated forms of intelligence are requested to permit sensor units to be deployed in real environments.

### Architecture

The system will likely be an example of event-driven architecture, bottom-up made (based on the context of processes and operations, in real-time) and will consider any subsidiary level. Therefore, model driven and functional approaches will coexist with new ones able to treat exceptions and unusual evolution of processes (multi-agent systems, B-ADSc, etc.).

In an Internet of Things, the meaning of an event will not necessarily be based on a deterministic or syntactic model but would instead be based on the context of the event itself: this will also be a semantic web. Consequently, it will not necessarily need common standards that would not be able to address every context or use: some actors (services, components, avatars) will accordingly be self-referenced and, if ever needed, adaptive to existing common standards (predicting everything would be no more than defining a “global finality” for everything that is just not possible with any of the current top-down approaches and standardizations).

Building on top of the Internet of things, the web of things is an architecture for the application layer of the Internet of things looking at the convergence of data from IoT devices into Web applications to create innovative use-cases. In order to program and control the flow of information in the Internet of things, a predicted architectural direction is being called BPM Everywhere which is a blending of traditional process management with process mining and special capabilities to automate the control of large numbers of coordinated devices.

### Network Architecture

The Internet of things requires huge scalability in the network space to handle the surge of devices. IETF 6LoWPAN would be used to connect devices to IP networks. With billions of devices being added to the Internet space, IPv6 will play a major role in handling the network layer scalability. IETF's Constrained Application Protocol, ZeroMQ, and MQTT would provide lightweight data transport. “MQ” in “MQTT” came from IBM's MQ Series message queuing product line.

Fog computing is a viable alternative to prevent such large burst of data flow through Internet. The edge devices' computation power can be used to analyse and process data, thus providing easy real time scalability.

### Complexity

In semi-open or closed loops (i.e. value chains, whenever a global finality can be settled) IoT will often be considered and studied as a complex system due to the huge number of different links, interactions between autonomous actors, and its capacity to integrate new actors. At the overall stage (full open loop) it will likely be seen as a chaotic environment (since systems always have finality). As a practical approach, not all elements in the Internet of things run in a global, public space. Subsystems are often implemented to mitigate the risks of privacy, control and reliability. For example, Domestic Robotics (Domotics) running inside a smart home might only share data within and be available via a local network.

### Size Considerations

The Internet of things would encode 50 to 100 trillion objects, and be able to follow the movement of those objects. Human beings in surveyed urban environments are each surrounded by 1000 to 5000 trackable objects.

### Space Considerations

In the Internet of things, the precise geographic location of a thing—and also the precise geographic dimensions of a thing—will be critical. Therefore, facts about a thing, such as its location in time and space, have been less critical to track because the person processing the information can decide whether or not that information was important to the action being taken, and if so, add the missing information (or decide to not take the action). (Note that some things in the Internet of things will be sensors, and sensor location is usually important.) The GeoWeb and Digital Earth are promising applications that become possible when things can become organized and connected by location. However, the challenges that remain include the constraints of variable spatial scales, the need to handle massive amounts of data, and an indexing for fast search and neighbor operations. In the Internet of things, if things are able to take actions on their own initiative, this human-centric mediation role is eliminated. Thus, the time-space context that we as humans take for granted must be given a central role in this information ecosystem. Just as standards play a key role in the Internet and the Web, geospatial standards will play a key role in the Internet of things.

### A Solution to “Basket of Remotes”

Many IoT devices have a potential to take a piece of this market. Jean-Louis Gassée (Apple initial alumni team, and BeOS co-founder) has addressed this topic in an article on Monday Note, where he predicts that the most likely problem will be what he calls the “basket of remotes” problem, where we'll have hundreds of applications to interface with hundreds of devices that don't share protocols for speaking with one another.

There are multiple approaches to solve this problem, one of them called the “predictive interaction”, where cloud or fog based decision makers will predict the user's next action and trigger some reaction.

For user interaction, new technology leaders are joining forces to create standards for communication between devices. Manufacturers are becoming more conscious of this problem, and many companies have begun releasing their devices with open APIs. Many of these APIs are used by smaller companies looking to take advantage of quick integration.

### Frameworks

IoT frameworks might help support the interaction between “things” and allow for more complex structures like distributed computing and the development of distributed applications. Currently, some IoT frameworks seem to focus on real-time data logging solutions, offering some basis to work with many “things” and have them interact. Future developments might lead to specific software-development environments to create the software to work with the hardware used in the Internet of things. Companies are developing technology platforms to provide this type of functionality for the Internet of things. Newer platforms are being developed, which add more intelligence.

REST is a scalable architecture that allows things to communicate over Hypertext Transfer Protocol and is easily adopted for IoT applications to provide communication from a thing to a central web server.

### Enabling Technologies for IoT

There are many technologies that enable IoT. Crucial to the field is the network used to communicate between devices of an IoT installation, a role that several wireless or wired technologies may fulfill:

### Addressability

The original idea of the Auto-ID Center is based on RFID-tags and unique identification through the Electronic Product Code, however, this has evolved into objects having an IP address or URI. An alternative view, from the world of the Semantic Web focuses instead on making all things (not just those electronic, smart, or RFID-enabled) addressable by the existing naming protocols, such as URI. The objects themselves do not converse, but they may now be referred to by other agents, such as powerful centralized servers acting for their human owners. Integration with the Internet implies that devices will use an IP address as a unique identifier. Due to the limited address space of IPv4 (which allows for 4.3 billion unique addresses), objects in the IoT will have to use the next generation of the Internet protocol (IPv6) to scale to the extremely large address space required. Internet-of-things devices additionally will benefit from the stateless address auto-configuration present in IPv6, as it reduces the configuration overhead on the hosts, and the IETF 6LoWPAN header compression. To a large extent, the future of the Internet of things will not be possible without the support of IPv6; and consequently, the global adoption of IPv6 in the coming years will be critical for the successful development of the IoT in the future.

### Short-Range Wireless

Bluetooth mesh networking—Specification providing a mesh networking variant to Bluetooth low energy (BLE) with increased number of nodes and standardized application layer (Models).
Light-Fidelity (Li-Fi)—Wireless communication technology similar to the Wi-Fi standard, but using visible light communication for increased bandwidth.
Near-field communication (NFC)—Communication protocols enabling two electronic devices to communicate within a 4 cm range.
QR codes and barcodes—Machine-readable optical tags that store information about the item to which they are attached.
Radio-frequency identification (RFID)—Technology using electromagnetic fields to read data stored in tags embedded in other items.
Thread—Network protocol based on the IEEE 802.15.4 standard, similar to ZigBee, providing IPv6 addressing.
Transport Layer Security—Network security protocol.
Wi-Fi—Widely used technology for local area networking based on the IEEE 802.11 standard, where devices may communicate through a shared access point.
Wi-Fi Direct—Variant of the Wi-Fi standard for peer-to-peer communication, eliminating the need for an access point.
Z-Wave—Communication protocol providing short-range, low-latency data transfer at rates and power consumption lower than Wi-Fi. Used primarily for home automation.
ZigBee—Communication protocols for personal area networking based on the IEEE 802.15.4 standard, providing low power consumption, low data rate, low cost, and high throughput.

### Medium-Range Wireless

HaLow—Variant of the Wi-Fi standard providing extended range for low-power communication at a lower data rate.
LTE-Advanced—High-speed communication specification for mobile networks. Provides enhancements to the LTE standard with extended coverage, higher throughput, and lower latency.
OpenWare—four-phase commit protocol discussed later that provides extended range up to 1 mile with an omni-directional antenna, or 30 miles with a directional antenna, all at lower power use than all other wireless protocols mentioned in this document including Bluetooth Smart. It also provides the best overall security model of any wireless protocol mentioned in this article.

### Long-Range Wireless

Low-power wide-area networking (LPWAN)—Wireless networks designed to allow long-range communication at a low data rate, reducing power and cost for transmission. Available LPWAN technologies and protocols: LoRaWan, Sigfox, NB-IoT, Weightless.
Very small aperture terminal (VSAT)—Satellite communication technology using small dish antennas for narrowband and broadband data.
Long-range Wi-Fi connectivity

### Wired

Ethernet—General purpose networking standard using twisted pair and fiber optic links in conjunction with hubs or switches.
Multimedia over Coax Alliance (MoCA)—Specification enabling whole-home distribution of high definition video and content over existing coaxial cabling.
Power-line communication (PLC)—Communication technology using electrical wiring to carry power and data. Specifications such as HomePlug utilize PLC for networking IoT devices.

### Simulation

IoT modeling and simulation (and emulation) is typically carried out at the design stage before deployment of the network. Network simulators like OPNET, NetSim and NS2 can be used to simulate IoT networks. Digital Twins may also be implemented to produce updates on the status and health of an asset, based upon sensor readings integrated with a computational model of the asset.

### Politics and Civic Engagement

Some scholars and activists argue that the IoT can be used to create new models of civic engagement if device networks can be open to user control and inter-operable platforms. Philip N. Howard, a professor and author, writes that political life in both democracies and authoritarian regimes will be shaped by the way the IoT will be used for civic engagement. For that to happen, he argues that any connected device should be able to divulge a list of the “ultimate beneficiaries” of its sensor data and that individual citizens should be able to add new organizations to the beneficiary list. In addition, he argues that civil society groups need to start developing their IoT strategy for making use of data and engaging with the public.

### Government Regulation on IoT

One of the key drivers of the IoT is data. The success of the idea of connecting devices to make them more efficient is dependent upon access to and storage & processing of data. For this purpose, companies working on IoT collect data from multiple sources and store it in their cloud network for further processing. This leaves the door wide open for privacy and security dangers and single point vulnerability of multiple systems. The other issues pertain to consumer choice and ownership of data and how it is used. Presently the regulators have shown more interest in protecting the first three issues identified above.

### Current Regulatory Environment:

A report published by the Federal Trade Commission (FTC) in January 2015 made the following three recommendations:
Data security—At the time of designing IoT companies should ensure that data collection, storage and processing would be secure at all times. Companies should adopt a “defence in depth” approach and encrypt data at each stage.
Data consent—users should have a choice as to what data they share with IoT companies and the users must be informed if their data gets exposed.
Data minimization—IoT companies should collect only the data they need and retain the collected information only for a limited time.

However, the FTC stopped at just making recommendations for now. According to an FTC analysis, the existing framework, consisting of the FTC Act, the Fair Credit Reporting Act, and the Children's Online Privacy Protection Act, along with developing consumer education and business guidance, participation in multi-stakeholder efforts and advocacy to other agencies at the federal, state and local level, is sufficient to protect consumer rights.

A resolution passed by the Senate in March 2015, is already being considered by the Congress. This resolution recognized the need for formulating a National Policy on IoT and the matter of privacy, security and spectrum. Furthermore, to provide an impetus to the IoT ecosystem, in March 2016, a bipartisan group of four Senators proposed a bill, The Developing Innovation and Growing the Internet of Things (DIGIT) Act, to direct the Federal Communications Commission to assess the need for more spectrum to connect IoT devices.

Several standards for the IoT industry are actually being established relating to automobiles because most concerns arising from use of connected cars apply to healthcare devices as well. In fact, the National Highway Traffic Safety Administration (NHTSA) is preparing cybersecurity guidelines and a database of best practices to make automotive computer systems more secure.

### Platform Fragmentation

IoT suffers from platform fragmentation and lack of technical standards a situation where the variety of IoT devices, in terms of both hardware variations and differences in the software running on them, makes the task of developing applications that work consistently between different inconsistent technology ecosystems hard. Customers may be hesitant to bet their IoT future on a proprietary software or hardware devices that uses proprietary protocols that may fade or become difficult to customize and interconnect.

IoT's amorphous computing nature is also a problem for security, since patches to bugs found in the core operating system often do not reach users of older and lower-price devices. One set of researchers say that the failure of vendors to support older devices with patches and updates leaves more than 87% of active devices vulnerable.

### Data Storage and Analytics

A challenge for producers of IoT applications is to clean, process and interpret the vast amount of data which is gathered by the sensors. There is a solution proposed for the analytics of the information referred to as Wireless Sensor Networks. These networks share data among sensor nodes that are send to a distributed system for the analytics of the sensory data.

Another challenge is the storage of this bulk data. Depending on the application there could be high data acquisition requirements which in turn lead to high storage requirements. Currently the internet is already responsible for 5% of the total energy generated and this consumption will increase significantly when we start utilizing applications with multiple embedded sensors.

### Security

Concerns have been raised that the Internet of things is being developed rapidly without appropriate consideration of the profound security challenges involved and the regulatory changes that might be necessary.

Most of the technical security issues are similar to those of conventional servers, workstations and smartphones, but the firewall, security update and anti-malware systems used for those are generally unsuitable for the much smaller, less capable, IoT devices.

According to the Business Insider Intelligence Survey conducted in the last quarter of 2014, 39% of the respondents said that security is the biggest concern in adopting Internet of things technology. In particular, as the Internet of things spreads widely, cyber attacks are likely to become an increasingly physical (rather than simply virtual) threat. In a January 2014 article in Forbes, cyber-security columnist Joseph Steinberg listed many Internet-connected appliances that can already “spy on people in their own homes” including televisions, kitchen appliances, cameras, and thermostats. Computer-controlled devices in automobiles such as brakes, engine, locks, hood and trunk releases, horn, heat, and dashboard have been shown to be vulnerable to attackers who have access to the on-board network. In some cases, vehicle computer systems are Internet-connected, allowing them to be exploited remotely. By 2008 security researchers had shown the ability to remotely control pacemakers without authority. Later hackers demonstrated remote control of insulin pumps and implantable cardioverter defibrillators. David Pogue wrote that some recently published reports about hackers remotely controlling certain functions of automobiles were not as serious as one might otherwise guess because of various mitigating circumstances; such as the bug that allowed the hack having been fixed before the report was published, or that the hack required security researchers having physical access to the car prior to the hack to prepare for it.

The U.S. National Intelligence Council in an unclassified report maintains that it would be hard to deny “access to networks of sensors and remotely-controlled objects by enemies of the United States, criminals, and mischief makers . . . . An open market for aggregated sensor data could serve the interests of commerce and security no less than it helps criminals and spies identify vulnerable targets. Thus, massively parallel sensor fusion may undermine social cohesion, if it proves to be fundamentally incompatible with Fourth-Amendment guarantees against unreasonable search.” In general, the intelligence community views the Internet of things as a rich source of data.

As a response to increasing concerns over security, the Internet of Things Security Foundation (IoTSF) was launched on 23 Sep. 2015. IoTSF has a mission to secure the Internet of things by promoting knowledge and best practice. Its founding board is made from technology providers and telecommunications companies including BT, Vodafone, Imagination Technologies and Pen Test Partners. In addition, large IT companies are continuously developing innovative solutions to ensure the security for IoT devices. As per the estimates from KBV Research, the overall IoT security market would grow at 27.9% rate during 2016-2022 as a result of growing infrastructural concerns and diversified usage of Internet of things.

In 2016, a distributed denial of service attack powered by Internet of things devices running the Mirai malware took down a DNS provider and major web sites.

Security experts view Internet of things as a threat to the traditional Internet. Some argue that market incentive to secure IoT devices is insufficient and increased governmental regulation is necessary to make the Internet of things secure.

The overall understanding of IoT is essential for basic user security. Keeping up with current anti virus software and patching updates will help mitigate cyber attacks.

### Design

Given widespread recognition of the evolving nature of the design and management of the Internet of things, sustainable and secure deployment of IoT solutions must design for “anarchic scalability.” Application of the concept of anarchic scalability can be extended to physical systems (i.e. controlled real-world objects), by virtue of those systems being designed to account for uncertain management futures. This “hard anarchic scalability” thus provides a pathway forward to fully realize the potential of Internet-of-things solutions by selectively constraining physical systems to allow for all management regimes without risking physical failure.

Brown University computer scientist Michael Littman has argued that successful execution of the Internet of things requires consideration of the interface's usability as well as the technology itself. These interfaces need to be not only more user-friendly but also better integrated: “If users need to learn different interfaces for their vacuums, their locks, their sprinklers, their lights, and their coffeemakers, it's tough to say that their lives have been made any easier.”

### Environmental Sustainability Impact

This section does not cite any sources. Please help improve this section by adding citations to reliable sources. Unsourced material may be challenged and removed. (November 2015) (Learn how and when to remove this template message) A concern regarding Internet-of-things technologies pertains to the environmental impacts of the manufacture, use, and eventual disposal of all these semiconductor-rich devices.

Modern electronics are replete with a wide variety of heavy metals and rare-earth metals, as well as highly toxic synthetic chemicals. This makes them extremely difficult to properly recycle. Electronic components are often incinerated or placed in regular landfills. Furthermore, the human and environmental cost of mining the rare-earth metals that are integral to modern electronic components continues to grow. With production of electronic equipment growing globally yet little of the metals (from end-of-life equipment) are being recovered for reuse, the environmental impacts can be expected to increase.

### Traditional Governance Structures

A study issued by Ericsson regarding the adoption of Internet of things among Danish companies identified a “clash between IoT and companies' traditional governance structures, as IoT still presents both uncertainties and a lack of historical precedence.” Among the respondents interviewed, 60 percent stated that they “do not believe they have the organizational capabilities, and three of four do not believe they have the processes needed, to capture the IoT opportunity.” This has led to a need to understand organizational culture in order to facilitate organizational design processes and to test new innovation management practices. A lack of digital leadership in the age of digital transformation has also stifled innovation and IoT adoption to a degree that many companies, in the face of uncertainty, “were waiting for the market dynamics to play out”, or further action in regards to IoT “was pending competitor moves, customer pull, or regulatory requirements.” Some of these companies risk being ‘kodaked’—“Kodak was a market leader until digital disruption eclipsed film photography with digital photos”—failing to “see the disruptive forces affecting their industry” and “to truly embrace the new business models the disruptive change opens up.” Scott Anthony has written in Harvard Business Review that Kodak “created a digital camera, invested in the technology, and even understood that photos would be shared online” but ultimately failed to realize that “online photo sharing was the new business, not just a way to expand the printing business.”

### IoT System and Hardware/Software/Networking Design and Implementation

A typical IoT solution integrates multiple technologies to solve a specific business problem using the following key components:
Edge: Embedded technology that senses, acquires and sends data.
IoT Platform: Accepts, ingests, stores and analyzes IoT data. Includes several features common to an IoT implementation including machine-learning, artificial intelligence (AI), business analytics, integration services for connecting to the existing Enterprise computer software tier, an Edge device registry, as well as messaging middleware.
Enterprise: Applications, processes or services that act upon intelligence from the data.

IoT is ultimately about the data and how it can be used to help companies improve operational efficiencies, drive new revenue streams and provide customer insights. The IoT Platform currently includes such feature sets as machine-learning, artificial intelligence (AI), business analytics, integration services for connecting to the existing Enterprise computer software tier, as well as middleware servers for connecting to the Edge devices. The Edge tier described in this disclosure will be able to encapsulate several of the feature sets contained in the IoT Platform such as AI, machine learning, and Edge devices can also be reprogrammed so that over time they can be “taught” by software and configurations created by the IoT Platform tier. This ongoing “learning cycle” may take several forms depending on where the data resides to figure out how to improve the overall system and which Edge devices need to be reconfigured or reprogrammed to carry out the “learned” behavior the system learns over time through the use of machine learning and AI techniques.

### IoT's Impact on Business

Businesses that lag behind the technology curve may never be able to bridge the gap. As a general example, Airborne Express used to be one of the top three overnight shipping companies, competing directly with UPS and FedEx. However, they did not see technology as a strategic enabler and failed to keep pace with their key competitors. Eventually, the chasm was so great that they were no longer able to compete on service or price and had to sell the company.

IoT has the potential of being the most disruptive and transformative technology to affect both IT and business in years. The IoT revolution creates unprecedented opportunities for businesses to provide enhanced customer experiences, build new customer communities, create a new generation of products, provide advertising that is totally relevant to the targeted audience, improve operations and reduce costs. Today's customers of digital devices expect smarter, connected and technologically advanced systems, and the companies that sell them expect to have data analytics that enable them to achieve operational efficiencies.

### How Businesses are Harnessing IoT

Here are a few examples of how companies are gaining a competitive edge and changing their businesses through IoT.

The American cruise ship company Royal Caribbean used IoT to reduce costs, increase revenue and improve workflows. By integrating sensors and their onboard point-of-sale systems, tablets, signage, TV, photo gallery and ticketing systems—then harnessing the resulting “ocean” of data—they now have a better understanding of their guests' needs and can tailor and personalize guest experiences. They have also been able to streamline the food temperature inspection process and cut the temperature check times by 60%. Royal Caribbean now has an intelligent system that captures and makes sense of data flowing across systems at every level of the ship.

IoT is also affecting our global food supply. Farmers today are under significant pressure to do more with less, all while managing greater operational complexity. As a result, John Deere began connecting its farm equipment to a mobile platform, giving farmers and their dealers remote access to fleet location and utilization as well as diagnostic data for each machine. They are also using networked sensors combined with historical and real-time data on weather, soil conditions and crop status to ensure the right crops are planted at the right time and place.

### Why Companies Need IoT

IoT provides a tremendous opportunity across all segments of a corporate entity. There are many potential scenarios that could be considered, including:
Creating new customer experiences and customer communities (Customer Service, Consumer Products)
Driving operational efficiencies, predictive maintenance and more intelligent supply chains across the organization
Enhancing the fan/viewer experience for athletic events using biometric sensors on participating athletes (Media Networks)
Driving automation/efficiencies in manufacturing facilities

Power management and the lack of efficient mid-range wireless capabilities (over 300 feet) are two of the biggest challenges facing IoT implementations. TerraTrace Sensor Integration Packs provide over 400 hours of use of multiple sensors with a standard coin-cell battery which can easily be replaced when needed, or powered by a rechargeable battery that can run over 250 hours continuously per charge. In addition, the TerraTrace SIP wireless protocol can transmit up to half a mile line-of-sight, and can be extended if needed to one mile. Data is encrypted from the SIPs all the way to Azure using TerraTrace's proprietary wireless protocol and SSL encrypted pipes as needed. Our wireless protocol has a 4-phase commit, which guarantees packet delivery and goes well beyond the stability of platforms built on Wi-Fi, Bluetooth or Zigbee, making it ideal for mission critical applications such as healthcare. In addition to enhanced mid-range wireless capabilities, TerraTrace also provides long-range backhauling using any of the 550 GSM cellular networks available globally or the Internet.

Current sensor capabilities include:

Magnetic integration

Different transmission capabilities

Sleep modes

Temperature down to 1/10th degree F. in accuracy

G-Forces down to 1/10th degree accuracy (digital)

RFID (integrated for a customer 7 years ago)

For large amounts of collected data, SD cards can be used to store the data to be batch uploaded to avoid data fees or the data can be streamed in real-time. Recommended to tether and batch to minimize battery drainage.

All of these capabilities are literally sitting on the shelf waiting for a customer to use them.

### Sensor Types

General Monitoring
Temperature
Motion
Humidity
Door & window status
Light
Dust
Pressure
Vibration
Mechanical shock
Combustible Gases
Toxic or organic gases
Indoor pollutants
Automotive ventilation
Cooking vapors
Oxygen
Electrical flow
Amount of water used

IoT Edge Short Range Wireless Sensor Pack Options (EDGE-Sensor type): Capable of monitoring ID and sensor readings with battery condition, reporting any changes at a preprogrammed time interval. These sensor packs are able to “send” data (transmitter) to the IoT Edge Router or Hand-held for forwarding to either the Local host, Intranet or Internet database via Azure. All models are available with a non-rechargeable coin cell battery offering 400 hours of continuous use, or with a rechargeable battery offering 250 hours of continuous use in the same form factor. All have a standard transmission range of 2000 feet line on wireless range with a four-phase commit per transmission to guarantee delivery.

IoT Edge Sensor Pack—Client can specify sensor type from tested sensor type list (1 k minimum order)
Example: Temperature (EDGE-T), Electrical flow

### Operating Specifications

Reporting frequency: 5+ minute intervals
Can be preset at factory per clients request-1 k minimum order to change intervals
Dimensions: ¾ in×¾ in×1.5 in length w/rounded corners and mounting tabs
(19 mm×19 mm×38 mm)
Optional-no mounting tabs
Magnet: Optional—Neodymium magnet with 7 lbs of holding force.
Battery Life: 5 years minimum
Unique wakeup condition (deep sleep until sensor is ready to be deployed)
Transmission distance: 240 feet (72 m) in most liquids, 2000 feet (608 m) in open air
Reports: Unique tamper-proof identification, temperature (core temperature with changes of 0.1+/−degrees F.), and battery condition
One visual display indicators (green LED)
Locating low power RF signal (ID only) every 10 Seconds up ta distance of 10-15 Feet
RF frequency range 433 MHz or 915 MHz

IoT Edge Smart Sensor Pack (EDGES-Sensor type): Capable of sending permanent ID and, monitoring Battery condition, three axis motion with up t200 “G force” impact range and one client specified sensor condition. This sensor pack is able to “exchange” data (transceiver) from the Router or Hand-held for forwarding to either the Local host, Intranet or Internet database via Azure.

### Operating Specification:

Reporting default frequency: 5+ minute intervals
Can be preset at factory per clients request-1 k minimum order to change intervals
Interval can be modified in the field or from remote location
Two way commutation from the field (local Router and/or Hand held) or from remote location (Local host, Intranet/Internet)
Immediate alarm if measurement exceeds predefined window
Alarm measurement can be modified in the field or from remote location
Client option to request acknowledged data received message from sensor
Dimensions: ¾ in×¾ in×1.5 in length w/rounded corners and mounting tabs
Standard Size: (19 mm×19 mm×38 mm)
Optional-no mounting tabs
Various custom sizes can be achieved
Magnet: Optional—Neodymium magnet with 7 lbs of holding force
Battery Life: 5 years minimum
Two visual display indicators (green LED—on/send, Blue—receive data)
Unique wakeup condition (deep sleep until sensor is ready to be deployed)
Transmission Distance: 240 feet (72 m) in most liquids, 2000 feet (608 m) in open air

Reports: Unique tamper-proof identification, Battery condition, sensor data, 3-axis motion sensor with “G” force info, Signal strength and a locating low power RF signal (ID only)

For locating within the area: The EDGES Sensor is capable of receiving a “locate” command then start sending a reducing power burst every 10 seconds until a 10 ft (3 m) radius exists. The locating packets can be received by either the hand held Mini-Router or the nearest Router

RF Frequency Range 300 MHz t960 MHz

IoT Edge Smart Sensor Pack w/SD Card (EDGESD-Sensor type): Capable of sending permanent ID and, monitoring Battery condition, three axis motion with up t200 “G force” impact range, two internal client specified sensor conditions and with the option of adding a plug-in external sensor probe. An internal “SD” memory card of up t16 GB allows retention of both the created sensor data and also the information sent from the Router to store. This sensor pack is able to “exchange” data (transceiver) from the EDGE Router or Hand-held for forwarding to either the Local host, Intranet or Internet database via Azure.

### Operating Specification:

Reporting default frequency: 5+ minute intervals
Can be preset at factory per clients request-1 k minimum order to change intervals
Interval can be modified in the field or from remote location
Two way commutation from the field (local Router and/or Hand held) or from remote location (Local host, Intranet/Internet)
Immediate alarm if measurement exceeds predefined window
Alarm measurement can be modified in the field or from remote location
Sensor data compared to previous reading, if no change, records then allows client the option to forward matching data
Allows client to control transmission data timing
Client option to request acknowledged data received message from sensor
Create tamper-proof permanent records contained in the sensor
Dimensions: ¾ in×¾ in×2.5 in length w/rounded corners and mounting tabs
(19 mm×19 mm×52 mm)
Optional-no mounting tabs
Magnet: Optional-Neodymium magnet with 11 lbs of holding force.
Battery Life: 5 years minimum
Internal Memory Storage (2 GB to max of microSD card)
Full FAT32 file system (Windows compatible)
Data logging storage for full 5 years
Real time access tall data
Transmission Distance: 240 feet (72 m) in most liquids, 2000 feet (608 m) in open air

Reports: Unique tamper-proof identification, Battery condition, data from two custom embedded sensors and optional plug-in external sensor probe (client specified), 3-axis motion sensor with “G” force info, Signal strength, and any other data stored within the device and a locating low power RF signal (ID only).

For locating within the area: The EDGES Sensor is capable of receiving a “locate” command then start sending a reducing power burst every 10 seconds until a 10 ft (3 m) radius exists. The locating packets can be received by either the hand held Mini-Router or the nearest Router.
Optional infrared transmit capability
Dual visual display indicators (Red & Green LED)
Unique wakeup condition (deep sleep until sensor is ready to be deployed)
External voltage and current measurement capability
Optional interface with external sensors (humidity, magnetic, pressure, etc.)
RF frequency range 300 MHz t960 MHz
Sensor Capabilities: The following sensor types are within tested parameters of the EDGESD
Temperature (internal-PCB & external probe), *3-axis motion/vibration, * Mechanical shock (“G” force)
Humidity (internal-PCB & external probe), Pressure (barometric, 0 t125 psi—PCB & external probe)
Switched event (external probe: open/close, proximity/hall effect, toxic & combustible gas, solvents, etc.)
Additional sensor types can be added to either PCB or external probe per Clients requirements

IoT Edge Sensor Router/Coordinator (EDGER-Router, EDGEC-Coordinator): EDGE Router collects all data from its surrounding area, compares existing data from the specific sensor, then if no change, creates a data log file. If there are exceptions the system will then forward at the next scheduled reporting cycle to the Coordinator which is linked to the customer-preferred method of final data acquisition. This Router allows direct sensor contact (OTA) with any of our IoT Edge Sensor Packs from anywhere in the world.
Routers: The number needed is based on the logistics of the area to be monitored
Coordinator: One per client based Computer/Web interface

### Operating Specifications:

Power: Client specified—
AC Model—Transformer to AC source with 6 volts DC output or
Battery Operated Model—Stand alone or with wind and/or solar charging system
Coordinator only (EDGEC)—the above two options plus can be powered by USB port
Dimensions: 4.8 in×4.8 in×1⅞ in height (12.2 cm×12.2 cm×4.8 cm)
Reception coverage: 120-foot (36 m) radius for liquid sensors and 1000 foot (304 m) for open air
Antenna: Internal, Dipole w/2 foot (0.6 m) long cable, Dipole WIP, or Yagi (directional long range)
Router placement: Recommend 10 t14 feet high (3 m-4.3 m)
Reports: Customer specified with alerts/changes to requested data modified over the air (OTA)
Each Sensor input is time stamped
Each Sensor input creates a receive signal strength (RSSI)
Internal Temperature sensor
3 Axis motion with “G” force
2 GB SD card memory (upgradeable to max of microSD card)

Multiple Status conditions: Storage (Data logger), Release (Forwards all data in buffer), Pass through (Sends sensor data as received in real time), Compare (Allows same sensor data packets to create a running log w/timestamps of same value), Alert (sends only sensor data outside of preset Hi/Low values) and Change (reset command functions i.e. Hi/Low settings, reset Internal Clock, Report values, etc.)

### System Data Links:

Local host (Wi-Fi, CAT5, Zigbee, etc.)
Intranet link directly to clients existing system
Internet (GPS, GSM, GPRS and/or Satellite)
Any combination client deems necessary.
RF frequency range 300 MHz t2.4 GHz

IoT Edge Hand-Held Mini-Router (EDGEM/R): A mobile device to receive and/or sent data to any IoT Edge Smart Sensor Pack (receive only w/IoT Edge Sensor). Uses locating signal of sensors to pinpoint location and/or read/write to onboard memory. The EDGEM/R interfaces with existing Pads, Tablets, and Android phones using specially designed interface.

### Operating Specifications:

Power:

USB port—powered by standard link

Battery backup for saving data between link up with display device

Dimensions: 2 in×2 in×¾ in height (5 cm×5 cm×2 cm). Reception coverage: 120-foot (36 m) radius for liquid sensors and 1000 foot (304 m) for open air. Antenna: Internal and/or Dipole WIP (directional). Status LEDs—Power on, Receive data, Send data. Reports: Customer specified, over the air (OTA), requested data from/to sensor. 2 GB SD card memory (upgradeable to max of microSD card)

System Data Links: Local host (USB port) and/or Edge Router/Coordinator. Although Archetype works with global sensor manufacturers that provide over 140,000 sensor types, the following capabilities have already been integrated, tested, and are in production:

### General Monitoring:

Temperature
Motion
Humidity
Door/Window Status
Light
Dust
Smoke
Pressure (barometric, 0-150 psi)
Vibration
Mechanical shock
Electrical flow
Water amount used

### Combustible Gases

LP-Gas/Propane (500-10000 ppm)
Natural gas/Methane (500-10000 ppm)
General combustible gas (500-10000 ppm)
Hydrogen (50-1000 ppm)

### Toxic Gases

Carbon monoxide (50-1000 ppm)
Ammonia (30-300 ppm)
Hydrogen sulfide (5-100 ppm)

### Organic Solvents

Alcohol, toluene, xylene (50-5000 ppm)
Other volatile organic vapors (special order)
CFCs (HCFCs and HFCs)
R-22, R-113 (100-3000 ppm)
R-21, R-22 (100-3000 ppm)
R-134A, R-22 (100-3000 ppm)
Freon (100-3000 ppm)

### Indoor Pollutants

Carbon dioxide
Air contaminants (<10 ppm)

### Automotive Ventilation

Gasoline exhaust
Gasoline and diesel exhaust

### Cooking Vapors

Volatile vapors from food (alcohol)
Water vapors from food

### Oxygen

0-100%—5 year life, 12 sec t90% response
0-100%—10 year life, 60 sec t90% response

The above described Sensor Device, Router and Gateway designs could be used in and configuration of features and/or sensors in conjunction with any of the Sensor and Sensor Device, Router, and Gateway hardware/software/firmware combination designs mentioned herein.

The sensor packs mentioned in all previous filings, as well as this one, could be implemented as System on a Chip (SOC) designs to micronize the sensor pack setups. SOC designs are where multiple chips are combined into a single chip die. All sensor designs that are made as SOC designs could includes a processor, memory, and or RF transmitter, and one or more sensors on a single chip to dramatically improve power management and reduce the size of the sensor packs, as well as increases in overall efficiency. The SOC designs can also be applied to any of the sensor packs mentioned in the previously referenced patent designs. SOC designed sensor packs can be placed all over the head and/or body for monitoring all aspects of biological functions. These SOC designs, if considered as part of a network all over the head and body of a human or animal, can be wired to transmit sensor data to a central communication pack located somewhere else on the human or other animal, or can transmit information directly to an external computing device such as a tablet, phone, laptop as referenced in the previous patent filings. The conditions that can be monitored by the apparel include muscle fatigue, blood oxygenation, heart rate, lung rate, blood pressure, body heat, and any other neurological or cardiovascular condition mentioned in previous filings.

This patent discloses a novel concept for implementing a security scheme in a sensor based network. The system should include a sensor, a network (wired or wireless), redundant storage facilities along the transmission route, an endpoint storage facility (cloud based, Internet, or closed network), and a client interface that may be installed on a laptop/desktop or hosted for Internet access through a web interface. Currently, sensor network, implementations don't involve encryption from the sensors to the gateway devices, and rarely do the gateway devices involve any security either storing data locally in clear text or transmitting it to the storage facility in an insecure capacity. To be considered fully secured and non-tamperable, the data has to be encrypted all the way through the system in a manner that cannot be further altered from the source sensor. Normally the data, even if encrypted along one segment of the transmission path, will be decrypted and exposed in memory in clear text in several phases of the data transmission. However, several mechanisms can be employed to improve or maintain a high degree of security in a sensor based network.

One such mechanism would be to have the sensor be implemented with a processor and memory so that firmware can read the sensor directly as if on the same circuit board as the processor and memory or even in the same System-on-a-Chip (SOC) design. If the firmware can read the sensor data, then it can immediately apply logic and only transmit the data through the network in a fully encrypted manner. This will allow the system to be more efficient on transmission, whether on a wired or wireless network segment, as only relevant sensor data will be transmitted throughout the system. In such a mechanism, each transmission device should implement its' own storage and transmission logic so that data is written to memory in a round-robin approach as to not overwrite an existing data packet with new data until all the rest of the data storage has already been written over. If firmware from a gateway device receives a new data packet, it should already know the length of a valid packet and only write the packet to memory or internal storage if it is of valid size and the contents have been verified by a two or four-phase commit wireless or wired transmission scheme from the sensor pack itself. If the firmware works in the following capacity then it should maintain a “cursor” position or remember the end memory segment or storage location on disk and write the new packet in an unaltered state after the last segment recorded. If this logic is the only way to receive and transmit data through the network, then there is a dramatic reduction in the data being altered, modified, or otherwise tampered with. Such gateway devices could also support one-way data flow so there is the notion of a wireless or wired receiver, storage or live memory, and a wireless or wired transmitter per gateway. If this hardware/firmware/software design is maintained this will ensure data flows through the sensor network in a secure and unaltered state. This mechanism will also allow the most redundant data storage possible along the transmission path so that if the transaction fails at any one point, the same scheme can be used to maximize the possibility of data recovery along the transmission path.

Another such mechanism to be used in concert with or separately would be to manage a transaction through multiple pieces of transmission hardware along the transmission path so that the sensor pack starts an encrypted transmission which goes wired or wirelessly to another device, possibly a gateway device, and then a session is maintained on the gateway device while the gateway device transmits the encrypted packet of data to a server environment on the network for storage. Once the data is stored, then the server environment sends a successful response to the gateway device in a synchronous or asynchronous manner, and then the session on the gateway device can then end the transaction and session successfully. If the session isn't responded to in a timely fashion by the server or an unsuccessful transmission is registered back the gateway device, then the gateway device can invalidate the session and attempt to resend as a new transmission. If in turn the server responds at the same time the gateway device is retrying the packet transmission, then the gateway should ignore the response and continue to send the packet again. The server should in turn use an id in the packets coming in or timestamp to verify if the data packet has already been stored. If this logic is maintained in a thread-safe manner at the software level, the system can guarantee packet delivery without server-side storage duplication in a fully secured manner.

Both mechanisms can be used together or separately, but should both maintain a fully secured packet along the entire transmission path as well as maintain a round-robin in memory and on disk storage pattern within each device along the transmission path. This will dramatically reduce the chances of the data being modified or corrupted in any way along the transmission path while ensuring maximum redundancy and recoverability throughout the sensor based network. The initial sensor or sensor packs can consist of one or more of a single type or multiple types of sensors in use. The same system may have multiple hardware devices between the sensor pack and the primary storage facility, and the round-robin memory storage mechanism as well as the transaction management through devices could be employed throughout the entire transmission path. Data should also be stored in a centralized storage facility that will follow the same scheme in an encrypted or unencrypted manner to ensure data is not corrupted or modified from the sensors in use in any way. This will ensure data integrity throughout the system and also render the data court admissible for any purpose.

For client access along the transmission path or from the Internet, the data can then be read into memory, decrypted, and logic applied to offer a summary or quantified view of the data for use in a variety of applications. Any of these transmission schemes may involve laptops, tablets, smartphones, desktops, or server appliances, or any other computing device that can receive and sent data through a sensor based network.

This patent discloses several novel concepts related to the sensor implementations described in previous filings, as well as additional security schemes that could be incorporated into sensor, Internet enabled, smart phone, and/or mobile device based networks, as well as how to best apply sensor implementations to navigate and manage robotic-based systems.

To further understand the nature of these inventions, it is important to first understand the four-phase commit transaction protocol that was described in previous filings. The four phase commit transaction model involves several phases to guarantee message delivery between the sensor and the receiver or server environment. The first phase is where the sensor or sensor integration pack (consisting of a sensor, processor, memory, and/or standalone power source, and two-way transmission radio) sends a packet of data to the receiver (hardware consisting of a radio, processor, memory and/or standalone power source). The data may or may not be encrypted during transport. The packet may consist of a header and/or a body of information, as well as a unique id which may be specific to the packet sent as well as the sensor pack id of the transmitter sending the packet. Once the packet is received, the receiver will read the packet and prepare a response. This packet is then analyzed to measure the length and/or the contents of the packet transmitted. A checksum may then be generated that represents the amount and/or contents of the data received. The checksum along with potentially other identifiable information of the initial transmission is then sent back to the sensor pack as the second phase of the four-phase commit. Then the sensor pack reads the response, parses out the information which may include a unique id and/or the checksum data to verify the amount and/or contents of the data transmission. Then, the sensor pack may compare the checksum to the data initially sent as part of the transaction. The sensor pack may compare any data points send by the receiver to determine whether or not the packet was read in its' entirety, or whether additional information needs to be sent as part of the same transaction to continue delivering data associated with the initial packet. The latter portion of the decision-making process by the sensor pack may be to determine if the transaction requires multiple packets to be sent to successfully complete the transaction for the entire four phase commit process, or whether or not the initial transmission was successful and should be completed. If additional data needs to be sent as part of the same transaction, then additional information will be sent from the sensor pack to the receiver as part of the process in the same manner as described in the first phase of the four phase transmission sequence. At this point in the four-phase commit transaction, if the sensor pack determines that it has sent the last packet of data, or if the initial packet of data was the entire payload for the transmission, then the transmission for the entire transaction will be analyzed for success of failure. If deemed successful, then the sensor pack will send a final confirmation that the transaction was successfully executed as part of the four phase commit protocol and will clear the transaction from its' sending queue. This may also require that the sensor pack store the transaction in memory locally for retrieval later if needed, possibly in the round robin mechanism that was described in previous filings. If any aspect of the response from the receiver of the transmission is deemed a failure by the sensor pack and/or receiver, then the sensor pack will send a failure response back to the receiver as the final step in the four-phase commit to have the receiver reverse out the commit of such data to storage and allow the entire process to restart from the sensor pack. If failure is detected by the receiver, it should send a failure response back to the sensor pack and clear the transaction so the sensor pack can begin the transaction again when appropriate. This will ensure complete data integrity across networks where a single sensor pack is communicating to a single receiver, as well as in cases where there are several sensor packs communicating with several receivers locally all the way up to one or more server environments where data is finally stored for the transaction. In an environment where several receivers are in range of a sensor pack, then the sensor pack should only accept one receiver response, and only communicate with that receiver until the transaction is completed. This mechanism applies if only one data payload is involved as well as if multiple data packet transmissions are needed to complete the transaction. The server environment may be part of a distributed network or a centralized data storage facility. All phases of the communication can be encrypted on a transaction level using the same encryption scheme, or variants of encryption can be used during the process per transmission to further increase security. One-way modulation of the encryption can be applied is to vary the encryption scheme based on information collected during the four-phase commit process. In other words, the checksum values could be used to choose another encryption scheme, or the id of the sensor pack or originator of the packet transmission can be used to further randomize the encryption formula or seed data for hash algorithms during processing of subsequent phases of the transmission. The preferred mechanism for encryption is AES 128 or AES 256 encryption. Another aspect of this protocol may be that it doesn't have to initiate a handshake transmission to start the transaction. By eliminating this handshake phase on each transmission, the radio protocol becomes more efficient on power management as well as increases performance over other radio protocols that require a handshake to initiate data transmission.

The same four-phase commit transmission protocol described above could be used in an Internet Protocol enabled network, or other closed computing network where one or more computing devices may communicate with one or more computing devices and data has to be delivered in a guaranteed way that cannot be tampered with. To better understand the uniqueness of this protocol, one must understand that Internet Protocol is currently a two-phase commit process. In other words, one computing device such as a server sends data to another computing device such as a client (in server/client networks), and the second computing device simply sends back a basic response, sometimes referred to as an “ACK”, to let the initial computer know that it can send more data. This transaction model is only two phases and does nothing to secure data or guarantee transmission of each individual packet exactly down to the bit level. The four-phase commit protocol does precisely that and ensures that every packet on the network was not only sent in its' entirety, but can verify the sender's identity to a much more stringent level, thereby making it an ideal model for data security on a computer network.

The next disclosure is to enhance the security aspects of a four-phase commit model, or possibly a two-phase commit model using similar security techniques. If during either transaction model, the receiver determines that the sender is not an authorized sender, or the data has been deemed to be tampered with or injected from an unauthorized source, then the receiver (server or other computing device capable of processing data) can simply block traffic from the sender. However, other more active approaches can be used to stop unwanted data from being accepted by the receiver. One such mechanism could be an inverse of the Ransomware attack model in computer networks, whereby the receiver can initiate an attack back on the sender computing device. To better explain, one must know what a Ransomware attack is. Ransomware is where software is installed on a host computing device that will when triggered will encrypt some or all of the contents of the host machine and hold the data “ransom” while anyone trying to access the data will have to pay someone or entity money or take other actions before the person or system performing the Ransomware attack will provide a key that will decrypt the data being held ransom. This could also take the form of providing some other information that will allow the user to access the encrypted data upon compliance. If a similar approach is deployed in this security model, the receiver of the network request or transmission could then send a request or software program back to the sender to have the computing device that initiated the transmission encrypted in part or in full so that the receiver has ended the attack from the sending computing device. This mechanism could then allow the sender to verify that they weren't attempting to compromise the receiving computer device or network and in turn be provided information or have a request sent out from the receiver device or network to allow the sender access to their computing device immediately or at some point in the future. This in effect could allow a receiving computer device or network to stop unwanted requests to it in a proactive manner that wouldn't permanently disable or destroy the sending computing device. This could also happen at the protocol level so that upon initial transmission from the sender, the receiver could verify if the packet is valid and from a valid source and then decide to receive the packet for further processing, or if the packet is invalid and/or from an invalid source initiate the reverse Ramsomware-like protection attack on the sending computer device to stop it from continuing to send invalid data to the receiver computing device or network. The security model could take other forms of active denial against the sending computing device or network to stop any unwanted transmissions from being received.

This patent discloses several novel concepts related to computer security implementations on computer security measures described in previous filings and related to the “Internet of Things” computer architecture. For securing computer networks, one must consider better login schemes. Current login schemes for computer based and Internet enabled systems just require a unique username and password combination to access the system. Such login schemes can be used on any computer or device as long as the username and password is accurate. The new login scheme proposed for a more secure network or computer access would require not only a unique username, but could also use a password that uses a user input password such as from a keyboard or from mouse or screen clicks and combines it via a secure hash algorithm or other joining algorithm that combines the password with the MAC address, IP address, or other information referenced uniquely on the device itself. The password could also be joined to any other information that is specific to the computing device the user owns or could be additional biometric information such as a fingerprint, facial recognition, retinal scan, speech analysis, or other biographical information to generate a number sequence that can then be joined to the password. Such methods for joining the user supplied password with the additional biological or computing device-specific information could be through hash algorithms, XOR, or any ad hoc algorithm that would further obscure the initial password from the user. This same scheme could also not involve user supplied passwords but simply use an initial setup sequence that would involve the user registering their speech pattern, face pattern, fingerprint, retinal scan or other biological information unique to the individual that may or may not be registered under a username which may or may not be an email address to login to the account. Then new signups could compare this information to existing profiles of users to make sure the user has a single account that cannot be compromised. This will ensure identity of the user to other users of the system and vice versa. This scheme would also reduce the likelihood of social hacking for passwords as logins would require the user as well as their specific hardware to be used to login to the system or secure social media network. This will ensure no accounts are logged into through primary current hacking techniques. The username could alternately be just an internal or assigned username that is given or allowed to be changed by the user upon initial unique account signup. This will make the system easier to use and more difficult to hack through current hacking techniques.

The Internet of Things is a new style of architecture that will connect every product electronically, and most likely wirelessly, to the Internet. Many device manufacturers are currently building in sensors with radio communication that would allow the product's internal status, usage patterns, or other information regarding operation or process to be sent out via radio signal to hardware devices that can listen to their communication and transmit that communication to the Internet, or have a computer hardware or software system on the Internet that could send information to the product and have it respond in kind. This two-way communication between the product and the Internet is now being referred to as the “Internet of Things” computing architecture. The means by which the products will primarily communicate to the Internet will be through hardware devices known as gateways and/or routers that can send and/or receive the signals from the product. These communications may or may not occur over a cable and/or “short range” and/or “mid range” communications such as WiFi, RFID, Zigbee, Bluetooth, Openware (our own four-phase commit protocol described in detail in previously mentioned filings), LoRaWAN (LoRa), Sigfox, cellular, satellite, or any other ad-hoc wireless communications protocol in any combination, and then send them to the Internet via a dedicated or intermittent Internet connection (which may be in turn wireline or wireless, or any other combination mentioned above). The routers or gateway devices that are currently available are devices such as Rasberry PI, Android devices, etc. Although these devices will work in limited capacity, they are in no way equipped to handle multiple short range transmission protocols “out of the box” and are not capable of connecting all products in a local environment to a single gateway or router device. One new router/gateway device design could be a hardware design that can scan a household, manufacturing facility, or other local region for wireless transmissions such as radio signals. Then decode the signal into raw data that the gateway/router device can understand. These wireless transmissions can be WiFi, RFID, Zigbee, Bluetooth, Openware, LoRaWAN (LoRa), Sigfox, cellular, satellite, or any other form of “short range”, “mid range”, or “long range” radio signal protocol or airborne signal otherwise that may be used for IoT systems. Once the signal is decoded, the router can then scan for such signals on a scheduled interval or permanently as to act as a receiver for the signal detected. This will in turn allow the gateway/router to undergo an initial setup routine to decode all signals coming from any radio frequency enabled devices or products and then normalize them into a language the gateway/router can understand. The gateway/router can then transmit the normalized information from one or more devices or products to the Internet such as a cloud environment like Microsoft's Azure platform, Amazon's AWS (Web Services) platform, or some other computer network residing on the Internet or computer communications network. The data can then possibly be stored and/or used to drive business processes such as rules engines or business workflows in real time or at some point in the future. Such processes could include emailing parties when certain information indicates they be notified. As an example, if a refrigerator warms to a certain level that would indicate the cooling system is failing, then a service technician can be notified via text message, email, or other form of communication. The service technician can then be instructed to come out for a service check and possibly fix the refrigerator before all the food spoils. The gateway/router can also support devices being connected by cable directly as opposed to wirelessly for communications.

The scanning mechanism described above can be designed in the following ways. The gateway/router can first scan for a specified period to see which products are transmitting information and record which frequencies, baud rates, and/or additional product information can be picked up through real time detection and/or decryption and/or decoding of individual packets of wireless transmission data. All aspects of the different types of communication received from the product(s) in the local environment by the gateway/router should be recorded. The protocol format(s) that are detected can then be looked up via a database on the gateway/router and the product type(s) and wireless transmission type(s) can be recorded as a local wireless profile for the gateway/router to immediately and/or in the future. The information collected by the scan may also be sent to the Internet for decryption or decoding of the transmission type via a product wireless protocol catalog kept in a database on the Internet. The product type(s) and wireless transmission type(s) can then be send back to the gateway/router as a profile so the gateway/router knows how to communicate with each product in the local environment. This information can be stored and/or used for immediate and/or future use. Once the local “short range”, “mid range”, and/or “long range” network protocol(s) are deciphered and/or decoded and the gateway/router knows how to send and receive data transmissions to and from the product(s), then the gateway/router can then poll the different frequencies and baud rates to receive any transmissions from the products on a scheduled or one-time interval. The gateway/router may implement one or more antennas to perform the sending and receiving of transmissions to different products, if more than one product is sending and/or receiving transmissions. If a single antenna is used to communicate with multiple products, then the gateway/device will have to reprogram the antenna and/or computer logic on the gateway/device driving the antenna reception on a programmed time interval or for a single invocation to be able to send and receive on different wireless protocols on scheduled intervals. In other words, the antenna will have to be tunable to receive different frequencies and/or baud rates from potentially different pick parts and possibly additional information if needed to perform having a single antenna send and receive communications from multiple products. One example of this type of single antenna/multiple wireless protocol in use implementation is if there are five products that can transmit sensor information to the gateway/router. The gateway/router will need to cycle through the different protocols/product types profile created in the setup to scan for all product communications in a given interval at a rate that collectively doesn't exceed the maximum amount of time the products will try to resend information. In other words, if all five products will attempt to transmit for 1 minute before cancelling their transmission to the gateway/router, then the gateway/router will scan on each frequency and baud rate for no more than 12 seconds at a time in a single cycle so that the gateway/router can detect any transmission from any product before the product decides to cancel the transmission. Since there are 5 products in the local environment, 12 seconds of scan for communications from each product will result in 1 minute cycles for scanning all products. This will ensure that one gateway/router device always receives communication initiated by a product. If the gateway/router is designed with multiple antennas, each antenna could be utilized in a way to talk to multiple products or a single individual product per antenna. If each product has a dedicated antenna, then the cycling of scanning for an individual product can be eliminated as each antenna can be constantly listening for communications from each individual product. Additional information such as pick part type used by the manufacturer and any encryption-scheme specific information or other information may be needed to determine how to decrypt and/or decode the data from the products or transmit information to the products, both of which should be enabled by such a system. Specific product wireless profiles could be built into the gateway/router by the manufacturer and/or configured in advance of deployment, or pushed to the gateway/router so that the scanning mechanism is not needed and the gateway/router is shipped to the customer already configured to communicate with certain products and/or product types, or the wireless profile configuration can be controlled by an interface on the Internet via a cloud-based web interface or any other computer interface such as a mobile device, tablet, etc.

The gateway/router design should implement several security features that will ensure no firmware or data transmissions are ever tampered with or intercepted in clear text. This will require the data transmissions be encrypted from the sensor pack all the way through the gateway/router to the Internet as well as to the client interface. The firmware should be signed through a code certificate mechanism and written to read only data storage on all sensor packs as well as gateway/router devices to ensure no tampering with the hardware. A unique id should be assigned to each piece of hardware used in the system in advance of deployment so that each piece of equipment can be uniquely identified in the system. Data that is no longer needed should be erased from local memory so that no device can retain information sent to or received by the sensors. There should also be a transaction layer that begins at the sensor pack and/or Internet, whoever the originator of the transmission is, that will maintain integrity of communication all the way through the use of the system. This could be implemented as a two phase commit, as current Internet Protocol is designed, or it can be implemented as a four phase commit as described in previously filed patents referenced in the introduction of this patent filing. The gateway/router devices can be implemented in a chain of “grid enabled” devices so that the sensor pack may communicate with the Internet through several gateway/router devices en route during transmission.

Transactions could be used to push logic flow from the Internet to the sensor pack so that the sensor pack is capable of performing some of the logic that would normally be executed on the servers. This could lead to a more distributed computing model for systems based on the “Internet of Things” architecture as described herein. Sensor packs could be used to manipulate robots or perform other actions within products for a number of reasons. One could be for product maintenance. Another could be for product execution, such as running a dishwasher at a scheduled time, or turning on and off lights in a warehouse.

An additional gateway/router design could implement a “long range” wireless transmission protocol such as cellular, satellite, or other communications protocol that would not be considered “short range” or “mid range”, in addition to previously mentioned designs in this and previous filings referenced above. This would be done to wirelessly backhaul data transmissions to the Internet or have the Internet enabled system send transmissions to the gateway/router via a wireless “long range” transmission protocol. The above described gateway/router designs could be used in conjunction with any of the sensor and sensor pack designs mentioned herein.

The “Edge” is the “Internet of Things” (IoT for short) front-line of where technology intersects with business and people, capturing raw data used by the rest of the IoT system. Data is captured by embedding sensors in consumer devices (i.e. fitness trackers, thermostats) appliances or industrial systems (i.e. heating & cooling systems, factory automation) or more specialized applications such as remotely monitoring food temperature and humidity. Such devices can be referred to in this discussion as “Sensor Devices”. Data can then be passed to a “Router” and/or “Gateway” or other “Aggregation Points” that can provide some basic data analytics (parsing raw data) before being sent to the IoT Platform via an Internet connection and beyond. “Routers” can be thought of as local grid or mesh networks whereby implementations such as Bluetooth, Zigbee, WiFi, ANT, OpenWare, LoRa, Sigfox, or other short to mid range wireless transmissions are used to communicate between Sensor Devices and Gateways. Gateways can be thought of as Internet-enabled hardware devices (usually through a wireless WiFi, cellular based such as GSM, CDMA, or other mobile phone carrier network, or landline connection) that communicate either directly to sensors, to sensors through Routers, or a hybrid of both Routers and sensors directly to allow for data to be passed bi-directionally to an Internet platform such as a cloud computing environment or computer network. Also, IoT is not just about capturing data but can also alter the operation of a device with an actuator or other configurable components.

The functionality, shape and size of “Edge” devices are mostly limited by human imagination since most of the technology already exists. For systems including a large number of devices or sensors, gateways and aggregation points serve as the primary connection point with the IoT platform and can collect and prepare data in advance sending the data to the IoT Platform.

### IoT Edge Tier Key Components

### Environment:

This is the operating environment of the sensor or device including natural environments (i.e. outside) or man-made (i.e. buildings, machinery or electronic devices). The environment is important when selecting the sensor to ensure it can withstand the ongoing demands of the environment in addition to power management and maintenance considerations of the “Edge” components.

### Sensors:

This is where the collection of IoT data begins. In most cases the raw data is analog and is converted to a digital format and sent through a serial bus (i.e. I2C) to a microcontroller or microprocessor for native processing. Typical sampling rates for sensors are 1,000 times per second (1 kilohertz) but can vary widely based on need.

### Devices or “Things”:

Sensors are typically embedded within existing devices, machines or appliances (i.e. wind turbines, vending machines, etc.) or in more complex systems such as oil pipelines, factory floors, etc. To eliminate sensors just sending a copious amount of raw data, some of these devices have basic analytical capabilities built-in which allow for some basic business rules to be applied (i.e. send an alert if the temperature exceeds 120 degrees Fahrenheit), as opposed to just sending a live data stream.

### Routers:

A router broadcasts a radio signal that is comprised of a combination of letters and numbers transmitted on a regular internal of approximately 1/10^{th} of a second. They can transmit at this rate, but in an “intelligent” hardware scenario (Intelligent Sensors and/or Routers) the transmission will likely be much slower, as in 5-10 second intervals or exception based as needed. The term “Intelligent” simply means that there is application logic via software and/or firmware that may provide some logic or filtering of sensor data so that transmissions are only sent when conditions are met or a change in sensor data warrants an update to the network. Routers provide an added dimension “Edge” computing with the ability to combine the location of either Bluetooth, WiFi, Zigbee, ANT, OpenWare, LoRa, Sigfox, or other short or mid-range wireless communication protocol equipped mobile devices (i.e. customers) and/or wired devices along with other factors such as current environmental and weather conditions. For example, by tracking the location of devices, more context relevant information can be pushed to the device such as special offers and recommendations based current conditions.

### Aggregation Point or Gateway:

The Gateway or Aggregation Point is the final stop before data leaves the “Edge”. While deploying a gateway is optional, it is essential when creating a scalable IoT system and to limit the amount of unneeded data sent to the IoT platform. Key functions include:
Convert the various data models and transport protocols used in the field, such as Constrained Application Protocol (CoAP), Advanced Message Queuing Protocol (AMQP), HTTP and MQTT, to the protocol(s), data model and API supported by the targeted IoT platform. The HTTP/HTTPS and MQTT are what the gateways will talk to the IoT Platform with. Other local protocols like serial, Zigbee, Bluetooth, WiFi, LoRa, Sigfox, cellular, satellite, and/or OpenWare will normally be used from Router to Gateway.
Data consolidation and analytics (“Edge analytics”) to reduce the amount of data transmitted to the IoT platform so network bandwidth is not overwhelmed with meaningless data. This is especially critical when IoT systems include thousands of sensors in the field.
Real-time decisions that would take too much time if the data was first sent to the IoT Platform for analysis (i.e. emergency shut-down of a device).
Send data from legacy operational technology that may not have the ability to send data to an IoT platform.

When thinking about the technology and design for the “Edge” of an IoT solution, business requirements are more important here than the technology itself, so IT personnel will have to work closely with the business to identify and meet the functionality, costs and security requirements. Once these business requirements are clearly understood does the technology selection process begin (i.e. sensors, gateways and design). At the same time, IT brings insights into the potential and capabilities provided by IoT technology which can help drive use case scenarios so collaboration between the business and IT is essential. After defining the business requirements and the focus has shifted to the technical design of an IoT solution, it is important to first explore any unused IoT infrastructure already built into existing machinery, hardware and software (“Brownfield Opportunity”). There are many types of devices and machines out there already equipped with sensor type technology that is simply waiting to be tapped into. This is the low-hanging fruit that can be quickly leveraged with minimal disruption to the business because the technology has already been adopted while helping accelerate IoT initiatives. The “Greenfield Opportunity” is for IoT opportunities in enterprise environments where no existing IoT infrastructure exists.

There are two major deployment options for “Edge” devices used in an IoT solution:
“Edge” deployment without aggregation
“Edge” deployment with a gateway or aggregation point

### No Aggregation:

Every device is connected to a network (usually the Internet or other IP based system) enabling the device to send and receive data directly to the IoT Platform. This means each device must have a dedicated network and the ability send and receive data using APIs, the data model and transport protocol required by that IoT platform. The device must also have enough computing power for some analytics and to make real-time decisions such as turning off machine if the temperature passes a specified threshold. Finally, the device must have some sort of user interface for maintenance and ongoing updates.

Non-aggregated designs work best when there are few other devices in the area competing for connectivity. Usually, these devices also have more processing power, memory and an operating system capability so it is easier to add or adjust functionality. However, this added device capability is typically more expensive to implement and non-aggregated designs typically don't scale well with each device requiring individual attention to maintain and secure (unless the IoT Platform provides scalable “Edge” device management). Another potential challenge to consider is if the device does not support the IoT platform's transport protocol. In such cases, additional code will need to be added to each device so support the required APIs, data model and transportation protocol.

Aggregation: This design model includes a gateway or some other type of aggregation point connecting “Edge” devices and the IoT platform.

Aggregation designs are ideal for IoT implementations with a large number of sensors, a fleet of devices and where the devices are fixed and localized deployments. This is especially true for scaling and consolidating device management where multiple endpoints can be managed from a single location. Using gateways and other aggregation points in an IoT design allows for cheaper sensors and devices with less computing power while allowing for integration with legacy operational technology that otherwise may not have been available. Gateways can also consolidate the various protocols, data models and APIs from the various end points to the standards required by the IoT platform while also providing a location before data reaches the IoT platform for additional intelligence and intelligence to reduce the amount of data sent to the platform.

However, aggregated designs also provide another layer of complexity into the design by adding gateways or other aggregation points. This essentially means another link in the chain that needs to be monitored and addressed when there are issues. Additionally, without built-in redundancy into the design, this could also lead to a single point of failure when a gateway device goes down and all of the connected devices have no way of communicating with the IoT platform. As a result, all aggregation points must be designed with built-in redundancy.

IoT sensors are basically a monitoring or measuring device embedded into machine, system or device with an API enabling it to connect and share data with other systems. However, sensors can create copious amounts of data which may have no practical value so analytics or exception based models are applied to reduce it to more of a meaningful dataset before transmission. Data is typically transmitted via an IEEE 802.1 network using an Internet Protocol (IP) to a gateway, router, receiver or aggregation point. The transmission frequency can be real-time streaming, exception-based, time intervals or when polled by another system.

The IoT sensor market is divided into two broad categories. Original Device Manufacturers (ODMs) and Original Equipment Manufacturers (OEMs). ODMs design manufacture the core sensor technology (pressure, temperature, accelerometers, light, chemical, etc.) with over 100,000 types of sensors currently available for IoT solutions. These sensors typically do not include any of the communication or intelligence capabilities needed for IoT solutions so OEMs embed ODM sensors into their IoT devices while adding the communications, analytics and other potential capabilities needed for their specified markets. For example, an OEM who builds a Building Automation IoT application may include various sensor types such as light (IR or visual), temperature, chemical (CO2), Accelerometer and contact.

The ODM marketplace is more consolidates and primarily includes established microelectronics and micro processing incumbents who already have the manufacturing facilities and market share such as ST Microelectronics, IBM, Robert Bosch, Honeywell, Ericsson, ARM Holdings and Digi international. On the flip side, the OEM marketplace more of the Wild West. It includes some of the industry heavyweights but is full of a new generation of startups seeking to capitalize on the IoT market. For example, we have Intel, Fujitsu, Hitachi and Panasonic, in addition to a slew of smaller companies such as Lanner, iWave, Artik, and Inventec. The scope of this paper does not include an in-depth analysis of the ODM and OEM vendor landscape.

The following diagram illustrates the typical layout of an IoT Wireless Sensor Device:

### Current State-of-the-Union

Some of the major factors driving the growth of the IoT sensor market includes the development of cheaper, smarter and smaller sensors.

While the IoT sensor and device markets are exciting, dynamic and enjoying growth, the coming wave of these small, embedded, low-power, wireless and wearable devices still do not is enjoy ubiquitous and universal access to the Internet. Due to current battery constraints and longevity, these devices tend to rely on low-power communication protocols such as Bluetooth Low Energy (BLE) as opposed to the more connected and more power intensive protocols such as WiFi and cellular (GSM, 3G/4G, etc.). As a result, most of these devices require an application layer gateway capable of translating the communication protocols, APIs and data models to transmit to the Internet and IoT platform.

### Future Trends

While the majority of IoT applications have traditionally been focused on driving operational efficiencies and cost savings, over the next 12 months, Gartner forecasts enhanced customer experience and new customer based revenue applications will take the lead in over the next 12 months.

The future growth of IoT sensors will be driven by the growing demand for smart devices and wearables, the need for real-time computing and applications, supportive government policies and initiatives, the deployment of IPv6 and the role of sensor fusion. Sensor Fusion is essential the current and future demands of IoT. Sensor Fusion combines data from multiple sensors in order to create a single data point for an application processor to formulate context, intent or location information in real-time for mobile, wearable and IoT devices. It is basically a setoff adaptive prediction and filtering algorithms to deliver more reliable results such as compensating for drift and other limitations of individual sensors.

By combining the growth projections of IoT (50 billion connected devices and a $7.1 trillion market) with the market focus on IoT sensor capability and performance, IoT sensors will be one of the most dynamic and explosive sectors in the market. There will continue to be new OEMs selling IoT applications but the market will also begin to consolidate as the market matures, communication standards are adopted and through M&A activity.

Baseline Requirements when Selecting a Sensor Device:
Security
   Physical
   Firmware
   Data
   Transmission
Power management
   Battery life
   Recharge Ability
Analytical capability
   Sensors or devices producing large amounts of data or IoT systems using a large number of sensors will need to have analytical capability on the “Edge” to filter and select which data will be transmitted to the IoT Platform and beyond. Without “Edge” Analytics, the sheer volume of data can overload networks, create exorbitant communications costs and generate so much data that it becomes very difficult for it meaningful. Additional analytics will happen at the IoT Platform and enterprise applications using the data.
      Exception based reporting . . . .
Communication protocols
Wireless API
Device Maintenance Requirements . . . .

Information from the “Edge” sensors can be integrated through an Internet enabled platform like an “IoT Platform” such as Microsoft's Azure IoT or Amazon AWS Platform to perform various services for the customer. Such services could also be integrated into a company's Enterprise Resource Planning or Customer Resource Management software to perform additional services such as scheduling a service call for a failing home appliance or notifying technical support that a particular robotic arm on a manufacturing floor is not operating correctly.

The “Edge” tier of an IoT architecture should consider using an application tier protocol for communicating with servers in an IoT Platform via a standard such as IoTivity from the Open Connectivity Foundation, the AllJoyn Framework from the AllSeen Alliance, or any other IoT specific protocol for application architecture. Such protocols will allow for Sensor Devices to be registered with an IoT Platform and then have them communicate one way or bi-directionally with the IoT Platform during operation. The “Edge” tier can also be integrated into a Device Manager service on the IoT Platform tier so that Sensor Devices, Routers, and/or Gateway Devices can be observed and managed on the IoT architecture. This will provide availability support so that all devices utilized on the “Edge” tier of the IoT architecture can be monitored and serviced as needed.

The OpenWare wireless mid-range protocol has been enhanced to be more power efficient than other short range wireless protocols such as Bluetooth, Zigbee, ANT and other short range wireless protocols. One such enhancement is to send the body or raw data from the sensor along with the initial wakeup request on the network so that the relevant sensor data is sent in the initial transmission sequence along with the wakeup indication to initiate a transaction. This should require that the sensor data also be encrypted and/or obfuscated so that sensitive information cannot be intercepted during transmissions.

The Sensor Device, Router and Gateway hardware is further enhanced so that sensors such as temperature, pressure, accelerometer, or any other sensor can be remotely calibrated wirelessly. This calibration is a key differentiator as no other Sensor Devices currently support remote calibration of the sensors on-board. These capabilities are in addition to features of the hardware product line mentioned in previously filed disclosures as well as in the hardware/sensor configurations listed below:

Sensor Device Options (SD-Sensor type): Capable of monitoring ID and sensor readings with battery condition, reporting any changes at a preprogrammed time interval. These sensor packs are able to “send” data (transmitter) to the Intelligent Routers and/or Intelligent Gateways for forwarding to either the Local host, Intranet or Internet database via IoT Platform. All models are available with a non-rechargeable coin cell battery offering 400 hours of continuous use, or with a rechargeable battery offering 250 hours of continuous use in the same form factor. All device options have a standard transmission range of 2000 feet line on wireless range with a four-phase commit per transmission to guarantee delivery.

What is mean by “Intelligent” hardware such as Intelligent Sensor Packs/Devices, Intelligent Edge Routers and Intelligent Edge Gateways is that they have their own processor and memory, and can process complex logic on data as it is received and/or stored and/or transmitted. This also means that they support reprogramming the hardware remotely as well as remotely reconfigurable.

Detailed Interpretation of the International Organization for Standardization (ISO) Greenhouse Gas Standard 14064-3

In recent years, energy efficiencies have been promoted and in some cases mandated in various mechanisms. One such effort is the creation of carbon credits. To further explain carbon credits, consider the following excerpt from Wikipedia:

“A carbon credit is a generic term for any tradable certificate or permit representing the right to emit one ton of carbon dioxide or the mass of another greenhouse gas with a carbon dioxide equivalent (tCO2e) equivalent to one ton of carbon dioxide.

Carbon credits and carbon markets are a component of national and international attempts to mitigate the growth in concentrations of greenhouse gases (GHGs). One carbon credit is equal to one ton of carbon dioxide, or in some markets, carbon dioxide equivalent gases. Carbon trading is an application of an emissions trading approach. Greenhouse gas emissions are capped and then markets are used to allocate the emissions among the group of regulated sources.

The goal is to allow market mechanisms to drive industrial and commercial processes in the direction of low emissions or less carbon intensive approaches than those used when there is no cost to emitting carbon dioxide and other GHGs into the atmosphere. Since GHG mitigation projects generate credits, this approach can be used to finance carbon reduction schemes between trading partners and around the world.

There are also many companies that sell carbon credits to commercial and individual customers who are interested in lowering their carbon footprint on a voluntary basis. These carbon offsetters purchase the credits from an investment fund or a carbon development company that has aggregated the credits from individual projects. Buyers and sellers can also use an exchange platform to trade, which is like a stock exchange for carbon credits. The quality of the credits is based in part on the validation process and sophistication of the fund or development company that acted as the sponsor to the carbon project. This is reflected in their price; voluntary units typically have less value than the units sold through the rigorously validated Clean Development Mechanism.”

### Introduction to ISO 14064-3

ISO 14064-3 is the specification and guidance for validation, verification and certification. This international standard is expected to benefit stakeholders worldwide by providing clarification for monitoring, reporting and verifying greenhouse gases. This standard will enhance the environmental integrity of GHG (greenhouse gas) quantification. Greenhouse gases are defined as the following; Common GHGs include Carbon dioxide (CO2), Methane (CH4), Nitrous oxide (N2O), Hydrofluorocarbons (HFCs), Perfluorocarbons (PFCs) and Sulphur hexafluoride (SF6). This specification will also enhance the credibility transparency and consistency of GHG accounting and reporting. This includes GHG project of mission reductions and removal enhancements. The specification will also facilitate the development and implementation of GHG management strategies and plans as well as projects. It will allow entities to track performance and progress in reducing GHG emissions, space as well as facilitate the crediting and trade of GHG emission reductions or removal enhancements.

Potential benefits and applications may include corporate risk management, voluntary initiatives, GHG markets, as well as regulatory and government reporting.

ISO 14064 Greenhouse gases—Part 3: specification and guidance for validation, verification and certification. This document provides guidance and overall requirements for those involved in GHG information validation and verification. Potential users may include GHG validators and verifiers, organizations and individuals involved in developing and commissioning GHG projects, organizations implementing GHG management programs, organizations needed to conduct internal audits of their GHG information, investor, finance, and insurance communities, regulators involved in emissions trading or removal offset programs, as well as voluntary and mandatory GHG scheme administrators.

This international standard specifies requirements and provides guidance for those managing and conducting GHG information, validation and/or verification. GGG information validation and/or verification is based on a number of principles to ensure the following:
Reported data, information and related material are free from material misstatement, avoid misrepresentation and provide a balanced and credible account.
Reported data, information related material are capable of being dependent upon by intended users to represent faithfully that which they either pledge to represent or could within reason expect to represent.
GHG validation and verification conclusions are sufficient to enable validator's and verifiers working independently to reach similar conclusions and similar cases.
GGG information validation and verification processes use generally recognized methods to enable relevant comparison between reported verification and validation conclusions.
GHG validation and verification reports take account the needs of intended users, describes the verification and validation activities undertaken and the level of assurance being provided.

The verification or validation team shall apply the following principles to verify and validate GHG information:
Transparency: present the verification and validation results in a clear, factual, coherent and neutral manner.
Consistency: ensure that GHG information verification and validation activities are comparable over time.
Ethical conduct: be independent of the activity being verified or validated, and free from conflict of interest or bias.
Independence: be independent of the activity being validated or verified and free from bias and/or conflict of interest.
Do professional care: exercise do professional care and judgment in accordance with the importance of the task performed and the confidence placed by stakeholders.
Fair presentation: reflect accurately and truthfully verification and validation activities, findings, conclusions and reports. Report obstacles encountered during the validation verification process and unresolved, diverging opinions between the verification and validation team, the client, and the responsible party.

### Validation and Verification Requirements

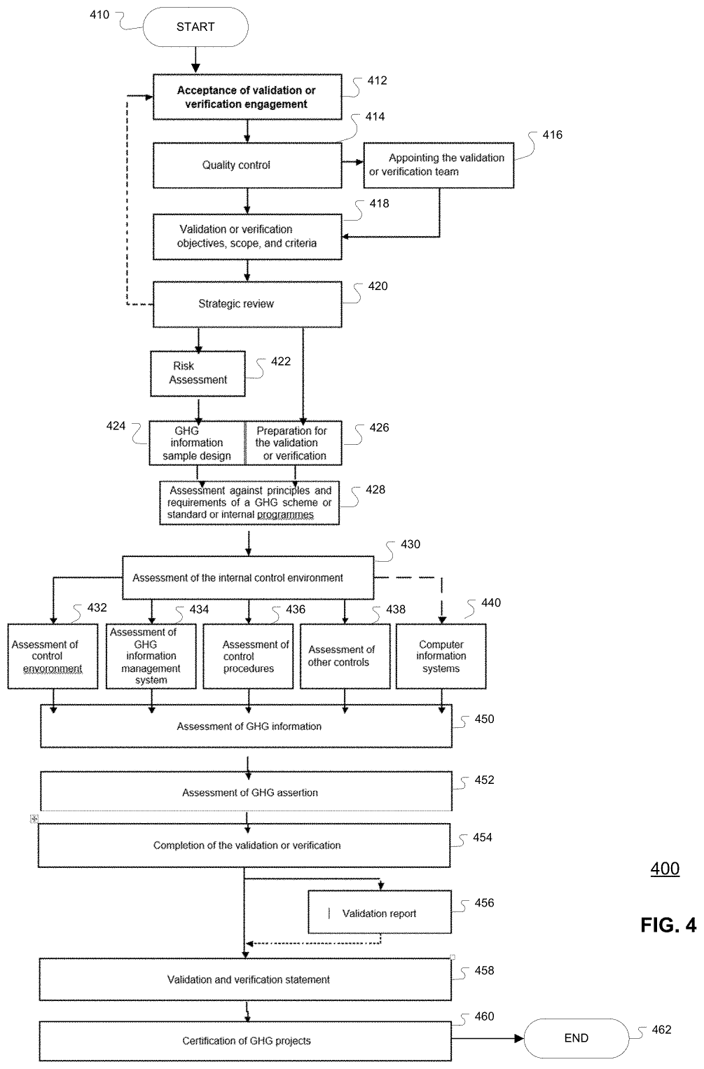

The process of conducting verification and validation is similar, but the nature of the information is different. Validations usually rely on projections and estimates, while verification usually relies on historical information. FIG. 4 shows the process for completing a verification or validation based on ISO 14064-3 requirements.

### Quality Control

The verification and validation team shall implement quality control policies and procedures designed to ensure that its validation or verification work is completed in accordance with the agreed objectives, scope and criteria of the verification or validation, and or relevant GHG requirements and schema principles or standards, as appropriate. The verification or validation team shall communicate quality control policies and procedures to those implementing the verification or validation in a manner in which they are understood and implemented.

### Appointing the Validation or Verification Team

The verification or validation group shall appoint a competent team leader to manage the verification or validation process. The team members and team leader should have skills and competence consistent with their responsibilities and roles. The verification or validation team shall ensure the overall competence of the validation or verification group by the following:
Confirming that the validation or verification team is accredited to operate under any GHG scheme included within the objectives, scope and criteria of the validation or verification where this is a requirement of the GHG scheme.
Identifying the competence, skills and knowledge needed to achieve the goals of the validation or verification plan.
Selecting team members and a team leader with all necessarily competence skills and knowledge represented to achieve the desired goal.

If not entirely represented by the verification or validation team, the necessary competency, knowledge and skills will be provided by unbiased experts. Experts will operate under the direction of the team leader.

The choosing of the verification or validation team shall avoid any potential or actual conflicts of interest with the intended users, responsible party, or client of the GHG information.

### Verification or Validation Criteria, Scope, and Objectives

The verifier or validator and the client shall agree on the verification or validation criteria, scope, objectives and level of assurance required at the beginning of the verification or validation process. The criteria is considered to be what is assessed, the level of assurance is the depth of examination of the evidence, and the scope of the physical and/or temporal boundaries.

### Objective of GHG Project Validation

The objective of GHG project validation is to enable the validating party to express a conclusion on the conservative nests, consistency, completeness and accuracy of the responsible parties GHG assertion(s). The validator shall assess the probability that implementation of the plan GHG project will result in GHG emissions reductions as stated or claimed by the responsible party. The validator shall take into consideration of:
Conformance with applicable validation criteria including the requirements and principles of applicable GHG schemes or standards within the scope of validation.
The documentation and justification of the GHG project plan, including the determination of the baseline, description of the project, project and baseline quantification procedures estimation of GHG emission reductions, as well as quantity, monitoring and reporting procedures or plans.

### Objective of GHG Project Verification

The objective of GHG project verification is to enable the verifier to express a conclusion on the conservative nests, consistency, completeness and accuracy of the responsible party's GHG assertion(s). The verifier shall take into consideration the following:
Conformance with applicable verification and validation criteria including the principles and requirements of applicable GHG standards or schemes within the scope of validation.
The implementation of the GHG project plan and GHG project performance including the following:
   Project and reporting period details including start dates, durations and end dates.
   Description of the project.
   Project baseline determination.
   GHG emissions and removals.
   GHG emissions reduction or removal enhancements.
   Quality monitoring procedures or plans.
   Uncertainty assessment.
   The reporting of project performance including reference to the stated estimations and aims contained within the project plan.
Any relevant changes to the GHG project plan since the last reporting period or since project validation
any relevant changes in baseline and project emissions, emission reductions, removals and removal enhancements since the last reporting period or since project validation.
The effectiveness of GHG related internal controls.

### Objective of Organizational GHG Verification

The objective of organizational GHG verification is to enable the verifier to express a conclusion on the accuracy, completeness, consistency and transparency of the responsible party's GHG assertion(s). The verifier shall take into consideration the following:
the organization's GHG inventory including its GHG removals and emissions.
Any significant changes to the organization's GHG inventory since the last reporting period.
The effectiveness of actual GHG related internal controls.
Conformance with applicable verification criteria including the requirements and principles of relevant GHG standards or schemes within the scope of verification.

### Scope of Validation and Verification

The scope of the validation or verification shall be agreed to by the client. The verification or validation scope shall describe the boundaries and extent of the verification or validation process including the following:
GHG project or organization physical infrastructure, technologies, processes and activities.
GHG project or organization boundaries including operational, geographic, legal and financial boundaries.
Types of GHG's to be included.
GHG sources are sinks to be included.
The frequency of any subsequent verification process required during the organization or GHG project's GHG program.
The time period to be covered.
GHG sources or sinks to be included.
The intended audience, timing and intended use(s) for the validation report and the verification or validation statement.

### Criteria of Validation and Verification

The criteria of the verification or validation shall be agreed to by the client. The verification or validation criteria shall conform to the principles and requirements of relevant GHG standards or schemes within the scope of verification or validation. Validation or verification criteria may include GHG performance targets as well as eligibility requirements.

### Strategic Review

The verifier or validator shall conduct a strategic review of the GHG project's or organization's GHG information to assess the likely nature, complexity and scale of the verification or validation activity to be undertaken on the user's behalf. The strategic review should also consider the level of risk associated with material misstatement, uncertainty, error or omission in the responsible parties GHG assertion(s) and information. The strategic review should be carried out prior to commencing the verification or validation in order to enable an effective verification or validation plan to be designed. The strategic review process shall include an initial evaluation of the GHG projects or organizations GHG assertions and information including a determination that the GHG information provided by the responsible party is a complete representation of the GHG removals, emissions, emission reductions and/or removal enhancements. The verification or validation team leader shall inform the responsible party if the initial evidence presented by the responsible party is found to be inadequate. The team leader may then request further information until the verifier or validator is able to proceed with the verification or validation. The verifier or validator shall postpone or suspend the validation or verification evidence to permit verification or validation to proceed.

### Strategic Review of the Validation of GHG Projects

The strategic review for the validation of GHG projects will include a review of the following documentation and information:
Requirements and principles of GHG standards or schemes to be met by the GHG project, including any quantitative predetermined requirements such as performance targets or materiality thresholds.
The responsible parties GHG assertion(s).
Control and operational procedures to be implemented by the responsible party to ensure security, integrity, and quality of its GHG information.
The GHG project plan including the components listed in preceding section “Objectives of GHG Project Validation”.
Language, social or cultural issues that may impact the execution of an effective validation.

### Strategic Review of the Verification of GHG Projects

### The Strategic Review for the Verification of GHG Projects

The strategic review for verification of GHG project shall include a review of the following documentation and information:
Requirements and principles of GHG schemes or standards to be met by the GHG project including any predetermined quantitative requirements such as performance targets or materiality thresholds.
The responsible parties GHG assertion(s) as well as related previous assertion(s).
Significant changes to the GHG project plan since last verification and or since the validation including any changes to geographic, operational, legal and/or financial boundaries.
The GHG project plan including the components listed in preceding section “Objectives of GHG Project Validation”.
Previous statements and validation reports, certifications or verification statements.
The GHG project statement and validation report including the level of assurance provided.
The GHG project information or report including the following:
   Project GHG removals and omissions quantification procedures.
   Reporting period and project start dates, durations and end dates.
   Baseline GHG removals and omissions quantification procedures.
   Project GHG removals and omissions including appropriate raw data.
   GHG removal enhancements and emissions reductions quantification procedures.
   Baseline GHG removals and omissions including appropriate raw data.
   Baseline and project GHG sources or sinks not subject to regular quantification or monitoring procedures.
   GHG removal and emission reductions enhancements quantification procedures.
   Monitoring and quality procedures or plans.
   Uncertainty assessment.
GHG information management system processes used to process, analyze, gather, collate, transfer, correct or adjust aggregate or disaggregate and store the responsible party's GHG information.
Processes used to gather and review any documentation that supports the GHG information provided.
The control and operational procedures implemented by the responsible party to ensure the security, integrity and quality of its GHG information.
Social, cultural or language issues that may affect the execution of an effective verification.
Evidence of any changes introduced as a result of recommendations from previous verifications or validations.
Reports containing statements on project GHG removals, emissions, emission reductions or removal enhancements related to the responsible parties GHG assertion(s).

### Strategic Review of Verification of Organizational GHG Information

The strategic review for the verification of organizational GHG information shall include a review of the following information and documentation:
a) The organization's GHG assertion(s) and related previous assertion(s);
b) Principles and requirements of GHG schemes or standards to be met by the organization, including any pre-determined quantitative requirements such as materiality thresholds or performance targets;
c) Previous verification reports, statements or certificates;
d) Significant changes to organizational or GHG emissions or removal boundaries since the last verification period, including any changes to legal, financial, operational or geographic boundaries;
e) The GHG inventory or information, including:
   i. Description of the organization;
   ii. Period covered inventory or information;
   iii. Description and justification of organizational boundaries;
   iv. Gross direct GHG emissions separately quantified for each facility, GHG source and type;
   v. Gross GHG removals separately quantified for each facility and GHG sink (if applicable);
   vi. Description and justification for the estimation or exclusion of any GHG source or sink;
   vii. Gross indirect GHG emissions associated with the import or purchase of electricity, heat, steam or other fossil fuel-derived energy products separately for each type of GHG (if applicable);
   viii. Gross indirect GHG emissions associated with other non-energy activities separately quantified for each type of GHG (if applicable);
   ix. GHG emission reductions or removal enhancements from GHG projects quantified within organizational boundaries (if applicable);
   x. GHG emission reductions or removal enhancements from GHG projects quantified outside organizational boundaries (if applicable);
   xi. Description and justification of the base year selected or any change to the base year selected (if established);
   xii. The base year GHG inventory (if established);
   xiii. Description and justification for any adjustment to the base year GHG inventory, including application of the base year GHG inventory adjustment policy (if established);
   xiv. Description and justification of GHG emissions and removals quantification methodologies;
   xv. Description and justification of any change to GHG emissions and removals quantification methodologies previously used;
   xvi. Description and justification for the selection of GHG emissions factors.
f) The operational and control procedures implemented by the organization to ensure the quality, integrity and security of its GHG information;
g) GHG information management system processes used to gather, collate, transfer, process, analyze, correct or adjust, aggregate (or disaggregate) and store the organization's GHG information;
h) Processes used to gather and review any documentation that supports the GHG information provided;
i) Evidence of any changes introduced as a result of recommendations from previous verifications;
j) Language, cultural or social issues that may affect the execution of an effective verification;
k) Reports containing statements on GHG emissions, removals, emission reductions or removal enhancements related to the organization's GHG assertion(s).

### Risk Assessment

The validator or verifier shall conduct a risk assessment. The categories of risk considered shall be:
a) Inherent risk: The inherent risk of a material misstatement occurring;
b) Control risk: The risk that the organization's or GHG project's internal controls will not prevent or detect a material misstatement;
c) Detection risk: The risk that any material misstatement that has not been corrected by the organization's or GHG project's internal control will not be detected by the validator or verifier.

The validator or verifier shall use the information compiled in the strategic review to assess inherent and control risk. Inverse relationships among inherent, control and detection risks shall be used to determine the nature, extent and timing of the sample design and substantive procedures.

### GHG Information Sample Design

The validator or verifier shall develop a sample design. The sample design shall be based on the results of the risk assessment completed as part of the strategic review, in particular where a material misstatement is likely to cause a material impact and the organization or GHG project's control environment and internal control procedures are unlikely to detect and correct the misstatement.

The sample design shall take account of:

a) Validation or verification scope;

b) Level of assurance agreed to with the client;

c) Validation or verification criteria;

d) Results of the strategic review, including the risk assessment.

The sample design shall be amended based on any new risks or material concerns identified throughout the validation or verification process.

### Preparation for the Validation or Verification

The validation or verification team leader shall plan the validation or verification work such that it can be conducted in an effective and timely manner.

The validation or verification team leader should develop and document the validation or verification plan describing:

a) The expected objectives, scope and criteria of the validation or verification, including the risk, materiality thresholds and level of assurance that the client requires;

b) All validation or verification activities to be undertaken, including a description of the type, timing and extent of planned GHG testing methodologies;

c) The GHG information sampling design.

The overall validation or verification plan should be revised as necessary during the course of the validation or verification process.

The extent of validation or verification planning may vary according to the:

a) Size or complexity of the organization or GHG project;

b) Validation or verification team's experience and knowledge of the organization or GHG project,

c) Complexity of the validation or verification;

d) Industrial sector;

e) Technology or processes used.

The validation or verification team leader shall ensure effective communication with the client's management and/or, where appropriate, those responsible for the GHG inventory or GHG project in order to:

a) Confirm the validation or verification plan, including the objectives, scope and criteria of the validation or verification;

b) Describe to the client how validation or verification activities will be undertaken;

c) Confirm communication channels;

d) Provide an opportunity for the client to ask questions.

NOTE—In verification situations, an opening meeting is often used for this communication.

### Assessment Against Principles and Requirements of a GHG Scheme or Standard or Internal Programmes

Where the objectives, scope and criteria of the validation or verification include reference to a GHG scheme or standard, the validator or verifier shall, as appropriate, confirm and determine that the organization or GHG project:

a) Is eligible to participate in the GHG scheme or standard;

b) Will or has used GHG estimation, quantification, monitoring and reporting approaches and methodologies that are approved by, or meet the requirements of, the GHG scheme or standard;

c) Will or has met the GHG performance requirements or targets specified by, or agreed with, the GHG scheme administrators or required by the standard;

d) Will or has reported GHG information that is complete, consistent, accurate and transparent;

e) Has an adequate understanding of the principles and requirements of the GHG scheme or standard and are competent to conform to them;

f) Has specified a level of assurance through the client that is consistent with the principles and requirements of the GHG scheme or standard;

g) Has justified and documented any significant changes to organizational or GHG project boundaries that may lead to a significant or material change in the organization's or project's GHG emissions, removals, emission reductions or removal enhancements since the previous validation or verification period and may affect the organization's or GHG project's ability to conform with the principles, requirements or GHG performance targets of the GHG scheme.

Where the organization or GHG project is seeking entry into a GHG scheme that includes specific entry requirements, the validator or verifier [shall] [should] seek proof that the organization or GHG project has been registered or meets the registration criteria for the GHG scheme. In such cases, the validation or verification body should ensure that it is familiar with its roles and responsibilities in securing registration for the organization or GHG project under the GHG scheme.

Where the objectives, scope and criteria of the verification includes reference to the organization's internal GHG programmes or performance targets, the validator or verifier shall, as appropriate, confirm and determine the:

a) GHG programme follows the organization's documented policies, procedures and codes of conduct;

b) Organization's performance against any target(s);

c) Organization's management and staff have an adequate understanding of the objectives and targets of the GHG programme;

d) Level of assurance specified by the client is consistent with the aims of the organization's GHG programme;

e) Organization has justified and documented any significant changes to organizational or GHG emissions or removal boundaries that may affect the organization's ability to conform with its internal GHG programmes.

### Assessment of the Internal Control Environment

An assessment of the organization's or GHG project's internal control environment shall be undertaken to ensure that:

a) The risk of non-conformance or deviations from principles or requirements of the GHG scheme or standard and/or stated performance targets is minimized;

b) To determine the strategy for detailed testing of GHG information.

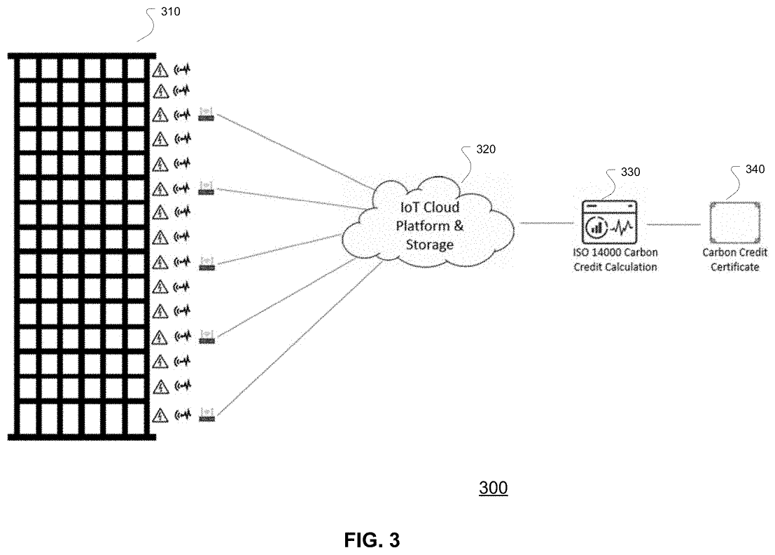

The internal control environment consists of three elements that require simultaneous assessment (FIG. 3):

a) Control environment (Management's philosophy, awareness, roles, responsibilities, authority, and external influences),

b) GHG information management and associated systems (Policies and procedures for recording sources, sinks, and transactions);

c) Control procedures (Specific error checking routines performed by the organization or GHG project).

In the case of GHG project validation, assessing the effectiveness of internal control 966 environments may be practically limited to what is planned, as the validation activity is usually performed before the GHG project has been implemented. When validating GHG projects, the validator shall assess the effectiveness of documented internal control environments as stated in the GHG project plan or through how GHG scheme registration requirements were met.

### Assessment of Control Environment

The control environment influences, but does not ensure, the operational effectiveness and efficiency of the GHG information management system and the application of control procedures.

The validator or verifier shall use information on the organization's or GHG project's control environment to help them plan the GHG validation or verification. The validation or verification team shall assess the effectiveness of the organization's or GHG project's control environment in minimising the risk of errors or omissions within the GHG information system considering:
a) Management's directions, philosophy, awareness and operations in relation to GHG information and reporting;
b) The organization's or GHG project's approach to assigning roles and responsibilities within its GHG programmes and/or GHG projects, including confirmation of individual and collective competency and availability of time and resources necessary for effective and timely monitoring and reporting;
c) External influences that may affect management's behaviour in relation to GHG information and reporting.

### Assessment of GHG Information Management System

The validation or verification team shall assess the effectiveness of the organization's or GHG project's GHG information management system in minimising the risk of errors or omissions within the GHG information system considering:
a) Methods of data or information collection and the relative reliance placed on GHG data or information from different sources;
b) Processes for selecting and implementing GHG estimation and quantification methodologies;
c) Processes for processing, aggregating or disaggregating GHG data;
d) Processes for reporting and disseminating GHG information;
e) The completeness, consistency, accuracy and transparency of the GHG information management system.

FIG. 4 shows the components of a GHG information system, including data handling and the operations that may be used to convert GHG data to GHG information.

### Assessment of Control Procedures

The validation or verification team shall assess the effectiveness of the organization's or GHG project's control procedures in minimising the risk of errors or omissions within the 1011 GHG information system considering:

a) The robustness of the GHG information management system;

b) The completeness, consistency, accuracy and transparency of data collection and input into the GHG information management system;

c) The selection and application of selected GHG quantification methodologies;

d) The appropriateness of aggregation or disaggregation methodologies;

e) Provisions related to staff training and competency, resources and infrastructure;

f) Reconciliation processes for different facilities or GHG projects using different data management approaches to collate, transfer, process, analyze, aggregate, disaggregate, adjust and store their GHG information (where applicable).

### Assessment of Other Controls

Elements of the internal control environment may exist in conjunction, or integrated, with other related controls. Other related controls, including controls within any existing financial, business or environmental management systems, may influence the management and operation of the organization's or GHG project's physical stock, machinery, technologies, infrastructure and processes.

The validation or verification team shall assess the effectiveness of the organization's or GHG project's other controls in minimizing the risk of errors or omissions within the GHG information system considering:

a) Documentation of organizational or GHG project operational or GHG emissions or removal boundaries, including any changes to legal, financial, operational or geographic boundaries;

b) The completeness of the organization's or GHG project's internal controls and operational procedures in relation to the scope of the validation or verification and reported GHG information and assertion(s), including the relationship between any existing financial, business or environmental management systems and internal controls;

c) The organization's or GHG project's internal controls and operational procedures, processes and technologies and the implementation and/or usage of these over the verification period, including the effectiveness of internal calibration, monitoring and verification plans or procedures and their appropriateness to the nature, scale and complexity of GHG-related operations or GHG projects;

d) Records of the organization's or GHG project's performance relating to its control procedures, processes and technologies, including any reported non 1046 conformances, incidents, accidents or reported emergencies during the period of the validation or verification;

e) Relevant non-conformances, incidents, accidents or reported emergencies during the previous validation or verification period, including whether they have re-occurred;

f) Any changes in control procedures, processes and technologies since the validation or previous verification, including their likely impact on the level of risk and materiality issues relating to the GHG information, GHG information management system or associated control environments;

g) Any changes in the organization's or GHG project's product/service mix or ranges that may have a material effect on the responsible party's GHG information and assertion(s).

### Computer Information Systems

Where the GHG information management system exists in part, or as a whole, as a computer information system, the following shall be considered in addition to the requirements in previous sections.

### Systems Risks

The validation or verification team shall consider:

a) Significant risks to the completeness, consistency, accuracy and transparency of reported GHG information from actual or potential failures in the computer information system environment;

b) Breaches of information security that may lead to failures or increased risk in the collation, transfer, processing, analysis, aggregation, disaggregation, adjustment and storage in reported GHG information;

c) Relationships between different computer information systems, including potential affects on GHG information quality and integrity.

### Application Risks

The validation or verification team shall consider:

a) Potential spreadsheet function, software coding, computer scripting or other errors that may lead to significant errors, omissions or material misstatements in the reported GHG information;

b) Potential human errors in the computer information system environment.

### Assessment of GHG Information

Using the sample design and substantive procedures to optimise the validation and verification process and to test the GHG information, the validation and verification team shall assess the organization's or project's GHG information considering:

a) The completeness, consistency, accuracy and transparency of the GHG 1084 information, including raw data origins,

b) The appropriateness of selected GHG estimation and quantification 1086 methodologies;

c) The appropriateness of selected GHG baseline quantification methodologies (if applicable);

d) Whether different facilities or GHG projects (where more than one project is being assessed within the same validation or verification scope) are using different data management approaches to collate, transfer, process, analyse, aggregate, disaggregate, adjust and store their GHG information and how these differences are handled in the GHG information reporting process;

e) The crosschecking of GHG information through other quantification methodologies;

f) Uncertainties in the GHG information arising from different data sources or GHG quantification methodologies;

g) The accuracy and uncertainty of GHG information where a GHG scheme specifies a materiality threshold to which the GHG assertion must adhere;

h) If applicable, the maintenance and calibration programme for equipment used to monitor and measure GHG emissions or removals, including confirming the accuracy of equipment to meet the required accuracy of reporting and any changes to programme that may have a material effect on the reported GHG information and assertions;

i) Any other factors that are likely to significantly affect the GHG information.

### Assessment of the GHG Assertion

The validation or verification team shall determine whether the reported GHG information reflects the GHG assertion(s) being made based on the assessment of GHG information and control environments. The validation or verification team shall assess the GHG assertion(s) by comparing the organization's or GHG project's GHG-related performance against a range of performance criteria, including:

a) The agreed validation or verification objectives, scope and criteria;

b) The performance of the responsible party against any principles or requirements of the GHG scheme or standard and/or any GHG performance targets it has subscribed to;

c) The level of proof provided by objective evidence gathered during the validation or verification that the organization's or GHG project's GHG assertion(s) reflect actual performance and is supported by complete, consistent, accurate and transparent GHG information.

### Completion of the Validation or Verification

The validation or verification is complete when all activities described in the validation or verification plan have been carried out and the validation or verification working papers and supporting evidence have been submitted to the validation or verification body for consideration and independent decision-making. The completion of the validation or verification shall:

a) Include a closing meeting with the responsible party and/or the client (as appropriate), at which the findings and conclusions of the validation or verification are communicated, including any required corrective actions and a timeframe for their completion;

b) As necessary, obtain final data from the responsible party and/or client relevant to the scope of the validation or verification, including data that have been adjusted for reasons of materiality as a result of the validation or verification process;

c) As necessary, assess the organization's or GHG project's rationale and explanation for differences between the final GHG information submitted to the validation or verification team and any GHG information previously submitted to the validation or verification body;

d) Identify any inconsistencies that the organization or GHG project needs to resolve;

e) Close out any outstanding validation or verification trails or inconsistencies;

f) Ensure that validation or verification working papers and supporting evidence (for example notes, diagrams, calculations, spreadsheets) are complete and ready for review by the validation or verification body;

g) Ensure that decisions made during the validation or verification are clearly documented;

h) Ensure that objective evidence and validation and verification trails are clearly documented and sufficiently support validation or verification objectives and the decisions made by the validation or verification team.

As appropriate, where the final sign-off of GHG information and/or GHG assertions) is subsequent to the closing meeting, for example when the responsible party has undertaken required corrective action, the validation or verification team shall conduct a subsequent review to finalize any validation or verification conclusions and issue the validation or verification statement.

In GHG project validations, all issues may not be resolved until the GHG project has been commissioned or has reached day-to-day operational status. This situation may be reflected in the validation statement in the form of limitations or qualifications that may become invalid once the GHG project has achieved operational status.

### Validation Report

The validator shall produce, and is responsible for, a validation report following the completion of the validation. The validation report shall be addressed to the responsible The content and delivery of the validation report shall be agreed by the responsible party and validator and should be based on the agreed scope, objectives and criteria of the validation. The validation report shall provide a complete, accurate and clear record of the validation activities undertaken, and shall, as a minimum, include:
a) Validation objectives, scope and criteria;
b) GHG project plan details, including:
   i) Description of the project;
   ii) Determination of the baseline;
   iii) Project and baseline quantification procedures;
   iv) Estimation of GHG emission reductions and removal enhancements;
   v) Quality, monitoring and reporting plans or procedures.
c) An assessment of the likelihood that the GHG project will achieve estimated GHG emissions reductions or removal enhancements;
d) Identification of the client and responsible party, if different;
e) Identification of the validation team, including the team leader(s) and external experts;
f) Affirmation of the independence of the validators;
g) Validation activities conducted, including their date and place;
h) Findings, including any corrective actions that need to be undertaken by the responsible party;
i) Level of assurance (if provided);
j) Conclusions.

The validation report should be dated and approved in accordance with validation body's quality assurance and quality control procedures.

NOTE 1—Where it is necessary for the GHG project to complete corrective actions, the validation body may issue a draft validation report. The final validation report would be issued when the responsible party had addressed all corrective actions to the satisfaction of the validation body.

NOTE 2: The validation report may be used as a continuous improvement mechanism for future verification activities.

### Validation and Verification Statement

The validation or verification body shall issue a validation or verification statement to the responsible party. The validation or verification statement shall:
a) Be accompanied by the responsible party's GHG assertion;
b) Be addressed to the intended users of the GHG information;
c) Clearly describe the level of assurance provided by the validation or verification consistent with the agreed objectives, scope and criteria of the validation or verification.

The validation or verification statement shall clearly express any circumstance where the validation or verification body:
a) Is of the view that one, some, or all aspects of the GHG information does not conform to the agreed verification or validation criteria;
b) Is of the view that the responsible party's GHG assertion is inappropriate in relation to the agreed validation or verification criteria;
c) Is unable to obtain sufficient, appropriate, objective evidence to assess one or more aspects of conformity of the GHG information with the agreed validation or verification criteria and the responsible party's GHG assertion;
d) Has found it necessary to limit or qualify its opinion.

### Certification of GHG Performance

In some GHG schemes, GHG certification occurs once an independent GHG verifier issues a written assurance that, during a specified time period, an organization or GHG project achieved GHG-performance (for example GHG emissions, removals, emission reductions, removal enhancements) as asserted by the responsible party. The outcome of the certification process is often a formal, written declaration issued by the GHG scheme administrator to the responsible party.

### Annex A

### (Informative)

### Guidance on Validation and Verification Requirements

Annex A provides guidance on the validation and verification requirements contained in the previous sections. Annex A is informative and does not include mandatory requirements.

### Guidance on Validation and Verification Requirements

### Roles and Responsibilities

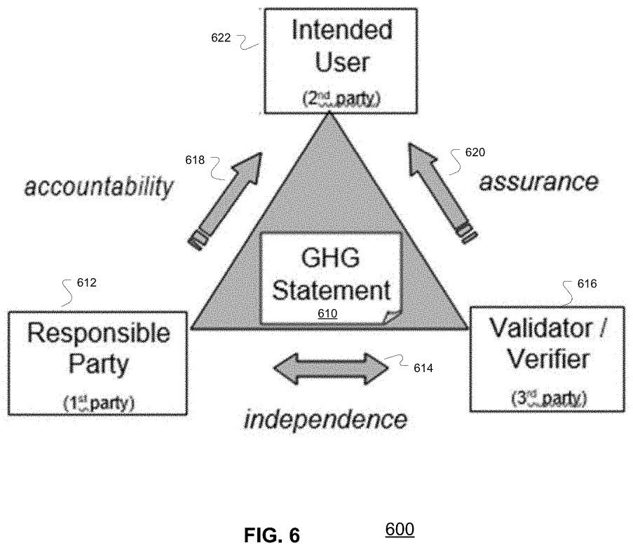

In order to ensure an effective validation or verification, it is important to understand the roles and responsibilities of the parties to the process (FIG. 6). Validation and verification occurs when an impartial validator or verifier objectively evaluates a GHG assertion(s) that has been made by another responsible party—typically the management of an organization or GHG project—against identified and suitable criteria. The validator or verifier then expresses a conclusion that provides the intended user of the information (for example an organization or GHG project), the client (if the two are different) or any other stakeholder likely to be affected by the GHG assertion, with a level of assurance that the GHG assertion(s) is factually correct and contains no errors, omissions or material misstatements. Therefore:
a) The client commissions the validator or verifier to undertake the validation or verification and should ensure that the validator or verifier has sufficient information to determine whether they are capable and competent to conduct the work. It is also the responsibility of the client to agree to the validation or verification objectives, scope and criteria with the validator or verifier;
b) The organization or GHG project (the responsible party) should be responsible for making the GHG assertion(s) and providing it to the objective validator or verifier, along with any information required to support the GHG assertion(s);
c) The validator or verifier should then produce findings and conclusions in the form of a validation report or validation or verification statement, which is distributed to those parties specified in the contract with the client;
d) The intended user of the information may be the client, the responsible party, GHG scheme administrators, regulators, the financial community or other affected stakeholders, such as local communities, government departments or non-governmental organizations. There is an underlying assumption that the intended user is a reasonably intelligent and informed Router of the validation report or validation or verification statement.

### Activities and Decision Points

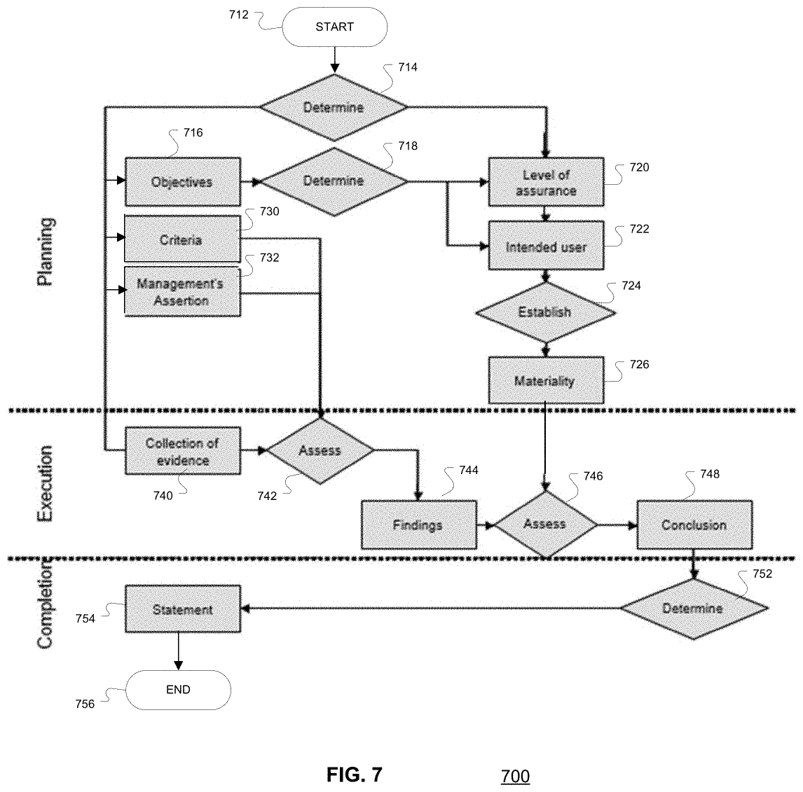

FIG. 7 shows key relationships between activities and decision points in the validation and verification process leading to the issuance of a validation or verification statement.

### Collection of Evidence

Validation and verification activities typically focus on gathering three types of evidence —physical, documentary, and testimonial—by following steps outlined in the validation or verification plan:

a) Physical evidence refers to something that can be seen or touched, such as fuel or utility meters, emission monitors or calibration equipment. Physical evidence is gathered by direct observation of equipment or processes, and is persuasive because it demonstrates that the organization being verified is in the practice of collecting relevant data;

b) Documentary evidence is written on paper or recorded electronically and includes operating and control procedures, log books, inspection sheets, invoices, and analytical results;

c) Testimonial evidence is gathered from interviews with technical, operating, administrative, or managerial personnel. It provides a context for understanding physical and documentary information, but its reliability depends on the knowledge and objectivity of the interviewees.

The more data available, and the more rigorous the review, the more assurance validation or verification will provide. Finding the right approach to validation or verification is largely influenced by the necessary degree of accuracy and credibility (ie, level of assurance) required by the client. For example, companies selling GHG emissions reductions or removal enhancements in an emissions trading or carbon offsets market will require greater accuracy and credibility than companies seeking merely to understand and report on their GHG emissions or removals as part of a voluntary GHG scheme.

Verification testing may include a wide variety of activities, such as retracing data to find omissions or transcription errors, re-computing emissions estimates to confirm engineering calculations, or reviewing documents attesting to an activity.

### Guidance on Quality Control

Quality control measures should include policies and processes for:

a) Ensuring independence (applicable only to third party validations or verifications): For third party validations or verifications, the validation or verification body should have adequate procedures for maintaining and monitoring its independence from the responsible party and/or the client;

b) Assuring the quality of the validation or verification process: The validation or verification body should implement quality control policies and procedures designed to ensure that all validations or verifications are conducted in accordance with this International Standard or other relevant principles or requirements of a GHG scheme or standard, as specified in the validation or verification criteria. The validation or verification body's general quality control policies and procedures should be communicated to its personnel in a manner that provides reasonable assurance that the policies and procedures are understood, implemented and followed;

c) Dispute resolution: The validation or verification body should have procedures and processes in place prior to the commencement of the validation or verification to handle appeals, complaints and disputes brought before it by the client or another affected party. The validation or verification body should be able to demonstrate that, in the functioning of its procedures, the personnel involved in the resolution of such disputes are competent to do so;

d) Distribution and application of validation or verification materials: The validation or verification body should have processes and procedures that ensure that the client and/or the responsible party do not use validation or verification reports and statements or certificates for uses other than for which they are intended and within the principles and requirements of any applicable GHG scheme.

In most instances, the quality control requirements of the validation or verification body may be met through an internal peer review process.

### Guidance on Appointing the Validation or Verification Team

In deciding the size and composition of the validation or verification team, consideration should be given to:

a) The objectives, scope, criteria and estimated duration of the validation or verification;

b) Whether the validation or verification is to be conducted by two or more validation or verification bodies (ie, is it a combined or joint engagement);

c) The overall competence of the validation or verification team needed to achieve the objectives of the validation or verification;

d) The accreditation, certification or legal requirements of any GHG scheme to which the organization or GHG project subscribes;

e) The ability of the team members to interact effectively with the client and the responsible parties and to work together within the agreed scope and objectives of the validation or verification;

f) The language(s) to be used during the validation or verification, and an understanding of the organization or GHG project's particular social, cultural and geographical characteristics.

These issues may be addressed either by the validation or verification body's own skills and competencies or through the support of subcontracted technical or financial expert. Trainee validators or verifiers may be included in the validation or verification team under the direction and supervision of a competent and qualified team member.

NOTE—The principles and requirements of some GHG schemes require an institutional separation of validation and verification activities between two bodies or at least stipulate that different teams from the same validation or verification body conduct the validation and verification activities independently of each other to avoid any potential or actual conflicts of interest.

### Use of Experts

A validator or verifier should use an expert when his or her subject matter expertise is insufficient to appropriately understand and assess significant aspects of the validation or verification.

There are a range of relationships between a validator or verifier and an expert:

a) Independent experts: An independent expert is one who takes instruction from the validator or verifier and provides information and findings. The expert's findings are included in the validator or verifier's working papers and reviewed accordingly. Examples of this type of relationship are the:

i. Validator or verifier uses a report prepared by an expert for another purpose as evidence;

ii. Validator or verifier and the expert perform separate but complementary projects in which the expert's work and findings provide evidence for the validator or verifier;

iii. Validator or verifier engages an expert to perform specific procedures to provide evidence;

b) Team member experts: A team member expert is involved in planning, decisions, completion of the work and consideration of findings. The expert's work and findings are documented as part of the validator or verifier's working papers and reviewed accordingly;

c) Responsible party experts: The validator or verifier can use experts employed by the responsible party; however, the level of assurance provided by a responsible party expert is less than that provided by an independent expert or a team member expert. The responsible party expert's findings are included in the validator's or verifier's working papers and when the validator or verifier assesses the findings, the independence of the expert from management responsible for the GHG assertion(s) should be considered. Because the validator's or verifier's team members need to be independent of the accountable party, it is inappropriate for an expert employed by the responsible party to be a member of the validation or verification team.

In evaluating the expert for a particular validation or verification, the validator or verifier should consider the:

a) Expert's expertise, competence and integrity;

b) Relevance of the expert's expertise to the objective of the validation or verification;

c) Expert's objectivity and appropriate degree of independence in relation to the practitioner's and GHG scheme requirements.

The validator or verifier should be satisfied that there is appropriate understanding between the validator or verifier and the expert on their respective roles and responsibilities.

### Roles and Responsibilities of the Validator or Verifier

The validator or verifier should have the ability to:

a) Understand the objectives of the expert's work and how it relates to the objectives of the validation or verification;

b) Consider and conclude on the reasonableness of the assumptions, methods and source data used by the expert;

c) Conclude on the reasonableness and significance of the expert's findings in relation to the objective of the validation or verification and the validator's or verifier's conclusion.

The validator or verifier needs to determine how much understanding of the validation or verification process, and proficiency in its application, the expert requires based on the expert's role in the project.

### Roles and Responsibilities of the Expert

Experts do not need to understand the validation or verification process and techniques to the same degree as the validator or verifier. However, they do need to understand the objectives and nature of the validation or verification process sufficiently to understand their role and to apply professional standards in the context of their responsibilities.

In relation to using the work of an expert, findings are the output of the work performed by the expert that the validator or verifier uses as evidence. In all cases, the findings and, if necessary, the work of an expert should be reviewed by the validator or verifier. The extent of the review is based on the significance of the expert's involvement, the reliability of the evidence the specialist provides, and the validator or verifier's assessment of the risk that the specialist's findings may be significantly in error.

The validator or verifier should obtain sufficient appropriate evidence concerning the expert's work and findings in order to consider and determine the reasonableness of the:

a) Source data used by the expert;

b) Expert's assumptions and methods and, when applicable, their consistency with those used in prior periods;

c) Expert's findings.

The validator or verifier should conclude on the relevance of the expert's findings in relation to the objective of the validation or verification and the validator's or verifier's overall conclusion on the subject matter.

Before the validator or verifier accepts the expert's work and findings, the validator or verifier needs to exercise professional skepticism and consider the reliability of the evidence the expert provides, based on the validator or verifier's assessment of the risk that the expert's findings may be significantly in error.

### Internal Peer Review

Current best practice includes the appointment of an internal objective peer reviewer at the same time as the appointment of the validation or verification team leader, in order to provide expert oversight of the validation or verification process and outcomes. Best practice also indicates that validation and verification risk can be significantly reduced through the appointment of an objective peer reviewer, who assesses the work of the team leader and the validation or verification team from the initial contact with the client to the completion of the validation or verification process.

When conducted, the peer review process should be conducted throughout the validation or verification process from the appointment of the peer reviewer by the validation or verification body at the beginning of the process to the completion of work, including the decision to issue the validation or verification statement. The purpose of peer review is to ensure that the validation or verification process is conducted with due professional care and judgement and that any verification risks are minimised. Peer review should focus in particular on the following validation or verification activities:

a) Appointment of the validation or verification team leader;

b) Strategic review, including initial risk assessment and materiality analysis;

c) GHG sample design;

d) Validation or verification plan;

e) Selection of team members—including competency evaluation;

f) The assessment of control environments;

g) The draft validation or verification report and statement—including the validation or verification findings and conclusions;

h) Any non-conformities raised by the validation or verification team, particularly those that prohibit an unqualified validation or verification statement;

i) The decision to issue the validation or verification statement.

Should any discrepancies come to light during the peer review process, such as new errors and omissions or outstanding materiality issues, the peer reviewer should refer these issues to the team leader and/or the responsible party as appropriate. Any new material discrepancies identified by the peer reviewer must be rectified to the peer reviewer's satisfaction before the validation or verification body can issue any validation or verification statement or before they certify GHG performance (should this be required as a discrete activity).

### Guidance on Validation and Verification Scope, Objectives and Criteria Level of Assurance

In a validation or verification, the validator or verifier assesses the evidence collected as a result of the process and expresses a conclusion in the form of a validation or verification report. It is important to establish at the beginning of the process the degree of assurance sought by the client and intended user as it will dictate the extent to which the validator or verifier will apply certain procedures to obtain the relative degree of satisfaction that the evidence supports the level of assurance. In general, there are three levels of assurance; high, moderate and none.

For a high level of assurance, the validator provides a high, but not absolute, level of assurance that the management's GHG assertion(s) is free of material misstatement. This is expressed positively in the validation or verification report as reasonable assurance. The validator or verifier provides a high, though not absolute, level of assurance by designing the process and procedures so that in the validator's or verifier's professional judgment, the risk of an inappropriate conclusion is reduced to a low level. Absolute assurance is not attainable as a result of factors such as the use of judgment, the use of testing, the inherent limitations of control and the fact that much of the evidence available to the validator or verifier is persuasive rather than conclusive in nature. Assurance will also be influenced by the degree of precision associated with the GHG estimation or quantification methodology.

For a moderate level of assurance, the validator or verifier provides a moderate level of assurance that the information subject to review is free of material misstatement. This is expressed in the form of negative assurance. The validator or verifier provides a moderate level of assurance by designing the process and procedures so that, in the validator's or verifier's professional judgment, the risk of an inappropriate conclusion is reduced to a moderate level when the evidence obtained enables the validator or verifier to conclude the management's assertion is plausible in the circumstances.

Moderate level validations and verifications are distinguishable from high-level validations and verification in that there is less emphasis on detailed testing in a moderate level validation or verification and procedures used consist primarily of enquiry, analytical procedures and discussions related to GHG information supplied. In moderate level validations and verifications, it is essential that the validator or verifier not lead the intended user to conclude that a high level of assurance is being provided and consequently must use a negative assurance style when expressing a conclusion in the validator or verification report.

The validation or verification report with moderate level of assurance should contain the following basic elements, ordinarily in the following layout:

a) A title;

b) The addressee;

c) An opening or introductory paragraph including:

i. Identification of the management assertions on which validation or verification has been performed;

ii. A statement of the responsibility of management and the responsibility of the validator or verifier;

d) A scope paragraph, describing the nature of the processes applied, including:

i. A reference to this International Standard or other relevant standards or practices;

ii. A statement that a process was limited primarily to inquiries and analytical procedures;

iii. A statement that high level of assurance has not been provided, that the process and procedures undertaken were designed for a moderate level of assurance;

e) A statement of negative assurance;

f) The date of the report;

g) The auditor's address;

h) The auditor's signature.

Assistance with compilation of management's assertion on GHG information is considered to provide no assurance.

### Validation or Verification Criteria

Several parties may set validation or verification criteria, including:

a) Governments may set specific GHG performance criteria as part of national or regional regulatory requirements;

b) GHG emissions trading programmes may contain criteria as part of their eligibility or scheme entry requirements;

c) Voluntary reporting initiatives may have set criteria as part of their participation or scheme entry requirements.

### Guidance on Strategic Review

The strategic review process lays the foundation for validation and verification planning and provides the first real opportunity for the validation or verification team to assess the completeness, consistency, accuracy and transparency of the responsible party's GHG information and GHG assertion(s). Strategic review should include an initial risk assessment and an analysis of any actual or potential failures that are likely to give rise to materiality issues in the responsible party's GHG information and GHG assertion(s).

### Example—Materiality

The objective of any validation or verification of GHG information is to enable the validation or verification body to express an opinion on whether the organization or GHG project's GHG assertion(s) are prepared, in all material respects, in accordance with the intent of their internal GHG programmes or any GHG scheme to which they subscribe. The assessment of what is material is a matter of professional judgement. The concept of materiality recognizes that some matters, either individually or in the aggregate form, are important if the responsible party's GHG assertion(s) are to be presented fairly in accordance with internal requirements or that of the GHG scheme to which it subscribes.

A misstatement or the aggregate of all misstatements in GHG assertion(s) is considered to be material if, in the light of surrounding circumstances, it is probable that the decision of a person who is relying on the GHG assertion(s), and who has a reasonable knowledge of business and GHG activities (the intended user), would be changed or influenced by such misstatement or the aggregate of all misstatements.

Although the validator or verifier is required to determine materiality based on his or her perception of the needs of intended users of the information, it is extremely difficult to predict with certainty who those users will be or, indeed, the specific needs of known users. Consequently, the materiality decision ultimately becomes a matter for the validator or verifier's professional judgement. In order to ensure consistency and avoid unanticipated discrimination, some GHG schemes or internal programmes assist this decision-making process by including materiality thresholds. These may be defined at the overall level, such as 5% of an organization or GHG project's gross direct GHG emissions. They may also include varying thresholds depending on the level of disaggregation, such as 1% at the gross organizational level, 5% at the facility level and 10% at the source level. Further, a series of discrete errors or omissions identified within a particular disaggregation level, individually less than the materiality threshold may, when taken together, exceed the threshold and thus be considered material. Omissions or errors identified that represent amounts greater than the stipulated threshold are pre-determined as being a “material discrepancy”, that is, a non-conformity.

The determination of materiality involves qualitative as well as quantitative considerations. As a result of the interaction of these considerations, misstatements of relatively small amounts could have a material effect on the GHG assertion(s).

### Information and Documentation

In addition to documentation identified in Clause 5.4, validators and verifiers may find it useful to review the following information if available:

a) A GHG information flow diagram;

b) An annotated process flow diagram, characterizing mass or energy flows for selected GHG sources and sinks;

c) A mass balance, energy balance and/or other quantitative balance for selected GHG sources and sinks;

d) Findings from any industry, organization or GHG project internal assessments or audits;

e) Prior GHG validations or verifications that relate to the organization or GHG project's GHG information management process and systems, or the quality of its GHG information;

f) The (non) existence of an operating environmental management system and its application in the quantification, monitoring and reporting of GHG emissions and removals.

### Guidance on GHG Information Sample Design

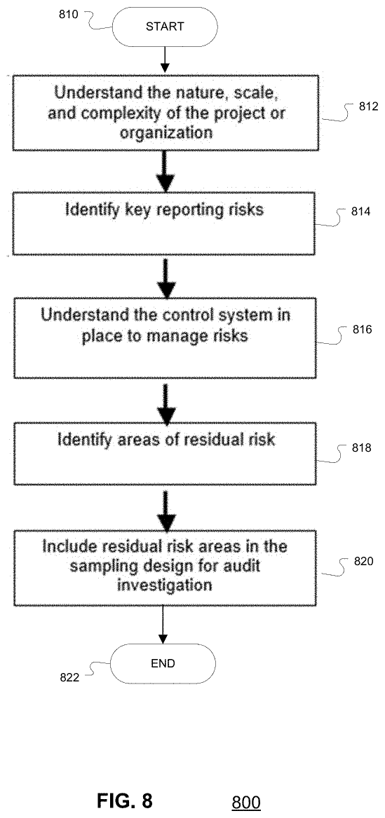

It is generally inefficient to assess all GHG information collected by the organization or project, therefore a risk-based approach should be used to determine the sample design for further audit investigation. Typical steps in a risk based approach follow FIG. 8.

Examples of reporting and control risks include:

a) Incompleteness: for example, exclusion of significant sources, incorrectly defined boundaries, leakage effects

b) Inaccuracy: for example, double accounting, significant manual transfer of key data, inappropriate use of emission factors)

c) Inconsistency: for example, not documenting methodology changes in calculating emissions from those used in previous years)

d) Data management and control weaknesses: for example, no checking of manual transfers of data from the point of origin and between calculation spreadsheets, no internal audit/review process, inconsistent monitoring, no calibration and maintenance of key process parameters/measurements e.g. metering and sampling/analysis

### Example—Risk-Based Approach for Project Validation

The risk based approach for validation should identify the key risks associated with the assumptions made and GHG information used within the:
Project design;
Baseline determination (eg, scenario, methodology, estimation and additionality (when applicable));
Project and baseline GHG quantification procedures;
GHG emission reduction or removal enhancement estimates;
Quality and monitoring plans or procedures;
Environmental impact analysis (if applicable)

The two main sources for uncertainties in estimating GHG emission reductions or removal enhancements from GHG projects are normally:
Baseline uncertainty: There are uncertainties associated with the counterfactual assumptions made for the baseline when projecting a set of circumstances that may never occur (eg, baseline technology/fuel, performance of baseline technology, timing of replacement/length of timeframe, equivalence of services);
Data uncertainty: The technical uncertainties associated with the determination and the measurement of the parameters necessary to estimate the GHG emission reductions or removal enhancements (eg, output, efficiency of plant/networks, emission factor, utilization factor). There may also be accidental reporting errors that are related to human error or problems in the reporting routines.

The baseline potentially creates the greatest uncertainty in the GHG emission reduction or removal enhancement estimates, as it inherently projects a set of circumstances that never occur. The uncertainty associated with the assumptions made for the baseline can never be completely removed. Lacking appropriate means for quantifying this type of uncertainty, the most conservative, yet reasonable baseline should be selected.

In addition, to a risk-based approach there are a number of selection methods that are commonly used in combination to determine the GHG information sampling design. Methods include samples based on:

a) GHG sources;

b) GHG sinks;

c) GHG types:

d) Organizations, facilities, sites;

e) GHG projects;

f) GHG processes.

Sample design should be treated as an iterative process, as the sampling approach or the information samples chosen may need to be changed, as weaknesses in control environments, GHG information and materiality issues are identified during the validation or verification. Revisions to the sample design should consider the sufficiency and appropriateness of evidence from testing methodologies together with any control evidence to support the organization or GHG project's GHG assertions.

### Guidance on Preparation for the Validation or Verification

It is generally inefficient to collect and assess all organization and GHG project GHG information. Therefore, a risk-based approach is used to design the validation or verification plan. The process of designing the validation or verification plan consists of:

a) An initial assessment of the findings of the strategic review process to understand the root causes of any identified or potential GHG information errors, omissions, materiality issues or failures and weaknesses in control environments;

b) Reference and consideration of any previous validation or verifications, and/or comparable validations or verifications of similar organizations or GHG projects;

c) The sample design, including the rationale behind the approach being taken;

d) Identification of the types of potential material misstatements that could occur in the GHG assertion(s);

e) Consideration of risks that could cause material misstatements;

f) Design of appropriate methodologies to test whether material misstatements have occurred or errors or omissions have been made;

g) Amendment of the validation or verification plan throughout the validation or verification process to take account for any new evidence relating to actual or potential errors, omissions, materiality issues and the prevailing performance of the control environment(s).

The risks considered in the validation or verification plan are:

a) Inherent risk;

b) Control risk;

c) Detection risk.

Matters to be considered by the validation or verification team in developing the overall validation or verification plan should include findings from the strategic review and:

a) The validation or verification body's knowledge of the responsible party's business, including:

i. The industry conditions affecting the organization or GHG project's reporting of GHG emissions, removals, emission reductions or removal enhancements and levels of disclosure;

ii. The characteristics of the organization or GHG project, its business, its GHG performance and its GHG reporting requirements, including changes since the validation or the previous verification period;

iii. External reporting requirements for GHG information;

iv. The robustness and maturity of the prevailing control environments;

v. The general level of competence of the organization or GHG project's management and those responsible for the gathering, transferring, processing, analyzing, aggregating, disaggregating, storing and reporting of the GHG information that supports the GHG assertion(s).

b) Understanding the GHG information collection and internal control systems, including:

i. The validation or verification body's cumulative knowledge of a range of different GHG information collection and internal control systems and the relative emphasis expected to be placed on tests of control and substantive procedures according to the approach taken by the responsible party.

c) Risk assessment and materiality analysis, including:

i. The assessment of inherent and control risks; and the potential for detection risks to occur;

ii. The setting of materiality levels for reporting purposes;

iii. The possibility of material misstatement, including the experience of past periods;

iv. Identification of complex GHG quantification requirements (eg, where the use of complicated conversion factors or methodologies are likely to lead to variability in GHG information by the organization or GHG project);

v. Determining access to, and availability of, relevant, recognized and up-to-date external emissions factors.

d) Coordination, direction, supervision and review, including:

i. The number of validation or verification components (eg, the number of facilities, GHGs, manufacturing processes, control environments, computer information systems, subsidiaries, branches and divisions);

ii. The involvement of technical and financial experts and the importance of their contribution to the overall validation or verification process;

iii. Number, roles and responsibilities of team members;

iv. Number of different disciplines and/or competencies required to undertake an effective validation or verification process.

e) Other matters, including:

i. Conditions requiring special attention, such as the existence of third parties, joint ventures or outsourcing arrangements;

ii. The terms of the contract with the client (eg, timescales for delivery) and any GHG scheme responsibilities and competency requirements;

iii. The nature and timing of reports or other communications with the client, the responsible parties or the intended users of the information, including the administrators of any GHG schemes to which they subscribe;

iv. The frequency with which the validation or verification has to be conducted to satisfy internal client requirements, the needs of regulators and other stakeholders and any GHG schemes to which the organization or GHG project subscribes.

### Conducting an Opening Meeting

In many instances, for example internal GHG information validation or verification in a small organization, the opening meeting may simply consist of communicating that a validation or verification is being conducted and explaining the nature of the validation or verification.

For other validation or verification situations, the meeting should be formal and records of attendance should be kept. The validation or verification team leader should chair the meeting and the following items, as appropriate, should be considered:

a) Introduction of the participants, including an outline of their roles;

b) Confirmation of the validation or verification objectives, scope and criteria;

c) Confirmation of the validation or verification timetable and other relevant arrangements with the client and/or the responsible party, such as the date and time for the closing meeting, any interim meetings between the validation or verification team and the client's management, and any late changes;

d) Methods and procedures to be used to conduct the validation or verification, including advising the client that the validation or verification evidence will only be based on a sample of the information available and that therefore there is an element of uncertainty in the validation or verification;

e) Confidentiality issues and procedures;

f) Confirmation of formal communication channels between the validation or verification team and the client and/or the responsible party;

g) Confirmation of the language to be used during the validation or verification;

h) Confirmation that, during the validation or verification, the client and/or the responsible party will be kept informed of validation or verification progress;

i) Confirmation that the resources and facilities needed by the validation or verification team are available;

j) Confirmation of information and data access;

k) Confirmation of relevant site access, work safety, emergency and security procedures for the validation or verification team;

l) Confirmation of the availability, roles and identities of any guides;

m) The method of reporting, including any grading of nonconformities;

n) Information about conditions under which the validation or verification may be terminated or suspended;

o) Information about any appeal system on the conduct or conclusions of the validation or verification;

p) Opportunity for the responsible party to ask any questions.

### Guidance on Assessment of Control Environment

A wide variety of activities characterize the organization's or GHG project's control environment. Key aspects are discussed below.

### Management Philosophy and Operating Style

Management philosophy and operating style covers a broad range of characteristics which include:

a) Integrity and ethical values;

b) Approach to taking and monitoring GHG risks;

c) Attitudes and actions concerning GHG reporting;

d) Emphasis on meeting GHG and other related financial and operating goals.

These characteristics significantly influence the control environment, particularly when one or a few individuals, regardless of other control environment factors, dominate management.

### Methods of Assigning Authority and Responsibility

Methods of assigning authority and responsibility affect the understanding of GHG reporting relationships established within an organization or GHG project. Information to consider includes:

a) Organization or GHG project policy on matters such as acceptable business practices, conflicts of interest and codes of conduct;

b) Assignment of responsibility and delegation of authority to deal with matters such as organizational goals and objectives, operating functions and regulatory requirements;

c) Employee job descriptions specifying duties, reporting relationships and constraints;

d) Computer systems documentation indicating procedures for authorizing GHG transactions and approving systems changes.

### Management Control Methods

Management control methods affect management's direct control over the exercise of authority delegated to others and its ability to effectively supervise the organization's activities. Such methods include consideration of:

a) Establishing planning and reporting systems that set forth management's plans and the result of actual performance;

b) Establishing methods that report actual performance and exceptions from planned performance, and communicating this information to appropriate levels of management;

c) Using such methods at appropriate management levels to investigate variances from expectations and to take appropriate and timely corrective action.

### Systems Development Methodology

Systems development methodology involves establishing and maintaining methodologies for developing and modifying systems and procedures, including related computer programs and data files.

### Personnel Policies and Practices

Personnel policies and practices affect an organization's ability to employ sufficient competent people to meet its goals. Specifically, they include policies and practices for hiring, training, evaluating, promoting and compensating employees, and for giving them the resources necessary to carry out their assigned responsibilities.

### Management Reaction to External Influences

External influences are established and exercised by outside forces and affect the organization's operations and practices. Examples include monitoring and compliance requirements imposed by legislative and regulatory bodies. Ordinarily, such influences are outside an organization's authority. However, they may heighten management's consciousness of an attitude toward conducting and reporting an organization's operations. They may also prompt management to establish specific policies and procedures.

### Internal Audit

Internal audit is an activity within the control environment that functions by measuring and evaluating, as a service to the organization, the effectiveness of other activities. Internal auditors are responsible for providing analyses, evaluations, recommendations and other information to the organization's management and board of directors, or to others with equivalent authority and responsibility. In performing these functions, internal auditors are part of internal control.

### Guidance on the Assessment of Control Procedures General Control Procedures

General control procedures consist of how the organization or GHG project determines:

a) Capable personnel: One of the most important aspects of the control procedure is to ensure that capable personnel are performing the work. A high turnover in personnel maybe indicative of a control procedure weakness;

b) Segregation of responsibilities: The segregation of responsibility, or division of duties, is important in ensuring that incompatible responsibilities do not create the need or ability to create or conceal errors;

c) Control access: Access to important records should be limited to authorised personnel;

d) Periodic comparison: Those who do not have responsibility for recording the GHG information should perform periodic comparison.

### Error Checking Routines

There are numerous methods for checking GHG information that can be categorised into input, transformation and output controls (Table A1). Input controls are procedures for checking the data from the measured or quantified values to a hard copy. Transformation controls refer to error checking during the process of collating, transferring, processing, calculating, estimating, aggregating, disaggregating or adjusting input data. Output control refers to control surrounding the distribution of GHG information and comparisons between input and output information.

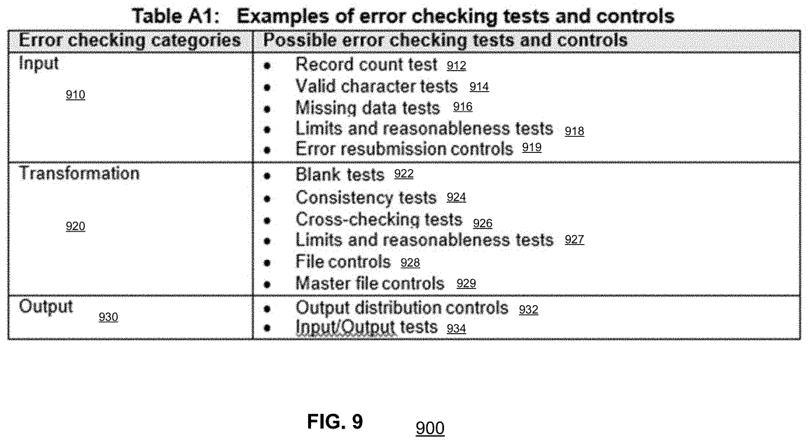

### FIG. 9: Examples of Error Checking Tests and Controls

### Error Checking Categories Possible Error Checking Tests and Controls

### Input

Record count test
Valid character tests
Missing data tests
Limits and reasonableness tests
Error resubmission controls

### Transformation

Blank tests
Consistency tests
Cross-checking tests
Limits and reasonableness tests
File controls
Master file controls

### Output

Output distribution controls
Input/Output tests

### Guidance on Assessment of GHG Information

The degree of inherent accuracy and reliability that can be attributed to GHG information will depend on the data source and the ways in which the GHG information has been collected, calculated, transferred, processed, analyzed, aggregated or disaggregated and stored. The categorization of GHG information sources may help validators or verifiers to understand how much they can depend on the accuracy or reliability of GHG information from different sources.

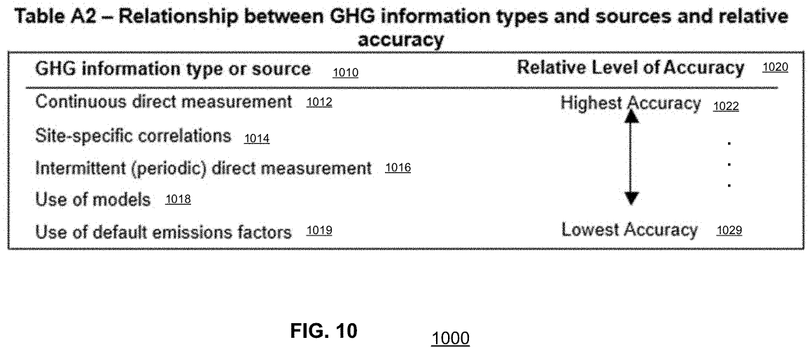

FIG. 10 illustrates the relationship between GHG information types and sources and relative accuracy. There is a tendency for greater accuracy and precision as you advance up the tiers; although this may vary qualitatively depending on the specifics of any given validation or verification. It should be noted that few GHG emissions are directly measured and most are obtained through emissions factors or the use of models.

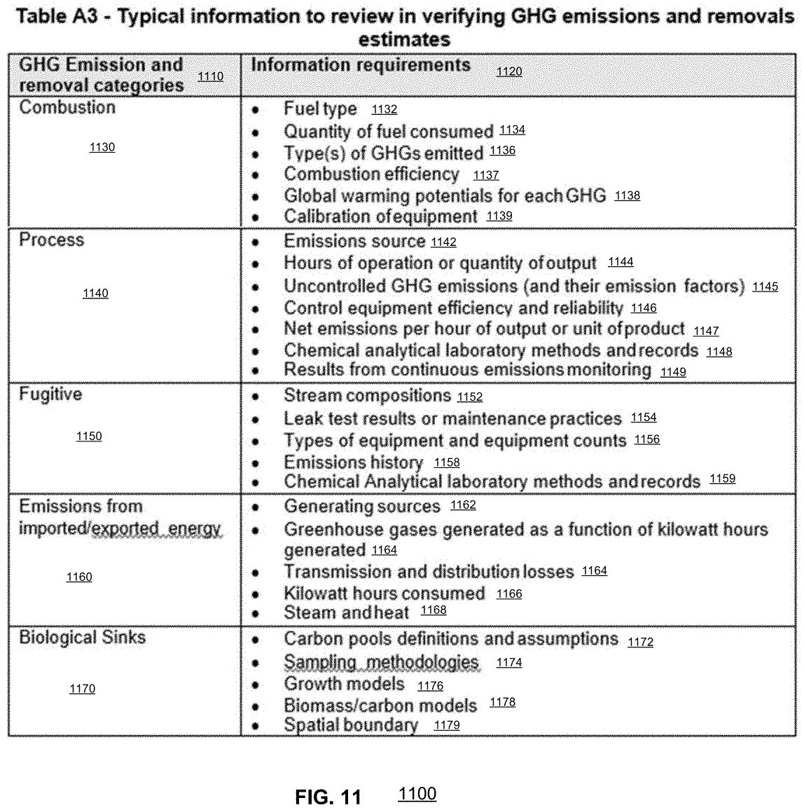

FIG. 11 lists typical information to review in verifying GHG emissions and removals depending on emissions and removal categories and GHG quantification methodologies.

### GHG Emission and Removal Categories Information Requirements

### Combustion

Fuel type
Quantity of fuel consumed
Type(s) of GHGs emitted
Combustion efficiency
Global warming potentials for each GHG
Calibration of equipment

### GHG Emission and Removal Categories Information Requirements

### Process

Emissions source
Hours of operation or quantity of output
Uncontrolled GHG emissions (and their emission factors)
Control equipment efficiency and reliability
Net emissions per hour of output or unit of product
Chemical analytical laboratory methods and records
Results from continuous emissions monitoring

### Fugitive

Stream compositions
Leak test results or maintenance practices
Types of equipment and equipment counts
Emissions history
Chemical Analytical laboratory methods and records
   Emissions from Imported/Exported Energy
Generating sources
Greenhouse gases generated as a function of kilowatt hours generated
Transmission and distribution losses
Kilowatt hours consumed
Steam and heat

### Biological Sinks

Carbon pools definitions and assumptions
Sampling methodologies
Growth models
Biomass/carbon models
Spatial boundary

In addition to checking GHG emission sources under standard operating environments or normal conditions, validators and verifiers should assess emissions from unusual circumstances such as start up, shut down, emergency or new procedures outside the normal operating range of the facility or GHG project.

### Crosschecking GHG Information

In many cases the quantification of GHGs may be done in more than one way and/or there may be other sources of raw data. These may be used to ‘cross-check’ GHG quantifications to provide greater assurance that the reported information is within the expected range.

Types of crosschecks may include:

a) Internal checks within a process;

b) Internal checks within an organization;

c) Checks within a sector;

d) Checks against international information.

### Example—Crosschecking GHG Information: A Coal-Fired Electricity Generator

A generation company owns 3 plants at Sites A, B and C.

As part of plant operational control at Site A, the mass of coal injected is measured continually; the carbon and energy content of the coal is sampled regularly; and the fly ash mass and deposited carbon is measured regularly. From this information and stoichiometric mass balance equations, the mass of CO2 emitted can be calculated.

Crosscheck 1: The generator measures MegaWatts (MWh) of electricity produced as part of operational control, and from previous data (eg, last year's accounts) the company will have an estimate of tCO2/MWh produced. This is checked against current intensity, and any significant departures investigated. Further, manufacturers specifications state expected outputs under known maintenance conditions, and this can be used as a 2nd internal check, with significant departures investigated.

Crosscheck 2: At Site B, the company has compiled similar information, and can check whether Site A and Site B emissions are comparable. Site B may be a different plant design, and/or use a different feedstock, but the company will know that Site B is typically 4% more emission intensive than Site A. Any significant departures from this difference in current calculations can be investigated for Site A and Site B.

Crosscheck 3: The company operates within a national grid, and the national grid operating authority produces annual intensity figures for each region within the grid. The company can check whether Sites A, B and C are close to their regional average, and any significant departures investigated or explained.

Crosscheck 4: International bodies such as the IPCC produce typical emission intensity figures for known technologies. These can be used to check the approximate magnitude of the calculated emissions for Sites A, B and C, and any significant departures explained or investigated.

NOTE—None of these crosschecks on their own are a substitute for source data, but they are all useful in detecting gross errors, and highlighting any areas in the quantification procedures, which may be unusual or may introduce higher risk. Having these crosschecks provides greater assurance.

### Guidance on Assessment of the GHG Assertion Qualifying the Validation or Verification Statement

Although circumstances that require the validator or verifier to qualify the validation or verification statement vary considerably, they can be categorized in two groups:

a) The GHG assertion(s) is affected by a departure from the requirements specified by the GHG scheme, including:

i) An inappropriate treatment (eg, incorrect global warming potentials applied during the reporting period);

ii) An inappropriate estimation or quantification of a GHG source or sink in the GHG assertion(s) (eg, overestimation of carbon stocks);

iii) A failure to disclose essential information or to present information in an appropriate manner (eg, inadequate explanation of the permanence of a carbon sink).

b) The validator or verifier is unable to obtain sufficient appropriate evidence to determine whether there has been a departure from the requirements specified by the GHG scheme. These are circumstances where the validator or verifier has not been able to apply all the tests and procedures considered necessary in the circumstances. The result is that there is not sufficient appropriate evidence to form an opinion as to whether the GHG assertion(s) is presented fairly in accordance with requirements of the GHG scheme. Such limitations may arise in a number of situations, including:

i) Circumstances related to the timing of the validator's or verifier's work (eg, a verification conducted during unplanned maintenance leading to an inability to observe operational practices or monitoring equipment in operation);

ii) Circumstances beyond the control of the organization or GHG project, or the validator or verifier (eg, destruction of GHG information in a fire);

iii) A limitation imposed or created by the organization or GHG project (eg, failure to maintain adequate GHG records).

When there is a departure from the requirements of the GHG scheme or a scope limitation, the validator or verifier must decide what type of qualification or modification to the validation or verification statement is appropriate. In addition to materiality, the validator or verifier should consider:

a) The degree to which the matter impairs the usefulness of the GHG assertion(s);

b) The extent to which the effects of the matter on the GHG assertion(s) can be determined;

c) Whether the GHG assertion(s) is, or may be, misleading even when read in conjunction with the validator's or verifier's statement.

A qualified validation or verification statement, when read in conjunction with the GHG assertion(s), normally will serve adequately to inform the intended user of any deficiencies or possible deficiencies in the GHG assertion(s).

When the validator or verifier concludes that a qualification is necessary, the validation or verification statement should clearly draw attention to the qualification by modifying the validation or verification statement. The statement should include:

a) A qualification paragraph, inserted between the scope and opinion paragraphs of the statement, that includes:

i. All qualifications;

ii. An adequate explanation of the reasons for each qualification;

iii. A clear indication of how and, when reasonably determinable, to what extent the GHG assertion(s) are or may be affected;

iv. If the affect on the GHG assertion(s) of the matter causing the qualification is not reasonably determinable, a statement of such and reasons for the determination.

b) The opinion paragraph should include:

i) Wording appropriate for the type of qualification(s);

ii) A reference to the qualification paragraph.

In addition, when the qualification results from a limitation in the scope, the scope paragraph should contain a reference to the qualification paragraph.

### Adverse Validation or Verification Statements

When, in the judgment of the validator or verifier, a qualification is not appropriate, an adverse validation or verification statement can be issued (eg, the GHG assertion(s) is not presented fairly in accordance with GHG scheme requirements) or the validator or verifier can issue a statement that states the validator or verifier was unable to obtain sufficient appropriate objective evidence to form an opinion as to whether the GHG assertion(s) are presented fairly in accordance with the GHG scheme requirements.

### Guidance on the Completion of Validation and Verification Working Papers, Audit Trails and Document Control and Management

The validation or verification team should document matters that are important in providing evidence to support the validation or verification statement and evidence that the validation or verification was carried out in accordance with the agreed scope and objectives of the validation or verification and any relevant principles or requirements of GHG schemes or standards.

The validation or verification team should prepare documentation that is sufficiently complete and detailed to provide an overall understanding of the process. As appropriate, the validation or verification team should consider producing and recording the following kinds of documents and validation or verification evidence.

### Background

a) The organization or project's GHG assertion(s);

b) Information concerning the industry, GHG reporting environment and legislative environment within which the organization or GHG project operates;

c) Information concerning organizational boundaries of the organization or GHG project;

d) Information on the identification and selection of GHG sources and sinks;

e) Procedures for quantifying of GHG emissions, removals, emission reductions or removal enhancements;

g) An annotated process flow diagram, characterizing mass or energy flows for selected GHG sources and sinks;

f) A mass balance, energy balance and/or other quantitative balance for selected GHG sources and sinks;

g) Extracts or copies of important agreements, contracts and, where applicable, emissions trading and carbon offset records.

### Validation or Verification Process

a) Evidence of the planning process, including details of the anticipated and actual objectives, scope, criteria and activities to be undertaken within the validation or verification programme;

b) Details of the GHG information sampling plan, including explanations and justifications for the approach taken during the validation or verification and the methodologies used;

c) Details of the reported GHG information that was validated or verified, including any relevant supporting information that may be required to verify consistency in future validations or verifications;

d) Evidence that the validation or verification team has a clear understanding of the organization or GHG project's GHG information management and internal control systems;

e) Records relating to validation or verification team personnel, including validator/verifier competence and performance evaluation, team selection and maintenance and improvement of competence;

f) Results of the strategic review, risk assessment and materiality analysis;

g) Analyses of significant ratios and trends in the GHG information and, including those that may influence changes in the level of GHG performance;

h) Evidence of inherent and control risk assessments;

i) Analyses of GHG information inputs, quantification and aggregation and disaggregation methodologies;

j) A record of the nature, timing and extent of activities performed (including the use of any experts) and the results of such activities, including the analytical testing undertaken and significant validation and verification trails followed and the reasoning behind them;

k) A record of who completed the activities, when they were performed and how these activities contributed to the validation or verification findings and conclusions;

l) The validation or verification team's reasoning and rationale on all significant matters that require the exercise of professional judgement;

m) Any changes made to the validation or verification plan and associated activities and analytical testing as a result of evidence obtained;

n) The results and findings of the validation or verification;

o) Conclusions reached by the validation or verification team concerning significant 2081 aspects of the validation or verification, including how exceptions and unusual 2082 matters, if any, were resolved or treated. If the client made changes to original GHG assertion(s) and GHG information in order to reduce or remove the risk of material misstatement within their GHG information, reasons should be recorded.

### Communication and Reporting

a) Copies of written communications with the client, experts and other stakeholders;

b) Notes of significant verbal communications with the client, experts and other stakeholders;

c) Copies of notes of significant verbal communications and written communications with all parties involved in the validation or verification, including the terms of the validation or verification and material weaknesses in internal control;

d) Any non-conformities raised and their associated preventive and corrective action programmes, including situations where omissions or errors are considered material, resulting in amendments to the original GHG information;

e) Validation or verification follow-up reports (if applicable);

f) Copies of the responsible party's GHG assertion(s) reported to the GHG scheme and the validation or verification report and statement (where appropriate);

g) Confidentiality, safe custody, retention and ownership of validation or verification documentation.

The validation or verification body should adopt appropriate procedures for maintaining the confidentiality and safe custody of the validation or verification documentation and for retaining them for a period sufficient to meet the needs of the client, the responsible parties, the GHG scheme(s) to which they subscribe and in accordance with legal and professional requirements of record retention.

Validation or verification documentation remains the property of the validation or verification body. Although portions of, or extracts from, the validation or verification documentation may be made available to the client and/or organization or GHG project (or, where specific disclosure requirements exists, any GHG schemes to which they subscribe), at the discretion of the validation or verification body. Such disclosed documentation should not be considered as a substitute for the organization or GHG project's GHG records.

NOTE—The release of any information should be agreed with the client and/or the responsible party depending on the scope and objectives of the validation or verification and the GHG scheme rules under which the validation or verification is taking place.

### Guidance on the Validation Report

To be completed

### Guidance on the Validation and Verification Statement

The validation or verification statement should include the following elements:

a) Title;

b) Name, address and other relevant contact information for the responsible party and/or the client;

c) Opening or introductory paragraph containing:

i) Identification of the organization or GHG project's GHG assertion(s) against which the validation or verification testing was conducted;

ii) A statement of the roles and responsibilities of the organization or GHG project's management and the roles and responsibilities of the verification or validation body.

d) A scope paragraph containing:

i) Reference to the principles and requirements of relevant GHG schemes or standards against which the validation or verification was conducted;

ii) Reference to the validation or verification scope, objectives and criteria agreed with the client, including the level of assurance required;

iii) A description of the work the validation or verification team performed, including the techniques and processes used to test the GHG information and associated GHG assertion.

e) Opinion paragraph containing:

i. A reference to the GHG reporting framework and/or GHG scheme requirements (as appropriate) used to prepare the GHG assertion(s);

ii. GHG information or performance validated or verified (eg, GHG emissions, removal, emission reductions, removal enhancements);

iii. The level of assurance provided by the validation or verification, in line with the agreed validation or verification scope, objectives and criteria;

iv. An expression of opinion on the GHG assertion(s), including any limitations or qualifications to the opinion (eg, as a result of a GHG project that is still at the planning stage).

f) The date of the validation or verification statement;

g) The validation or verification body's contact details;

h) An authorised signature from the validation body.

A measure of uniformity in the form and content of the validation or verification statement is desirable because it helps to promote the Router's understanding and to identify unusual circumstances when they occur.

The following additions may be added to the validation or verification statement to provide further clarity:

a) The credentials of the validation or verification body to add credibility to the assurance;

b) The degree to which the validation or verification body is independent of the client, the responsible party and the subject matter;

c) If there are reports from two or more validation or verification bodies, their respective responsibilities.

NOTE—The validation or verification body should produce a draft validation or verification statement to be sent to the client and/or the responsible party to review for factual correctness. If the responsible party is satisfied that the validation or verification statement is factually correct then the validation or verification body is able to release the validation or verification statement in final form. If the responsible party requires any significant amendments to be made to the draft statement then the revised content has to be agreed with the team leader prior to publication.

### Annex B (Informative)

### Guidance on Skills and Competencies for Validators and Verifiers

Annex B provides guidance on the skills and competencies required of validators and verifiers to effectively conduct validation and verification requirements contained in Clause 5 of this International Standard. Annex B is informative and does not include mandatory requirements.

### General Guidance on the Skills and Competencies for Validators and Verifiers

The validation or verification body should ensure that personnel involved in validation or verification work be competent for the functions they perform. In the validation or verification of GHG information, the personnel involved are likely to include those who:

a) Manage the validation or verification process;

b) Assess new and continuing clients, including making the decision to accept or decline the client;

c) Select and verify the competence of validators or verifiers to conduct the validation or verification;

d) Brief team members and arrange any necessary training;

e) Assess applications from clients and conduct the strategic review;

f) Undertake the validation or verification activities;

g) Review validation or verification reports, working papers and associated supported evidence from the validation or verification process;

h) Make the decisions on validation or verification and the validation or verification statement;

i) Manage the storage of records and information;

j) Set-up and operate a procedure for complaints, disputes and appeals.

### Personal Attributes of Validators and Verifiers

Validation or verification team members should possess personal attributes to enable them to act in accordance with the principles of validation or verification described in Clause 4.

A validation or verification team member should be:

a) Ethical (ie, fair, truthful, sincere, honest and discreet);

b) Open-minded (ie, willing to consider alternative ideas or points of view);

c) Diplomatic (ie, tactful in dealing with people);

d) Observant (ie, actively aware of physical surroundings and activities);

e) Perceptive (ie, instinctively aware of and able to understand situations);

f) Versatile (ie, adjusts readily to different situations);

g) Tenacious (ie, persistent, focused on achieving objectives);

h) Decisive (ie, reaches timely conclusions based on logical reasoning and analysis);

i) Self-reliant (ie, acts and functions independently while interacting effectively with others).

### Composite Knowledge and Skills Requirements for the Validation or Verification Team

The validation or verification team should consist of one or more team leaders, together with an appropriate combination of validators or verifiers and/or independent experts as appropriate to the agreed scope of the validation or verification.

All validation or verification team members involved in the validation or verification should be familiar with:

a) The subject matter relating to the scope of the validation or verification;

b) The legal rules under which the validation or verification is being undertaken (eg, the parameters of any legal documents or contracts agreed between the GHG scheme administrators and the responsible party);

c) Any specific principles or requirements of the GHG scheme or standard that fall within the scope of the validation or verification;

d) Any accreditation requirements incumbent on the validation or verification body conducting the work;

e) The industrial processes that generate GHG emissions and the technical issues associated with their quantification, monitoring and reporting;

f) The biological systems that affect GHGs removals and the technical issues associated with their quantification, monitoring and reporting;

g) GHG emission or emission reduction quantification, monitoring and reporting methodologies used by the organization or GHG project;

h) GHG removal or removal enhancement quantification, monitoring and reporting methodologies used by the organization or GHG project;

i) Data and information auditing and sampling methodologies;

j) Risk assessment methodologies and materiality analysis approaches;

k) The validation or verification body's procedures (administrative and otherwise) for the performance of the validation or verification work.

At least one validation or verification team member should have detailed knowledge of each of the above areas based on relevant working experience.

In addition to the above, the validation or verification team should collectively have experience, training and up-to-date knowledge of:

a) The activities required to identify failures in GHG reporting systems and decide on its impact on the organization or GHG project's GHG assertion(s);

b) The sources and types of GHG sources or sinks selected by the organization or GHG project;

c) The GHG quantification methodologies to be used by the organization or GHG project;

d) Other competencies specific to the GHG scheme (eg, political and legal expertise for GHG projects under the Kyoto Protocol);

e) Current best practice in the field.

The validation or verification team composition and competence should also take account of the scope of the responsible party's GHG programmes or GHG projects and the nature of the GHG reporting system. Key considerations should include:

a) Whether the scope is restricted to CO2 or includes other GHGs;

b) The complexity of the GHG information under consideration (ie, is it based solely on fuel/energy use metered by electricity/fuel bills or are emissions largely process-based?);

c) The nature of the computer information system used to collect and report GHG information (ie, is it a complex database system requiring working knowledge of information technology systems or is it a simple spreadsheet system?);

d) The complexity surrounding the organization or GHG project's operations (ie, is the validation or verification activity to be conducted at a single site or over multiple sites that involve careful consideration of joint venture or outsourcing arrangements?);

e) The complexity of the GHG project (ie, is the project a simple energy swap scenario or the complicated, detailed assessment of a new and novel technology?);

f) The prevailing legal and regulatory framework within which the organization or GHG project is operating (ie, are the organization or GHG project's activities heavily regulated under national, regional or international law or is the legal and regulatory situation much more simplistic?).

### Specific Knowledge and Skills Requirements for Validation or Verification Team Leaders and Peer Reviewers

Validation or verification bodies should ensure that team leaders and peer reviewers have the appropriate skills and competencies to fulfil the following key responsibilities:

a) Checking that the validation or verification team meets the necessary competency requirements;

b) Leading the team and managing the validation or verification process;

c) Understanding the agreed scope of the validation or verification and its relationship to a GHG scheme(s) (if appropriate);

d) Ensuring that the validation or verification objectives are addressed in the validation or verification planning;

e) Resolving issues relating to validation or verification, in particular those associated with materiality issues and shifts in the risk profile of the reported GHG information;

f) Directing the drafting of the verification report and statement and communicating or distributing them to the peer reviewer;

g) Ensuring all validation or verification documentation, including working papers, supporting evidence, recommendations and the draft report and statement are complete;

h) Providing assistance to the peer reviewer in order to complete the validation or verification.

### Levels of Education, Work Experience, Training and Experience for Those Conducting Validations or Verifications

Validation or verification team members should have the following education, work experience, training and experience:

a) Completed an education sufficient to acquire the knowledge and skills described above;

b) Have work experience that contributes to the knowledge and skills described above. At least some of this work experience should be in a technical, managerial or professional position involving the exercise of judgement, problem solving, and communication with other managerial or professional personnel, peers, clients and other stakeholders;

c) Have received formal training and gained practical experience related to the activities described in Clause 5 of this International Standard and the associated guidance notes in Annex A, preferably under the direction and supervision of a validation or verification team leader.

d) Validation or verification team leaders and peer reviewers should have acquired additional experience to develop the knowledge and skills described above.

### Maintenance and Improvement of Competence

Continual professional development is concerned with the maintenance and improvement of knowledge, skills and personal attributes. This can be achieved through additional work experience, training, private study, coaching, attendance at meetings, seminars and conferences or other relevant activities. Validation or verification team members should demonstrate their continual professional development.

The continual professional development activities should take into account changes in the needs of the individual and the organization, the practice of GHG information validation or verification and principles and requirement of GHG schemes and standards.

Validation or verification team members should maintain and demonstrate their GHG information validation or verification abilities through regular participation in GHG validations and/or verifications.

### Validator or Verifier Evaluation

The evaluation of validation or verification team members and team leaders should be planned, implemented and recorded in accordance with the validation or verification body's procedures to provide an outcome that is objective, consistent, fair and reliable. The evaluation process should identify training and other skill enhancement needs.

The evaluation of validators and verifiers should occur at the following stages:

a) The initial evaluation of persons who wish to become validation or verification team members;

b) The evaluation of persons as part of the validation or verification team selection process described in Clause 5.2.1;

c) The continual evaluation of validation or verification team member's performance to identify needs for maintenance and improvement of knowledge and skills.

### Summary of the American Carbon Registry Standard v5.0

For purposes of this discussion, the American Carbon Registry standard will be used as the model for carbon credit registry design, offset and credit capture, storage, and trading. The standard is included below:

The American Carbon Registry® (ACR) is a leading carbon offset program with two decades of unparalleled carbon market experience in the development of rigorous, science-based offset standards and methodologies as well as operational experience in the oversight of offset project verification, registration, offset issuance and retirement reporting through

ACR's online registry system. ACR is a non-profit enterprise of Winrock International. Winrock International works with people in the U.S. and around the world to empower the disadvantaged, increase economic opportunity, and sustain natural resources. Key to this mission is building capacity for climate change mitigation and adaptation and leveraging the power of environmental markets. Since the 1990s, Winrock has been a leader in developing science-based GHG measurement and monitoring methods and protocols.

ACR was founded in 1996 as the GHG Registry by the Environmental Resources Trust, and joined Winrock International in 2007. As the first private greenhouse gas registry in the world, ACR has set the bar for offset quality that is the market standard today and continues to lead carbon market innovation.

In 2012 ACR was approved by the California Air Resources Board to serve as an Offset Project Registry (OPR) and Early Action Offset Program (EAOP) for the California cap-and-trade market. ACR's work as a California OPR is governed by the California cap-and-trade regulation and compliance offset protocols approved by the Air Resources Board. 1 The ACR Standard governs only the registration of projects registered under ACR-approved methodologies.

### ACR Governance

The ACR program is built on principles of accountability, transparency, responsiveness, and participatory processes. As an enterprise of Winrock International, ACR benefits by the support and guidance of an established, reputable, global nonprofit organization. Winrock International's management, executive team, and board of directors provide direct oversight of all ACR operations.

### The ACR Standard

The ACR Standard details ACR's requirements and specifications for the quantification, monitoring, and reporting of project-based GHG emissions reductions and removals, verification, project registration, and issuance of offsets. The Standard establishes the quality level that every project must meet in order for ACR to register its GHG emissions reductions and removals as tradable environmental assets.

ACR aims to maximize flexibility and usability for Project Proponents, while maintaining the environmental integrity and scientific rigor necessary to ensure that projects developed against its standards and methodologies are recognized as being of the highest quality, whether used for voluntary or pre-compliance early action purposes.

Adherence to this Standard, relevant sector-specific standards, and associated methodologies will ensure that project-based offsets represent emissions reductions and removals that are real, measurable, permanent, in excess of regulatory requirements and common practice, additional to business-as-usual, net of leakage, verified by a competent independent third party, and used only once.

### Applicability

Project Proponents wishing to develop a project for registration on ACR shall follow this Standard and any relevant ACR sector standard, and must apply an ACR-approved methodology (as defined below).

The ACR Standard v5.0 supersedes the ACR Standard v4.0 (January 2015). Any project listed or registered subsequent to Jul. 1, 2017 must follow all requirements of the ACR Standard v5.0. Projects currently listed or registered, or listed or registered prior to Jul. 1, 2017, may be validated and verified according to ACR Standard v4.0 through the end of the Crediting Period.

Project Proponents and other interested parties should refer to www.americancarbonregistry.org for the latest version of the ACR Standard, sector standards, methodologies, tools, document templates, and other guidance.

### Chapter Guide

Chapter 1 provides basics on ACR

Chapter 2 provides ACR's general accounting and data quality principles for offset projects.

Chapter 3 summarizes ACR project eligibility requirements.

Chapter 4 details the ACR tests to ensure that offset projects are additional to business-as-usual.

Chapter 5 describes ACR's approach to ensuring permanence of GHG reductions and removals.

Chapter 6 summarizes the process for Project Proponents to develop and register a project

Chapter 7 summarizes the processes for ACR approval of new methodologies and methodology modifications

Chapter 8 summarizes ACR requirements for ensuring Environmental and Community Safeguards

Chapter 9 summarizes ACR requirements for validation and verification of all projects by a competent independent third-party verifier, which are addressed in greater detail in the ACR Validation and Verification Standard for GHG Projects.

Chapter 10 addresses ACR linkages to other GHG Programs and Registries, Emission Trading Systems and National or Sectoral GHG Emissions Reduction Targets

Appendix A provides definitions of terms used throughout this document. Appendix B provides a list of normative references on which the ACR Standard is based. Appendix C is ACR's Appeals and Complaints Procedure.

The ACR Standard does not detail legal responsibilities of ACR and ACR members with regard to the use of the registry, which are provided for in the ACR Member Terms of Use Agreement and referenced operative documents such as the ACR Operating Procedures. A project-specific contract between ACR and Project Proponents governs the operation of a buffer account to mitigate the risk of reversals in certain types of projects.

### Citation

The appropriate citation for this document is: American Carbon Registry (2017). The American Carbon Registry Standard, version 5.0. Winrock International, Little Rock, Ark.

### Chapter 1: ACR Basics

### A. Description of the ACR

The American Carbon Registry®, a nonprofit enterprise of Winrock International, is a leading carbon offset program that operates in both the voluntary and the regulated carbon markets. Founded in 1996 as the first private voluntary GHG registry in the world, ACR has two decades of unparalleled carbon market experience in the development of rigorous, science-based offset standards and methodologies as well as operational experience in the oversight of offset project verification, registration, offset issuance and retirement reporting.

ACR operates a transparent online registry system for members to register projects and record the issuance, transfer and retirement of serialized, project-based and independently verified offsets. ACR's registry system records transactions directly negotiated between buyer and seller and is not an exchange. Offset transactions take place outside of ACR, over-the-counter (OTC) or on exchanges, and are tracked on ACR through the unique serial numbers assigned to every offset.

### B. Objectives

ACR's objectives are to:

□ Encourage action to manage GHG emissions; □ Provide guidance, transparent infrastructure, and science-based standards to foster high quality reductions in GHG emissions; □ Support best practices in project-level GHG accounting; □ Commercialize innovative new methodologies; □ Encourage broad adoption of climate change-mitigating practices with significant community, economic and environmental benefits; □ Enhance public confidence in market-based action for GHG reduction; □ Support convergence of international and U.S. carbon markets.

### C. Geographic Scope

ACR accepts projects from locations worldwide, provided they follow an ACR-approved methodology. Some methodologies prescribe a narrower geographic scope (e.g., United States only).

### D. Scope: Greenhouse Gases and Particulate Matter

ACR registers emission reductions and/or removal enhancements of carbon dioxide (CO2), methane (CH4), nitrous oxide (N2O), hydrofluorocarbons (HFCs), perfluorocarbons (PFCs), sulfur hexafluoride (SF6) and black carbon. ACR's scope also includes destruction of Ozone-Depleting Substances (ODS) listed in Annexes A, B, C and E of the Montreal Protocol.2

### E. Scope: Project Types

ACR accepts all projects validated and verified against an ACR-approved methodology, provided they comply with the current versions of the ACR Standard and relevant sector standard if applicable. ACR approved methodologies include: □ Methodologies developed by ACR and approved through the public consultation and scientific peer review process; □ Methodologies approved by the Clean Development Mechanism (CDM) Executive Board, provided that the project is implemented in a non-Annex I country and adheres to requirements of the ACR Standard; □ Methodologies approved by the CDM Executive Board, provided that if the project will be implemented in the United States or another Annex I country, the Project Proponent must first have ACR review and approve the CDM methodology for consistency with ACR requirements; □ Modifications of existing methodologies, provided such modifications have been approved by ACR per requirements found in Chapter 7; □ New methodologies developed by external authors and approved by ACR through ACR's methodology development process described in Chapter 7.

With the exception of hydropower, ACR accepts renewable energy projects 100 MW and under and energy efficiency projects where the baselines include indirect emissions, only if the project activity takes place in the developing world.3 For hydropower, ACR accepts run-of-river projects up to 10 MW.

ACR will register GHG reductions from renewable energy and energy efficiency projects in the United States only if all of the following criteria are met: □ The project displaces direct emissions by reducing the consumption of fossil fuels at a facility that the Project Proponent owns or controls, or for which the facility owner has assigned the Project Proponent clear and uncontested offsets title. Examples are biomass co-firing with coal, biogas used to displace natural gas, energy efficiency projects that reduce natural gas use, etc.;

2 See http://ozone.unep.org/Publications/MP_Handbook. 3 Under the Kyoto Protocol's Clean Development Mechanism (CDM), the governments of developing countries (non-Annex 1 countries), by approving emission reduction projects from renewable energy projects, provide a de facto assignment of emission reduction property rights to Project Proponents instead of owners of fossil fuel power plants. By contrast, renewable energy Project Proponents in Annex 1 countries (industrialized countries) do not have an assignment of emissions reduction property rights by the government, and thus do not have an unambiguous and uncontested ownership claim to the emission reductions.

□ The project meets additionality and other requirements of the ACR Standard; □ The GHG reductions have not been used to meet a regulatory compliance obligation under a binding limit; □ Under possible future U.S. climate regulations, the project does not take place at a regulated source; □ The project has not been counted toward a mandatory Renewable Portfolio Standard (RPS) obligation or claimed Renewable Energy Credits (RECs), unless regulations in the relevant jurisdiction clearly allow separation (“unbundling”) of RECs and GHG attributes or the sources of REC and GHG attributes are clearly distinct.

### ACR's Scope Excludes:

□ Projects that do not meet all ACR eligibility criteria, including projects which convert and/or clear native ecosystems to generate carbon offsets; □ Renewable energy and energy efficiency projects in the U.S., unless meeting all criteria above. Projects that displace indirect emissions at a source not owned or controlled by the Proponent (e.g., grid-connected renewable power generation) do not meet these criteria because of the lack of unambiguous and uncontested ownership of the emission reductions, lack of clear additionality, potential for double-counting of offsets and RECs in markets where regulations do not clearly allow for unbundling of RECs and GHG attributes, or where the sources of such attributes are indistinct, and potential for double-counting of offsets and entity-level emissions reductions; □ Energy or life-cycle GHG accounting-based indirect emissions reductions and removals from projects originating in Annex I countries.

### F. Language

The operating language of ACR is English. All GHG Project Plans, methodologies, tools, verification statements, and other documents required by ACR shall be in English.

### G. Unit of Measure

Project Proponents shall calculate, quantify, and report all GHG reductions and removal enhancements in metric tons, converting each metric ton to its CO2 equivalent (CO2e) using calculations based on the 100-year Global Warming Potential (GWP) factors listed in the IPCC Fourth Assessment Report (AR4), Working Group 1, Chapter 2, Table 2.14.4

### H. Unit of Exchange

The ACR unit of exchange is a verified emissions reduction (VER), serialized and registered as an Emission Reduction Ton (ERT), denominated in metric tonnes of CO2e. ERTs include both emission reductions and removal enhancements (i.e. enhanced sequestration).

### I. No Ex-Ante Crediting

A project-based offset is the result of a defined and eligible project action that yields quantifiable and verifiable GHG emissions reductions/removals. ACR will not issue ERTs for GHG emissions reductions or removals when an emission mitigation activity has not occurred or is not yet verified. ACR will not credit a projected stream of offsets on an ex-ante basis.

### J. Adoption of and Revisions to ACR Standards

All ACR standards, including the ACR Standard and sector-specific standards, will be posted for public comment for at least 30 days before adoption. ACR will prepare responses to all submitted comments and post the comments and responses along with the new version of the standard.

The ACR Standard and any sector specific standards shall be reviewed and revised, as necessary, by ACR, at minimum, every three years.

Such updates occur when significant changes to GHG accounting best practice or the legislative and/or regulatory context justify an update; when new provisions or requirements originating in methodologies make ACR aware of higher-level requirements or clarifications that should be made at the ACR Standard or sector standard level; or for other reasons.

On a project level and in certain circumstances, ACR may require all projects, including those validated under a previous version of the ACR Standard, to immediately implement a policy or process revision, such as updated administrative and reporting procedures, detailed in a subsequent version of the ACR Standard.

### K. Conflict of Interest Policy

As a non-profit organization that values its reputation for integrity, Winrock International requires that all management and staff adhere to its Code of Professional Conduct, which includes a strict and comprehensive policy against engaging in activities that present a conflict of interest. Accordingly, each director, officer, and staff member of Winrock, including ACR staff, are required to regularly affirm that they are in compliance with this policy, that they avoid all conflicts of interest and take reasonable action to avoid circumstances that create the appearance of a conflict of interest. Winrock and ACR staff are required to notify management immediately if any conflict of interest situations arise or come to their attention so that the conflict can be appropriately mitigated.

In addition to its internal conflict of interest policy, ACR also requires that its third party registry service provider maintain and adhere to a strict conflict of interest policy and that all ACR-approved Validation and Verification Bodies (VVBs) execute an Attestation of Validation/Verification Body, which defines the VVB role and responsibilities and ensures technical capabilities of all staff and no conflicts of interest. ACR Approved VVBs must also execute a project-specific conflict of interest form for each project validated and/or verified, which is reviewed and approved by ACR.

### Chapter 2: Accounting and Data Quality Principle

The accounting and data quality principles summarized here are designed to ensure that the assumptions, values, and procedures used by Project Proponents and Validation/Verification Bodies (VVBs) result in a fair and true accounting of GHG emission reductions and removals.

### A. Guiding Principles for GHG Accounting

ACR affirms a set of guiding principles, based on the ISO 14064 Part 2 (2006) specifications. These are summarized in Table 1.

### Table 1—Core GHG Accounting Principles

### Relevance

Select the GHG sources, GHG sinks, GHG reservoirs, data and methodologies appropriate to the needs of the intended user (ISO 140642:2006, clause 5.6).

### Completeness

Include all relevant GHG emissions and removals. Include all relevant information to support criteria and procedures (ISO 14064-2:2006, clause 5.3).

### Consistency

Enable meaningful comparisons in GHG-related information. Use consistent methodologies to allow for meaningful comparisons of emissions over time. Transparently document any changes to the data, boundary, methods, or any other relevant factors.

### Accuracy

Reduce bias and uncertainties as far as is practical. Ensure that the quantification of GHG emissions is systematically neither over nor under actual emissions, as far as can be judged, and that uncertainties are reduced as far as practicable. Achieve sufficient accuracy to enable users to make decisions with confidence as to the integrity of the reported information (WRI/WBCSD, Corporate Inventory Guidance, 2007).

### Transparency

Disclose sufficient and appropriate GHG-related information to allow intended users to make decisions with reasonable confidence. Address all relevant issues in a factual and coherent manner, based on a clear audit trail. Disclose any relevant assumptions and make appropriate references to the accounting and calculation methodologies and data sources used. (WRI/WBCSD, Corporate Inventory Guidance, 2007).

### Conservativeness

Use conservative assumptions, values and procedures to ensure that GHG emission reductions or removal enhancements are not overestimated (ISO 14064-2:2006, clause 3.7).

### B. Boundary Selection

GHG project boundaries include a project's physical boundary or implementation area, the GHG sources, sinks and reservoirs (or pools) considered, and the project duration.

Approved methodologies establish criteria for the selection of relevant GHG sources, sinks and reservoirs for regular monitoring or estimation. The Project Proponent shall justify in the GHG Project Plan the exclusion from regular monitoring of any relevant GHG source, sink or reservoir.

In accordance with ISO 14064-2:2006, approved methodologies establish criteria and procedures for quantifying GHG emissions and/or removals for selected GHG sources, sinks and/or reservoirs. The Project Proponent shall quantify GHG emissions and/or removals separately for each relevant GHG for each GHG source, sink and/or reservoir identified in the methodology as being relevant for the project and for the baseline scenario.

The Project Proponent shall provide a detailed description of the geographic boundary of project activities. The project activity may contain more than one facility or discrete area of land, but each facility or land area must have a unique geographical identification, and each land area must meet the land eligibility requirements of the relevant ACR sector standard, if applicable. For Agriculture, Forestry and Other Land Use (AFOLU) projects, the Project Proponent shall provide maps, Geographic Information System (GIS) shapefiles, or other relevant information to delineate the project boundary.

ACR sector standards specify the required Minimum Project Term for particular project types.

### C. Relevance and Completeness

Consistent with ISO 14064 Part 2, Project Proponents shall consider all relevant information that may affect the accounting and quantification of GHG reductions and removals, including estimating and accounting for any decreases in carbon pools and/or increases in GHG emission sources.

### D. Uncertainty, Accuracy and Precision

The Project Proponent shall reduce, as far as is practical, uncertainties related to the quantification of GHG emission reductions or removal enhancements.

For methodologies based on statistical sampling (for instance, methodologies in the forestry or land use sectors often employ statistical sampling requirements), ACR requires that in order to be allowed to report the mean of the estimated emission reduction/removal, the 90% statistical confidence interval of sampling must be no more than 10% of the mean. If the Project Proponent cannot meet the targeted ±10% of the mean at 90% confidence, then an uncertainty deduction is required. Project-specific methodologies provide guidance how to calculate this uncertainty deduction. Methodologies submitted for ACR approval shall include methods for estimating uncertainty relevant to the project and baseline scenario.

ACR leaves to the Project Proponent the decision whether the potential additional revenues from reporting the mean without an uncertainty deduction justify the additional costs of more intensive sampling to achieve precision of +10% of the mean at 90% confidence.

The use of biogeochemical or process models must also include an estimate of structural uncertainty related to the inadequacy of the model, model bias, and model discrepancy. This should be quantified using the best available science, and can include Monte Carlo analyses, uncertainty estimates from peer reviewed literature, and/or consulting model experts who have either developed or worked directly with the model in an academic setting.

### E. Conservativeness

The Project Proponent shall select assumptions and values to ensure that GHG emission reductions and removals are not overestimated, particularly in the event that the Proponent relies on uncertain data and information. For GHG sources, sinks and reservoirs not selected for regular monitoring, the Project Proponent shall estimate GHG emissions and/or removals by the sources, sinks and reservoirs relevant for the project and those relevant for the baseline scenario. When reporting emissions data to ACR for offset issuance the following rules shall be applied:

a. Claimed emissions reductions shall be rounded down to the nearest whole number; b. Calculated buffer pool contributions shall be rounded up to the nearest whole number.

### F. Emissions Factors

Where needed to estimate GHG emission reductions or removal enhancements in the project or baseline scenario, the Project Proponent shall select or develop GHG emissions or removal factors that:

□ Derive from a scientific peer-reviewed origin; □ Are appropriate for the GHG source or sink concerned; and □ Take account of the quantification uncertainty.

### G. Managing Data Quality

The Project Proponent shall establish and apply quality assurance and quality control (QA/QC) procedures to manage data and information, including the assessment of uncertainty in the project and baseline scenarios. QA/QC procedures shall be outlined in the GHG Project Plan.

### Chapter 3: Project Eligibility Requirements

Table 2 details ACR eligibility criteria for all projects, provides a definition of each criterion, and articulates ACR requirements. Eligibility requirements for specific project types are summarized in the relevant ACR sector standard and/or methodology. Project Proponents shall address, in their GHG Project Plan, each of the criteria below.

### Table 2—Eligibility Requirements for Offset Projects

### Criterion Definition ACR Requirement

Start Date5,6 ACR defines the Start Date for all projects other than AFOLU as the date on which the project began to reduce GHG emissions against its baseline.

ACR defines the Start Date for AFOLU projects as the date on which the Project Proponent began the activity on project lands, with more specific guidance in the relevant ACR sector standard and methodology.

Non-AFOLU Projects—Approved Methodology:

Projects must be validated within two years of the project start date.

Non-AFOLU Projects—Newly Approved Methodology7:

Projects using a new methodology must be validated within three years of the project start date.

AFOLU Projects—Approved Methodology:

Projects must be validated within three years of the project start date.

AFOLU Projects—Newly Approved Methodology:

Projects using a new methodology must be validated within four years of the project start date.

5 The start date requirements do not apply to existing ACR projects that renew a crediting period. In these instances, the initial project start date, as previously validated, shall apply and shall be accepted in the crediting period renewal validation process on a de facto basis. 6 Projects transferring to ACR from another GHG program and that have reached the end of a crediting period, may apply for an initial crediting period at ACR per ACR Standard requirements. The project must have been successfully validated and/or verified at the previous GHG program and must have a validated/verified start date of Jan. 1, 2000 or after. 7 A methodology is considered “Newly Approved” if ACR has approved the methodology no more than 6 months prior to the project's listing or registration with ACR.

See Chapter 6 for ACR listing and registration requirements.

### Criterion Definition ACR Requirement

### Minimum Project Term

The minimum length of time for which a Project Proponent commits to project continuance, monitoring and verification.

The Minimum Project Term for specific project types is specified in the relevant ACR sector standard and/or methodology. Project types with no risk of reversal subsequent to crediting have no required Minimum Project Term.

### Crediting Period

Crediting Period is the finite length of time for which a GHG Project Plan is valid, and during which a project can generate offsets against its baseline scenario.

Crediting Periods are limited in order to require Project Proponents to reconfirm, at intervals appropriate to the project type, that the baseline scenario remains realistic and credible, the Project Activity remains additional, and GHG accounting best practice is being used. This is important since once a project has demonstrated its additionality, it is not required to do so again until applying to renew the Crediting Period.

The Crediting Period for non-AFOLU projects shall be ten (10) years. AFOLU projects may have longer Crediting Periods, as specified in the relevant ACR sector standard or methodology.

A Project Proponent may apply to renew the Crediting Period by complying with all then-current ACR requirements, re-evaluating the baseline scenario, and using emission factors, tools and methodologies in effect at the time of Crediting Period renewal. Except where specified in a methodology, ACR does not limit the allowed number of renewals.

Projects that are deemed to meet all ACR additionality criteria are considered additional for the duration of their Crediting Period. If regulations or common practice change during the Crediting Period, this may make the project non-additional and thus ineligible for renewal, but does not affect its additionality during the current Crediting Period, unless otherwise specified in the project-specific methodology.

Real A real offset is the result of a project action that yields quantifiable and verifiable GHG emissions reductions and/or removals.

GHG reductions and removals shall result from an emission mitigation activity that has been conducted in accordance with an approved ACR methodology and is verifiable. ACR will not credit a projected stream of offsets on an ex-ante basis.

### Criterion Definition ACR Requirement

### Emission or Removal Origin

An emission or removal is direct if it originates from sources or sinks over which the Project Proponent has control.

An emission or removal is indirect if it originates at sources or sinks over which the Project Proponent does not have control.

For Projects reducing or removing direct emissions, the following requirement applies:

Project Proponent shall own, have control, or document effective control over the GHG sources/sinks from which the emissions reductions or removals originate. If the Project Proponent does not own or control the GHG sources or sinks, the Proponent shall document that effective control exists over the GHG sources and/or sinks from which the reductions/removals originate.

For Projects that reduce or remove energy-related indirect emissions, eligible projects must be located in the developing world (non-Annex I countries to the UNFCCC).

For Projects reducing or removing non-energy indirect emissions,8 the following requirement applies:

Project Proponent shall document that no other entity may claim GHG emission reductions or removals from the project activity (i.e. that no other entity may make an ownership claim to the emission reductions or removals for which credits are sought).

8 ACR will not consider projects or methodologies for indirect emissions reductions/removals based on life-cycle GHG accounting methods.

### Criterion Definition ACR Requirement

Offset Title Offset title is a legal term representing rights and interests in an offset, a future stream of offsets, or a project delivering offsets.

Project Proponent shall provide documentation and attestation of undisputed title to all offsets prior to registration, including chain of custody documentation if offsets have ever been sold in the past. Title to offsets shall be clear, unique, and uncontested.

If the Project Proponent (ACR Account Holder) does not own the lands or facilities from which the GHG reductions or removals originate, the Project Proponent shall provide documentation that the owner of those lands or facilities has transferred offset title to the Project Proponent. ACR will only issue offsets into the account of a Project Proponent with clear, unencumbered and uncontested offset title.

Additional GHG emission reductions and removal enhancements are additional if they exceed those that would have occurred in the absence of the Project Activity and under a business as-usual scenario.

Every project shall use either an ACR-approved performance standard and pass a regulatory surplus test, or pass a three-pronged test of additionality in which the project must: 1) exceed regulatory/legal requirements; 2) go beyond common practice; and 3) overcome at least one of three implementation barriers: institutional, financial or technical.

### Criterion Definition ACR Requirement

### Regulatory Compliance

Adherence to all laws, regulations, and other legally binding mandates directly related to project activities.

Projects must maintain material regulatory compliance. In order to maintain material regulatory compliance, a project must not be deemed to be out of compliance, by a regulatory body(ies), at any point during a reporting period. Projects deemed to be out of compliance with regulatory requirements are not eligible to earn ERTs during the period of non-compliance. Regulatory compliance violations related to administrative processes (for instance, missed application or reporting deadlines) shall be treated on a case by case basis and may not disqualify a project from ERT issuance. Project Proponents are required to provide a regulatory compliance attestation to a verification body at each verification. This attestation must disclose all violations or other instances of non-compliance with laws, regulations, or other legally-binding mandates directly related to project activities.

### Criterion Definition ACR Requirement

Permanent Permanence refers to the longevity of removal enhancements and the risk of reversal, i.e., the risk that atmospheric benefit will not be permanent.

Reversals may be unintentional or intentional.

For projects with a risk of reversal of GHG removal enhancements, Project Proponents shall assess and mitigate risk, and monitor, report and compensate for reversals.

Project Proponents shall assess risk using an ACR approved risk assessment tool.

Project proponents will enter into a legally-binding Reversal Risk Mitigation Agreement with Winrock/ACR that details the risk mitigation option selected and the requirements for reporting and compensating reversals.

Proponents of terrestrial sequestration projects shall mitigate reversal risk by contributing ERTs from the project itself to the ACR buffer pool; contributing ERTs of another type or vintage to the ACR buffer pool; providing evidence of sufficient insurance coverage with an ACR-approved insurance product to recover any future reversal; or using another ACR-approved risk mitigation mechanism. Proponents of geologic sequestration projects shall mitigate reversal risk by contributing ERTs from the project itself to the ACR Reserve Account; contributing ERTs of another type or vintage to the ACR Reserve Account; providing evidence of sufficient insurance coverage with an ACR approved insurance product to recover any future reversal; or using another ACR-approved risk mitigation mechanism.

All projects must adhere to ongoing monitoring requirements as detailed in relevant methodologies and reversal reporting and compensation requirements as detailed in the Reversal Risk Mitigation Agreement.

### Criterion Definition ACR Requirement

Net of Leakage is an increase in GHG emissions or decrease in sequestration outside the project boundaries that occurs because of the project action.

ACR requires Project Proponents to assess, account for, and mitigate certain types of leakage, as summarized in relevant sector standards and approved methodologies. Project Proponents must deduct leakage that reduces the GHG emissions reduction and/or removal benefit of a project in excess of any applicable threshold specified in the methodology9.

### Independently Validated and Verified

Validation is the systematic, independent and documented process for the evaluation of a GHG Project Plan against applicable requirements of the ACR Standard, sector standard and approved methodology.

Verification is the systematic, independent and documented assessment by a qualified and impartial third party of the GHG assertion for a specific reporting period.

ACR requires third-party validation and verification, by an ACR-approved Validation/Verification Body (VVB), at specified intervals in order to issue ERTs. Governing documents for validation and verification are the ACR Standard, relevant sector standard, relevant methodology, and the ACR Validation and Verification Standard Verification is required prior to issuance of ERTs.

Validation and verification may occur simultaneously, and be conducted by the same ACR-approved verifier.

ACR requires verifiers to provide a reasonable (as opposed to limited) level of assurance that the GHG assertion is without material discrepancy. ACR's materiality threshold is ±5%.

9 Note that ACR may credit a positive leakage scenario in rare instances and only when quantification mechanisms are specifically included an approved ACR methodology.

### Criterion Definition ACR Requirement

### Environmental and Community Safeguards

Projects have the potential to generate both positive and negative community and environmental impacts. Appropriate safeguard procedures can identify, evaluate and manage potential negative impacts. Positive impacts can contribute to sustainable development objectives.

ACR requires that all projects develop and disclose an impact assessment to ensure compliance with environmental and community safeguards best practices. Environmental and community impacts of projects should be net positive, and projects must “do no harm” in terms of being in violation of local, national or international laws or regulations.

Project Proponents must identify community and environmental impacts of the project. Projects may disclose positive contributions as aligned with applicable sustainable development goals. Projects must describe the safeguard measures in place to avoid, mitigate or compensate for potential negative impacts, and how such measures will be monitored, managed and enforced.

ACR does not require that a particular process or tool be used for the impact assessment as long as basic requirements defined by ACR in Section 8 are addressed. ACR projects can follow internationally recognized approaches such as The World Bank Safeguard Policies or can be combined with the Climate Community and Biodiversity Alliance (CCBA) Standard or the Social Carbon Standard for the assessment, monitoring and reporting of environmental and community impacts.

Project Proponents shall disclose in their Annual Attestations any negative environmental or community impacts or claims of negative environmental and community impacts and the appropriate mitigation measure

ACR reserves the right to refuse to register or issue credits to a project based on community or environmental impacts that have not or cannot be mitigated, or that present a significant risk of future negative environmental or community impacts.

### Chapter 4: Additionality

ACR's additionality requirements are intended to ensure that credited offsets exceed the GHG reductions and removals that would have occurred under current laws and regulations, current industry practices, and without carbon market incentives. Project Proponents must demonstrate that the GHG emission reductions and removals from an offset project are above and beyond the “business as usual” scenario. To qualify as additional, ACR requires every project:

□ Either to exceed an approved performance standard, as defined in the applicable methodology, and a regulatory additionality test; □ Or to pass a three-prong test of additionality as described below.

### A. Three-Prong Additionality Test

This approach combines three tests that help determine whether GHG emission reductions and removals from an offset project are above and beyond the “business as usual” scenario. This does not mean the project activity delivers no financial or other benefits other than GHG reduction; it simply attempts to ascertain whether GHG reduction was a driving factor.

The three-prong test requires projects to demonstrate that they exceed currently effective and enforced laws and regulations; exceed common practice in the relevant industry sector and geographic region; and face at least one of three implementation barriers—financial, technological, or institutional. The three prong test is described in Table 3. The GHG Project Plan must present a credible demonstration, acceptable to ACR and the VVB, that the project passes these tests.

Some ACR-approved methodologies require application of an additionality tool to assist Project Proponents in demonstrating additionality. ACR does not require all methodologies to mandate application of an additionality tool, but if the relevant methodology requires an additionality tool, its use is required. 10

10 An example is some CDM methodologies approved by ACR.

### Table 3—Three-Prong Additionality Test

### Test Key Questions

### Regulatory Surplus

Is there an existing law, regulation, statute, legal ruling, or other regulatory framework in effect as of the project Start Date that mandates the project activity or effectively requires the GHG emissions reductions? Yes=Fail; No=Pass

### Common Practice

In the field or industry/sector, is there widespread deployment of this project, technology, or practice within the relevant geographic area? Yes=Fail; No=Pass

Choose one of the following three:

Does the project face capital constraints that carbon revenues can potentially address; or is carbon funding reasonably expected to incentivize the project's implementation; or are carbon revenues a key element to maintaining the project action's ongoing economic viability after its implementation? Yes=Pass; No=Fail

Does the project face significant technological barriers such as R&D deployment risk, uncorrected market failures, lack of trained personnel and supporting infrastructure for technology implementation, or lack of knowledge on practice/activity, and are carbon market incentives a key element in overcoming these barriers? Yes=Pass; No=Fail

Does this project face significant organizational, cultural, or social barriers to implementation, and are carbon market incentives a key element in overcoming these barriers? Yes=Pass; No=Fail

If the project passes the Regulatory Surplus and Common Practice tests, and at least one Implementation Barrier test, ACR considers the project additional.

### 1. Regulatory Surplus Test

The regulatory surplus test requires the Project Proponent to evaluate existing laws, regulations, statutes, legal rulings, or other regulatory frameworks that directly or indirectly affect GHG emissions associated with a project action or its baseline candidates, and which require technical, performance, or management actions. These legal requirements may require the use of a specific technology, require meeting a certain standard of performance (e.g., new source performance standards), or require managing operations according to a certain set of criteria or practices (e.g., forest management rules). In determining whether an action is surplus to regulations, the Project Proponent need not consider voluntary agreements without an enforcement mechanism, proposed laws or regulations, optional guidelines, or general government policies.

Projects that are deemed regulatory surplus are considered surplus for the duration of their Crediting Period. If regulations change during the Crediting Period, this may make the project non-additional and thus ineligible for renewal, but does not affect its additionality during the current Crediting Period, unless otherwise specified in the project-specific methodology.

### 2. Common Practice Test

The common practice test requires the Project Proponent to evaluate the predominant technologies or practices in use in a particular industry, sector, and/or geographic region, as determined by the degree to which those technologies or practices have penetrated the market, and demonstrate that the proposed project activity is not common practice and will reduce GHG emissions below levels produced by common technologies or practices within a comparable environment (e.g., geographic area, regulatory framework, investment climate, access to technology/financing, etc.).

The level of penetration that represents common practice may differ between sectors and geographic areas, depending on the diversity of baseline candidates. The common practice penetration rate or market share for a technology or practice may be quite low if there are many alternative technologies and practices. Conversely, the common practice penetration rate or market share may be quite high if there are few alternative technologies or practices. Projects that are “first-of-its-kind” are not common practice.

Projects that are deemed to go beyond common practice are considered beyond common practice for the duration of their Crediting Period. If common practice adoption rates of a particular technology or practice change during the Crediting Period, this may make the project non-additional and thus ineligible for renewal, but does not affect its additionality during the current Crediting Period.

Note that the common practice test, a component of the three-prong test, is distinct from a performance standard. For some but not all activities, the data used to define common practice in a particular industry, sector or region may be functionally equivalent to the data required to establish an acceptable practice based performance standard. In such cases, Project Proponents may elect the option to demonstrate additionality by defining a practice-based performance standard and demonstrating that the project activity both exceeds this standard and is surplus to regulations.

### 3. Implementation Barriers Test

An implementation barrier represents any factor that would prevent the adoption of the project activity proposed by the Project Proponent. Generally, there are no barriers to the continuation of current activities, with the exception of regulatory or market changes that force a shift in a project activity, or the end of equipment's useful lifetime.

Under the implementation barriers test, Project Proponents shall choose at least one of three barrier assessments: i) financial, ii) technological, or iii) institutional. Project Proponents may demonstrate that the project activity faces more than one implementation barrier, but are not required to address more than one barrier.
Financial—Financial barriers can include high costs, limited access to capital, or an internal rate of return in the absence of carbon revenues that is lower than the Proponent's established and documentable minimum acceptable rate. Financial barriers can also include high risks such as unproven technologies or business models, poor credit rating of project partners, and project failure risk. If electing the financial implementation barrier test, Project Proponents shall include solid quantitative evidence such as net present value (NPV) and internal rate of return (IRR) calculations.
Technological—Technological barriers can include R&D deployment risk, uncorrected market failures, lack of trained personnel and supporting infrastructure for technology implementation, and lack of knowledge on practice/activity.
Institutional—Institutional barriers can include institutional opposition to technology implementation, limited capacity for technology implementation, lack of management consensus, aversion to upfront costs, and lack of awareness of benefits.

### B. Performance Standard Approaches

In lieu of the three-prong test, ACR also recognizes the “performance standard” approach in which additionality is demonstrated by showing that a proposed project activity is (1) surplus to regulations, and (2) exceeds a performance standard as defined in an approved methodology.

Project Proponents must first establish regulatory additionality per the requirements in section A.1 of this chapter.

Second, under the performance standard approach projects are required to achieve a level of performance that, with respect to emission reductions or removals, or technologies or practices, is significantly better than average compared with similar recently undertaken practices or activities in a relevant geographic area. 11 The performance threshold may be:
Practice-based: developed by evaluating the adoption rates or penetration levels of a particular practice within a relevant industry, sector or sub-sector. If these levels are sufficiently low that it is determined the project activity is not common practice, then the project activity is considered additional. Specific thresholds may vary by industry, sector, geography and practice, and are specified in the relevant methodology.
Technology standard: installation of a particular GHG-reducing technology may be determined to be sufficiently uncommon that simply installing the technology is considered additional. Also termed a “positive list” approach.
Emissions rate or benchmark (e.g., tonnes of CO2e emission per unit of output): with examination of sufficient data to assign an emission rate that characterizes the industry, sector, subsector, or typical land management regime, the net GHG emissions/removals associated with the project activity, in excess of this benchmark, may be considered additional and credited.

Performance standard baselines specific to particular project types, activities and regions will be detailed in the relevant ACR-approved methodologies.

11 Adapted from the U.S. Environmental Protection Agency Climate Leaders offset methodologies at http://www.epa.gov/stateply/resources/optional-module.html.

### Chapter 5: Permanence

In GHG accounting, the issue of permanence arises from the potential for reversal of GHG removal enhancements. A reversal is an intentional or unintentional event that results in the emissions into the atmosphere of stored or sequestered CO2-e for which offset credits were issued. Impermanence is not an issue for some project types for which the GHG reductions or avoidance are not reversible once they occur. However terrestrial and geologic sequestration projects have the potential for GHG reductions and removals to be reversed upon exposure to risk factors, including unintentional reversals (e.g., fire, flood, insect infestation etc. for terrestrial projects; unanticipated releases of CO2 for geologic projects) and intentional reversals (e.g., landowners or Project Proponents choosing to discontinue project activities).

ACR requires that projects with a risk of reversals shall assess and mitigate risk, and monitor, report and compensate for reversals.

### A. Assessment of Risk

To assess the risk of reversals, Project Proponents of terrestrial and geologic sequestration projects must conduct a risk assessment using an ACR approved tool that addresses both general and project-specific risk factors. General risk factors include financial failure, technical failure, management failure, rising land opportunity costs, regulatory and social instability, and natural disturbances. Project-specific risk factors vary by project type.

Project Proponents shall conduct their risk assessment using the risk analysis tool specified in the applicable methodology. The output from a risk analysis tool will be a risk classification that is translated into a percentage or number of offsets that must be deposited in the ACR Buffer Pool or ACR Reserve Account to mitigate the risk of reversal (unless the Proponent elects another ACR-approved risk mitigation mechanism, if allowed by the applicable methodology).

The Project Proponent shall conduct this risk assessment and propose a corresponding buffer contribution. The risk assessment, overall risk category, and proposed buffer contribution shall be included in the GHG Project Plan. ACR evaluates the proposed overall risk category and corresponding buffer contribution. The VVB evaluates whether the risk assessment has been conducted correctly.

If no reversals occur, the project's risk category and buffer percentage (if applicable) remain unchanged for five years. The risk analysis must be re-evaluated every five years, coincident with the interval of required site visit verification. An exception is in the event of a reversal, in which case the project baseline, risk category and buffer contribution (if applicable) shall be re-assessed and re-verified immediately.

### B. Reversal Mitigation, Reporting and Compensation

Project proponents shall enter into a legally-binding Reversal Risk Mitigation Agreement with Winrock/ACR that allows them to select a reversal risk mitigation mechanism and details the requirements for reporting and compensating reversals.

### Primary Risk Mitigation Mechanism: The ACR Buffer Pool

For Project Proponents choosing the ACR buffer pool as the risk mitigation mechanism, the project contributes the number of offsets as determined by the project-specific risk assessment to a buffer account held by ACR in order to replace unforeseen losses. ACR has sole management and operational control over the offsets in the buffer pool. In the event of a reversal, ACR retires from the buffer pool an adequate number of offsets to compensate for the reversal.

To provide flexibility to Project Proponents, contributions to the buffer pool need not come from the project itself whose risk is being mitigated. Through adherence to ACR standards all ERTs are fungible, i.e., one metric ton GHG reduction or removal from any project is of equal benefit to the atmosphere as any other project. Therefore, a Project Proponent may make its buffer contribution in ERTs of any type and vintage.

Relevant sector standards (e.g., the ACR Forest Carbon Project Standard) provide further detail on the operation of the ACR buffer pool, including retirement of offsets to mitigate reversals, requirements for replenishing the buffer in the event of a reversal, return of buffer tons to the Project Proponent over time in the event of no reversals, and the possibility for buffer contributions to increase or decrease over time as a project undergoes periodic verification and re-assessment of risk.

### Alternate Risk Mitigation Mechanisms

In lieu of making a buffer contribution in project ERTs or purchased ERTs of other type and/or vintage, Project Proponents may propose an insurance product for ACR approval as a risk mitigation mechanism. Insurance may be a financial product based on an actuarial analysis of project risk, considering the region, threats, mitigating factors etc., similar to the assessment done for property insurance.

The Project Proponent may provide insurance, bonds, letters of credit or other financial assurances to ACR in amounts, and in form and substance, satisfactory to ACR in ACR's sole and absolute discretion. Such financial products must assure provision of sufficient funds to ACR, in the event a project suffers an unintentional or intentional reversal of sequestered carbon, to purchase and retire a number of ERTs sufficient to offset such reversal. There may be no hidden costs, exclusions, or unanticipated liabilities. ACR must approve the proposed alternative following due diligence by ACR at the Project Proponent's or insurance provider's expense.

### C. Monitoring for Reversals

All projects must adhere to ongoing monitoring requirements as detailed in relevant methodologies.

### D. Reversal Reporting and Compensation

Reversals must be reported and compensated for following requirements as detailed in the Reversal Risk Mitigation Agreement.

### Chapter 6: Project Development Trajectory

ACR requires every project submitted for registration to use an ACR-approved methodology. This Chapter focuses on the project development steps subsequent to methodology approval—optional listing, GHG Project Plan submission, eligibility screening, registration, validation and verification, and issuance of ERTs.

GHG Project Plans are screened by ACR against the ACR Standard, any relevant sector standard, and the relevant methodology. A successful eligibility screening results in ACR's non-binding determination that the GHG Project Plan complies with all applicable requirements. The eligibility screening does not include a detailed review of project data and does not take the place of nor reduce the scope of validation and verification by an ACR-approved independent third-party VVB. Validation and verification may occur simultaneously and must occur prior to issuance of ERTs. Upon acceptance by ACR of the verification statement, ACR registers the project, posts project documents, and issues serialized ERTs to the Project Proponent's account. The next steps (sale, retirement, etc.) are at the discretion of Project Proponents and counterparties.

A. Project Development Process A Project Proponent using an ACR-approved methodology shall proceed per the steps described below.

1. (Optional) Project Proponent lists the project with ACR by submitting a listing form. Once listed with ACR, projects must register12 on ACR within two years. If a project submits a listing form but does not register within two years, it must resubmit a listing form and update to the most recent version of the ACR Standard and applicable methodology.

2. Project Proponent submits a GHG Project Plan using the ACR-approved methodology.

3. ACR screens the GHG Project Plan, at fees per the currently published ACR fee schedule,13 against the ACR Standard, relevant sector standard, and methodology. This screening results in (a) approval to proceed to validation/verification, (b) requests for clarifications or corrections, or (c) rejection because the project is ineligible or does not meet requirements of the ACR Standard. If the ACR screening includes requests for clarifications or corrections, the Project Proponent may re-submit the GHG Project Plan for further eligibility screening. One re-submittal is allowed without

12 A project is considered to be “registered” in the ACR Registry platform upon a successful eligibility screening which means that the project may proceed to validation and verification. 13 The ACR fee schedule is posted at www.americancarbonregistry.org.

additional fee; subsequent re-submittals require an additional eligibility screening fee. Upon a successful eligibility screening, a project is considered to be registered in the ACR.

4. Having conducted the eligibility screening and received approval to proceed to validation/verification, the Project Proponent hires an ACR-approved independent third-party VVB to validate the GHG Project Plan and verify GHG assertions. Validation and verification may occur simultaneously and must occur prior to issuance of ERTs. Fees for validation and verification are as agreed between the Project Proponent and verifier. This results in submission to ACR of a validation report, verification report and verification statement.

5. ACR reviews the validation and verification documents. This results in a) acceptance, b) acceptance contingent on requested corrections or clarifications, or c) rejection. See ACR Validation and Verification Guideline for further detail.

6. Upon acceptance of the verification statement, ACR makes public the GHG Project Plan, verification statement and any other non-commercially sensitive information on the ACR registry.

7. ACR issues to the Project Proponent's account serialized ERTs for the relevant reporting period, in the amount listed in the verification statement. In the case of a terrestrial or geologic sequestration project, ACR simultaneously deposits the appropriate number of ERTs into the ACR buffer pool, if this is the risk management option chosen by the Project Proponent.

8. Next steps are at the Project Proponent's discretion—offset transfer, retirement, etc.—with activation, transaction, cancellation and retirement fees per currently published ACR fee schedule.

### B. Information in a GHG Project Plan

A GHG Project Plan is a document that describes the Project Activity; addresses ACR eligibility requirements; identifies sources and sinks of GHG emissions; establishes project boundaries; describes the baseline scenario; defines how GHG quantification will be done and what methodologies, assumptions, and data will be used; and provides details on the project's monitoring, reporting, and verification procedures. The GHG Project Plan shall use the ACR template and include the following information: □ Project title, purpose(s) and objective(s); □ Type of GHG project; □ Project location, including geographic and physical information allowing for the unique identification and delineation of the specific extent of the project; □ Physical conditions prior to project initiation; □ Description of how the project will achieve GHG emission reductions and/or removal enhancements; □ Project technologies, products, services and expected level of activity;

Ex ante projection of estimated GHG emission reductions and removal enhancements, stated in metric tons of CO2e; □ Identification of risks that may substantially affect the project's GHG emission reductions or removal enhancements; □ Roles and responsibilities, including contact information of the Project Proponent, other project participants, relevant regulator(s) and/or administrators of any GHG Program(s) in which the GHG project is already enrolled, and the entities holding offset title and land title; D Information relevant to the eligibility of a GHG project and quantification of GHG emission reductions or removal enhancements, including legislative, technical, economic, sectoral, sociocultural, environmental, geographic, site-specific and temporal information; □ Relevant outcomes from any stakeholder consultations and mechanisms for on-going communication, as applicable; □ Chronological plan for initiating project activities, project term, frequency of monitoring, reporting and verification, including relevant project activities in each step of the GHG project cycle; □ Notification of relevant local laws and regulations related to the project and a demonstration of compliance with them; □ Statement whether the project has applied for and been registered and/or been issued GHG emission reduction or removal credits through any other GHG emissions program, including detailed information on any credit issuance (volume, vintage, status), and information on any rejections of the project application, as applicable; □. An environmental and community impact assessment, following ACR requirements, to ensure compliance with best practices and that safeguard measures in place to avoid, mitigate or compensate for potential negative impacts, and how such measures will be monitored, managed and enforced.

### C. Project Deviations

ACR will permit project-specific deviations to an existing approved methodology where they do not negatively impact the conservativeness of an approved methodology's approach to the quantification of GHG emissions reductions and removal enhancements. For instance, where alternate monitoring or measurement regimes are proposed, ACR may permit these changes provided they are conservative. ACR will not permit, on a project-specific basis, changes to requirements related to additionality assessment or baseline establishment.

Project Proponents shall submit any proposed project-specific methodology deviation to ACR for review and approval. Deviations apply for that specific project but are not published as modifications to the methodology. Project Proponents must provide evidence that the proposed deviation (e.g. a substitute calculation method for missing data) is conservative, i.e. likely to underestimate net GHG reductions or removal enhancements. Project Proponents shall request a project-specific deviation by using the Methodology Deviation template available at www.americancarbonregistry.org.

### D. Project Monitoring Reports

Project monitoring reports shall be completed for each verified reporting period. The monitoring report shall describe the current status of project operation, and include the data monitored and monitoring plan, and the calculated emission reductions for the reporting period. Additionally, project monitoring reports shall describe any project-specific deviations that may have occurred during the reporting period as per the below.

Changes to validated GHG Project Plans shall not be conducted. Instead, project-specific deviations from methodology requirements or other changes from the validated GHG Project Plan (such as new GHG sources, sinks, or reservoirs) must be described in a Project Monitoring Report (and all subsequent Project Monitoring Reports) and submitted during the Project's subsequent verification. As described in Section C. above, any project-specific deviation from methodology requirements must be pre-approved by ACR. Where changes to GHG Project Plans require revisions to baseline or additionality assessments, these changes must be validated at the time of the subsequent verification. Project Proponents shall use the template for Project Monitoring Reports available at www.americancarbonregistry.org.

### E. Aggregation and Programmatic Development Approach

Overhead costs associated with carbon offset projects can sometimes make it impractical for individual project participants to access the carbon market. ACR has established procedures for projects to include multiple participants as an aggregated project or as a Programmatic Development Approach (PDA) in order to achieve efficiencies of-scale and other potential project development cost savings while preserving the accounting principles of the ACR Standard and its approved methodologies, and the integrity of the monitoring, reporting and verification processes. Streamlined processes associated with documentation, registration and verification may be available to projects applying these approaches.

### Aggregation

A Project Proponent proposing an Aggregate shall submit a GHG Project Plan encompassing all project sites, fields, parcels or facilities (hereafter referred to collectively as “sites”) with a single project Start Date and Crediting Period. Project boundaries, baseline definition, additionality demonstration, and all other requirements are applied at the level of the Aggregate, and no new sites can be added during the project Crediting Period.

The ACR Standard requirements for precision (+10% of the mean at a 90% confidence level) shall be applied at the level of the entire Aggregate for the purposes of monitoring and verification. The GHG Project Plan for an Aggregate is subject to eligibility screening by ACR and third-party validation, once per Crediting Period. If the Project Proponent anticipates adding more project sites before the end of the Crediting Period, they should instead register using the Programmatic Development Approach (PDA).

### Programmatic Development Approach

The Programmatic Development Approach (PDA) provides for organization of project participants around basic similarity criteria and a common project start date and crediting period. The PDA is intended for projects where the complete enrollment of all project participants or sites is impractical at the time of initial validation. While this approach allows for the enrollment of new project participants and sites over time, it does require more complex project management and verification considerations than an aggregated project approach where all project participants and sites are included in the project's initial validation.

### General PDA Requirements:

□ A PDA project will be under the management of a single Project Proponent and registered by a single ACR account holder; □ Participating sites, fields, parcels or facilities (hereafter referred to collectively as “sites”), must implement the same ACR approved methodology, meet all project eligibility, boundary, baseline and additionality criteria and will have the same overarching project start date and crediting period; □ The GHG Project Plan shall specify the programmatic boundaries (geographic, temporal, and GHG assessment boundary), a baseline scenario for the initial cohort(s), as detailed below, a monitoring/verification plan for the initial cohort(s), and a planned recruitment schedule for future cohorts which will be added to the project; □ The Project Proponent must describe in the GHG Project Plan a management system that includes the following: ∘ The reason why all project participants and sites cannot be included upon initial validation; o A clear definition of the roles and responsibilities of personnel involved in the process of inclusion of new cohorts; ∘ Procedures for QA/QC of inclusion of new cohorts conducted by project proponents, made available to the VVB at the time of validation of the PDA project; ∘ A procedure to avoid double counting that no site or cohort has been or will be registered on ACR as its own project, or in a cohort of another PDA project; and ∘ A records and documentation control process for each cohort under the PDA, made available to the VVB at the time of request for inclusion of the cohort; and □ Each cohort must undergo validation and verification by an ACR approved VVB before ERTs can be issued for its associated project activities. Cohorts added after the project's initial validation shall be validated during the project's next full verification, and must include site visits to a sample of new sites within each cohort, according to the VVB's risk-based sampling plan.

Each cohort shall:

□ Be a grouping of project participants, implementing eligible project practices or technologies as specified in a single module or methodology, meeting all eligibility, project boundary, and additionality criteria of a PDA project; □ Be defined in a Cohort Design Document including a delineation of the cohort's geographic and temporal boundaries, including all of the project participants and participating sites □ Include sites implementing the same practices/technologies or suite of practices/technologies in a single approved methodology; ∘ For PDA projects using a modular methodology, cohorts shall be comprised of project participants implementing the same practices/technologies or suite of practices/technologies in a single approved module, unless otherwise stated in the methodology; □ Have a single validation and verification schedule for all sites within a cohort; □ Use a comparable quantification approach for the baseline and project conditions respectively (models, equations, measurements, default factors) for each project activity included in the cohort as outlined in the approved module or methodology. These methods must be documented in the GHG Project Plan (for the initial cohort(s)) and a Cohort Design Document (for each subsequent cohort added to the project); □ For AFOLU PDA projects only: be located within a pre-defined geographic region, such that all sites within the cohort are located within a maximum of three ecoregions, which are defined by the World Wildlife Foundation (2014) as “A large unit of land or water containing a geographically distinct assemblage of species, natural communities, and environmental conditions. The boundaries of an ecoregion are not fixed and sharp, but rather encompass an area within which important ecological and evolutionary processes most strongly interact”14; ∘ To determine the ecoregion of each participating site located in the U.S., please refer to the U.S. Forest Service maps found at the link provided in the footnote below15; ∘ To determine the ecoregion of each international participating site outside of the U.S., please refer to the World Wildlife Federation delineation of ecoregions found at the link provided in the footnote below16; □ Provide confirmation of compliance with any applicable environmental impact analysis requirements (if required by the methodology), unless the analysis was undertaken for the whole PDA project and applies equally to each cohort; □ Provide information as applicable on how the public stakeholder consultation process was conducted, a summary of any comments received and how due account was taken of any comments received, unless the comments were sought for the whole PDA project and apply equally to each cohort; and □ Meet the ACR requirements for precision (for methodologies requiring direct measurements—+10% of the mean at a 90% confidence level), which shall be applied at the cohort-level for the purposes of monitoring and verification.

Each site, field, parcel or facility (collectively the “sites”) participating in a PDA project must:

□ Be assigned to a cohort prior to validation of that cohort; □ Be available for a site visit during the validation and any subsequent verification where site visits are required. VVBs may use equal probabilities amongst sites, fields or parcels to select those receiving verification site visits, or a risk- or sensitivity-based analysis to identify those sites with the strongest influence over a project's overall carbon reduction estimates. VVBs must use their own discretion to determine if a cohort requires sub-sampling. All project sites and cohorts are subject to desk based review at minimum; □ Be described in a Cohort Design Document outlining the unique attributes of each site, to include each of the following: ∘ Geographic information to uniquely identify the site including maps and spatial files; ∘ Name/contact details of the entity/individual responsible for the operation of each site; ∘ The site-specific Implementation Date and confirmation that the Implementation Date of any site is not, or will not be, prior to the Project's start date; ∘ Information on how the site fulfills the eligibility criteria, is within the project boundaries, and demonstration of additionality as specified in the GHG Project Plan; and ∘ Information about the site's eligibility, ownership of emission reductions, land cover or crop type, etc.

### F. Commercially Sensitive Information

Project Proponents may designate certain parts of the GHG Project Plan or other project documentation as Commercially Sensitive Information. This information must be available for review by ACR and the VVB (with non-disclosure agreements as necessary), but will be excised from the project documentation posted publicly on the ACR registry.

For the sake of transparency, ACR shall presume project information to be available for public scrutiny, and demonstration to the contrary shall be incumbent on the Project Proponent. At a minimum, ACR shall disclose publicly the project baseline scenario, calculations, monitoring report and additionality assertion. The VVB shall check that any information requested as “commercially sensitive” meets the ACR definition of Commercially Sensitive Information.

### G. Additional Required Documentation for Eligibility Screening

ACR may require the following documentation as part of screening the GHG Project Plan: □ Title documents or sample landowner agreements; □ Chain of custody documentation, if applicable; □ ACR-Proponent agreement governing buffer pool obligations, if applicable.

Proof of title shall accompany each GHG Project Plan, and shall contain one or more of the following: a legislative right; a right under local common law; ownership of the plant, land, equipment and/or process generating the reductions/removals; or a contractual arrangement with the owner of the plant, land, equipment or process that grants offset title to the Project Proponent.

Project Proponents shall include documentation to establish chain of custody, prior to registration on ACR, if the project offsets have been bought and sold previously, or if the project has a forward option contract. Examples of appropriate documents are: □ Delivery of Confirmation Notice; □ Emissions Reduction Purchase Agreement; □ Signed Attestation of Ownership; □ Forward Option Purchase Agreement.

### H. Crediting Period Renewal

All projects have a limited Crediting Period, i.e., the finite length of time for which a GHG Project Plan is valid, and during which a project can generate offsets against its baseline scenario.

In general, the Crediting Period for non-AFOLU projects is ten (10) years, unless otherwise specified in the relevant ACR sector standard or approved methodology. Crediting periods for AFOLU projects vary and are specified in the relevant sector standard and/or methodology.

A Project Proponent may apply to renew the Crediting Period by: □ Re-submitting the GHG Project Plan in compliance with then-current ACR standards and criteria; □ Re-evaluating the project baseline; □ Demonstrating additionality against then-current regulations, common practice and implementation barriers or against an approved performance standard and then-current regulations; □ Using ACR-approved baseline methods, emission factors, tools and methodologies in effect at the time of Crediting Period renewal; and, □ Undergoing validation and verification, as required.

ACR does not limit the allowed number of renewals, since at each Crediting Period renewal the Project Proponent must demonstrate that the project is additional and meets all ACR requirements. An acceptable validation report is necessary in order for ACR to renew the Crediting Period and continue issuing offsets generated by the project. Upon acceptance by ACR of the validation and verification documents, ACR will issue new ERTs each year (or more or less frequently, at Proponent's request) for the duration of the new Crediting Period, provided the Proponent submits its Annual Attestation, periodic desk-based verifications, and full verifications at least every five years.

On a project level, project proponents are required to meet the requirements of the most recent version of the ACR Standard. When a project seeks renewal of a crediting period (i.e. the previous was previously validated under a prior version of the ACR standard and the project's crediting period has expired), the project is required to meet the requirements of the most recent version of the ACR Standard.

### Chapter 7: Methodologies and Tools

If ACR has not yet published a methodology for a particular project type, the Project Proponent has the option to request approval of a methodology developed under another GHG program, or to submit a new or modified methodology to ACR for approval. Any project proposing to use an ACR-approved methodology from another GHG program must comply with the ACR Standard and any relevant ACR sector standard.

### A. GHG Measurement Tools and Methodologies

1. ACR-Published and CDM-Approved Methodologies

Methodologies published by ACR via the public consultation and peer review process are approved without qualification. Methodologies approved by the CDM Executive Board are generally approved for use in non-Annex I countries; however, Project Proponents implementing projects under CDM methodologies in the U.S. or other Annex I country must have ACR's review, clarifications and approval first to ensure compliance with ACR standards.

2. Modifications to Existing Approved Methodologies ACR may permit modifications where they do not negatively impact the conservativeness of an approved methodology's approach to determining additionality and quantification of GHG emissions reductions and removal enhancements. Methodology modifications may be submitted for review by ACR, at fees per the currently published ACR fee schedule, ACR will review the extent of the modification and make a determination whether the internal review, public consultation, and peer review process, as described in Section B, must be implemented. In general, if the extent of the proposed modification(s) necessitates the process described in Section B, a new version number for the methodology will be issued (i.e. Version 3.0 to Version 4.0). Modifications to eligibility, applicability, project activities, and/or baseline assumptions are likely to trigger the full process stipulated in Section B. Minor modifications to correct quantification errors or provide clarification on monitoring requirements may not require the full process stipulated in Section B.

3. New Methodologies New methodologies proposed to ACR for approval always require internal screening, public consultation and blind scientific peer review as described in section B.

### B. ACR's Internal Review, Public Consultation and Scientific Peer Review Process

The following process is applied to new methodologies and certain methodology modifications. In such cases, ACR coordinates a process of internal review, public stakeholder consultation and a blind scientific peer review. The process is administered by ACR, with fees charged to the methodology author.

1. The Project Proponent submits the proposed new or modified methodology to ACR. ACR has templates posted at www.americancarbonregistry.org for some proposed methodologies. Where ACR has a posted template, Project Proponents must submit their proposed methodology using this template in order to reduce the time and cost of the approval process for both Project Proponent and ACR.

2. ACR screens the methodology against ACR requirements, communicates any corrections or clarifications that are immediately needed, and informs the methodology author of its judgment whether the methodology is ready for public consultation and peer review. ACR conducts this internal review at currently published fees. If the methodology author elects to proceed, the methodology author addresses any corrections and clarifications identified in the ACR review and resubmits the methodology.

3. ACR coordinates a public consultation process. The methodology is posted publicly on the ACR website for a minimum of 30 days and ACR sends out a public notice inviting comments. During this period, the methodology authors may also elect to conduct a webinar with ACR to present the draft methodology and solicit additional comments. At the conclusion of the public comment period, ACR compiles all comments by methodology section and forwards a complied report to the methodology author. The methodology author incorporates revisions and/or documents responses to each comment, which are posted on ACR's website.

4. The revised methodology is provided to a team of independent subject matter experts for a blind scientific peer review process. ACR may consult the relevant ACR Technical Committee in the selection of reviewers. The lead reviewer compiles comments and recommendations from the peer review team, and prepares a summary report. ACR delivers to the methodology author a peer review report, organized by section of the methodology, to which the methodology author must respond by incorporating revisions and/or documenting justifications for the proposed approach. Generally, several rounds of peer review are necessary. Timing and cost of peer review depends on the complexity, scope and quality of the methodology and the availability of peer reviewers. The cost of peer review is borne by the methodology author.

5. Once all required corrections have been made, ACR approves the new methodology and publishes it on the ACR website. An approved methodology may be used by any Project Proponent, including but not limited to the methodology author, in preparing GHG Project Plans and registering projects on ACR.

6. ACR posts process documentation—including all public comments and documented responses, and all peer review comments and documented responses—along with the public comment version of the methodology, and the final approved methodology.

Scientific peer review teams are selected from a pool of potential reviewers with applicable subject matter expertise. ACR actively identifies and qualifies candidates for inclusion in this pool, and also publicly solicits applications from interested parties. Applications are reviewed for sector expertise, GHG quantification experience, and impartiality. Throughout and after the peer review process, the experts selected for each review team remain unknown to the methodology author as well as the public.

### C. Updates to ACR-Approved Methodologies and Tools

From time to time ACR may update ACR-Approved Methodologies and Tools. Such updates occur when significant changes to GHG accounting best practice or the legislative and/or regulatory context justify an update; when sufficient new data is available to revise eligibility and/or additionality requirements; when ACR becomes aware of clarifications that should be made; or for other reasons.

For methodologies that employ a performance standard for additionality assessment, ACR shall review the validity and underlying assumptions of the performance standard for all projects except forestry every five years, at minimum and for forestry projects every ten years, at minimum.

### D. Roles of the ACR Technical Committee(s)

ACR from time to time may establish Technical Committees for particular sectors (e.g., AFOLU), to provide independent advice to ACR on methodology acceptance, methodology modifications and project deviations, selection of peer reviewers, and related issues. The responsibilities of the Technical Committees include, but are not limited to:

□ Review proposed new methodologies and tools submitted to ACR for approval. □ Advise ACR on the selection of appropriate peer reviewers for a proposed new methodology or methodology revision. O Make final determinations in the event consensus on a particular methodological issue is not reached by the peer review team or between the peer reviewers and the methodology author. O Advise ACR on continuous improvements to its AFOLU standards, including issuance of new versions at appropriate intervals. □ Advise ACR on decisions to commission new methodologies and tools using internal resources.

ACR Technical Committees are constituted via calls for applications to select the most relevant experts.

### Chapter 8: Environmental and Community Safeguards

ACR supports a broad and diverse set of offset project activities, each of which has its own potential to generate both positive and negative environmental and social impacts. Positive impacts can contribute to sustainable development objectives, and negative risks and impacts can be identified, evaluated and managed through appropriate safeguard procedures.

ACR requires that projects adhere to environmental and community safeguards best practices to: □ Ensure that projects “do no harm” by maintaining compliance with local, national and international laws and regulations; □ Identify environmental and community risks and impacts; □ Detail how negative environmental and community impacts will be avoided, reduced, mitigated or compensated and how mechanisms will be monitored, managed and enforced; □ Ensure that the rights of impacted communities and other stakeholders are recognized, that they have been fully and effectively engaged and consulted; □ Ensure that ongoing communications and grievance redress mechanisms are in place, and that impacted communities will share in the project benefits.

### Environmental and Community Impact Assessment Requirements

ACR requires that all projects prepare and disclose as part of the GHG Plan an environmental and community impact assessment. ACR does not require that a particular process or tool be used for the impact assessments as long as basic requirements are addressed, as detailed below. ACR projects can follow internationally recognized approaches such as The World Bank Safeguard Policies or can be combined with the Climate Community and Biodiversity Alliance (CCBA) Standard or the Social Carbon Standard for the assessment, monitoring and reporting of environmental and community impacts.

Environmental and community impacts of projects should be net positive. Project Proponents shall include in their GHG Project Plan a description of impacts of the project on communities and the environment in the immediate project area. This shall include changes in community well-being due to the Project Activity and an evaluation of any negative impacts on community groups. Project Proponents shall base these estimates on defined and defensible assumptions about how the Project Activity will alter social and economic well-being, including potential impacts of changes in natural resources and ecosystem services identified as important by the communities for the project duration.

The assessment should include the following:

1. An overview of the project activity and geographic location 2. Applicable laws, regulations, rules and procedures and the associated oversight institutions 3. A description of the process to identify community(ies)17 and other stakeholders18 impacted by the project and, as applicable, the community consultation and communications plan 4. An assessment of the project's environmental risks and impacts including but not limited to factors such as climate change mitigation and adaptation, biodiversity, air quality, water quality, soil quality, ozone quality, as well as the protection, conservation or restoration of natural habitats such as forests, grasslands and wetlands. The assessment shall: 1) identify each risk/impact 2) categorize the risk/impact as positive, negative or neutral and substantiate the risk category; 3) describe how any negative impacts will be avoided, reduced, mitigated or compensated; 4) detail how risks and impacts will be monitored, how often and by whom; and 5) (Optional) describe how positive impacts contribute to sustainable development goals. 5. For any community-based project, an assessment of the project's community risks and impacts including but not limited to factors such as land and natural resource tenure, land use and access arrangements, natural resource access (ie: water, fuelwood), food security, land conflicts, economic development and jobs, cultural heritage and relocation. The assessment shall: 1) briefly describe the process to identify community risks/impacts, 2) identify each risk/impact, 3) categorize the risk/impact as positive, negative or neutral and substantiate the risk category; 4) provide detailed information regarding the community stakeholder consultation process (meeting minutes, attendees), including documentation of stakeholder comments and concerns and how those are addressed; 5) provide evidence of Free, Prior and Informed Consent (FPIC) for the project activity, as applicable, 6) provide evidence of no relocation or resettlement (voluntary or involuntary), as applicable, 7) describe how any negative project impacts will be avoided, reduced, mitigated or compensated; 8) detail how risks and impacts will be monitored, how often and by whom; 9) describe the mechanism for ongoing communications with the community and grievance mechanisms, as applicable, and 10) (Optional) describe how positive impacts contribute to sustainable development goals 17 A community as defined by CCBA includes all groups of people including indigenous peoples, mobile peoples and other local communities, who live within or adjacent to the project area as well as any groups that regularly visit the area and derive income, livelihood or cultural values from the area. This may include one or more groups that possess characteristics of a community, such as shared history, shared culture, shared livelihood systems, shared relationships with one or more natural resources (forests, water, rangeland, wildlife, etc.), and shared customary institutions and rules governing the use of resources 18 Other Stakeholders are defined as groups other than Communities who can potentially affect or be affected by the project activities and who may live within or outside the Project Zone.

### Ongoing Disclosure and Enforcement

Project Proponents shall disclose in their Annual Attestations to ACR any negative environmental or community impacts or claims of negative environmental and community impacts and the appropriate mitigation measure.

ACR reserves the right to refuse to register or issue credits to a project based on community or environmental impacts that have not or cannot be mitigated, or that present a significant risk of future negative environmental or community impacts.

### Chapter 9: Validation and Verification

This chapter provides a general overview of ACR requirements for validation of GHG Project Plans, and ex post verification of GHG assertions, by a competent and independent third-party VVB approved by ACR. Further detail on ACR verification requirements is included in the ACR Validation and Verification Guideline, available at www.americancarbonregistry.org.

### A. Definitions

ACR conducts a detailed screening of every GHG Project Plan against applicable requirements of the ACR Standard, relevant sector standard and methodology. ACR may request clarifications and corrections regarding GHG Project Plan documentation before allowing a project to commence validation. Validation is the systematic, independent and documented process for the evaluation of a GHG Project Plan against applicable requirements of the ACR Standard, sector standard and approved methodology.

Verification is the systematic, independent and documented assessment by a qualified and impartial third party of the GHG assertion for a specific reporting period.

Validation and verification must be conducted by an ACR-approved independent third-party VVB. Validation and verification may be conducted by the same entity, and may occur simultaneously.

### B. Materiality Threshold

A material misstatement is an inaccurate assertion of an offset project's GHG emission reductions/removals, which may reasonably be expected to influence decisions or actions taken by the users of the GHG project information. To accept a verification statement, ACR requires that discrepancies between the emission reductions/removal enhancements claimed by the Project Proponent and estimated by the VVB be immaterial, i.e. be less than ACR's materiality threshold of +5%. Individual or aggregation of errors or omissions greater than the ACR materiality threshold of +5% require re-stating before a verification statement will be accepted.

The below equation is to be used in order to calculate the percent error in an emission reduction assertion:

% Error=(Project Emission Reduction Assertion−Verifier Emission Reduction Recalculation Verifier Emission Reduction Recalculation)×100

### C. Validation and Verification Interval

Validation of the GHG Project Plan only occurs once per Crediting Period. Renewal of the Crediting Period requires a new validation. Per Section 6.D, if project-specific changes that require revision to baseline or additionality assessments occur after the initial validation, these changes must be disclosed in the Project Monitoring Report and validated at the Project's subsequent verification.

ACR requires verification of GHG assertions at specified intervals in order to issue new ERTs19. ERTs may be created and issued annually, or at the Proponent's request, more or less frequently. At each request for issuance of new ERTs, the Project Proponent must submit a verification statement from an approved verifier. No less than once every five years, Proponents must submit a verification statement based on a full verification including a field visit to the project site20. This five year verification requirement begins on the date that the project is registered in the ACR. The scope of this verification should include (in the case of AFOLU projects) an updated assessment of risk of reversal and an updated buffer determination, as applicable.

### D. Validation/Verification Body Requirements

Verification is a risk-based process carried out in conformance with ISO 14064-3:2006 and ISO 14065:2007.21 VVBs shall be accredited for project validation and verification in the sector of the applicable methodology, and shall meet the competence requirements as set out in ISO 14065:2007.

All VVBs must be approved by ACR and accredited under ISO 14065 by the American National Standards Institute (ANSI); or be accredited by the UNFCCC as Accredited Independent Entities approved under Joint Implementation or Designated Operational Entities approved under the Clean Development Mechanism.

A list of currently approved VVBs and the sectors for which they are approved to conduct validation and/or verification is provided at http://americancarbonregistry.org/carbon-accounting/verification.

In order to conduct validation or verification, all VVBs must be in good standing; have completed the application process described at http://americancarbonregistry.org/carbon-accounting/verification, including submitting an application form and Attestation of Validation/Verification Body which details requirements for conflicts of interest and makeup of the verification teams; document technical capabilities for each of the sectoral scopes in which the verifier seeks to conduct validation or verification; and have submitted for ACR's approval a project-specific Conflict of Interest Form.

19 Verification activities may begin only after the completion of a project's reporting period. 20 A field visit must occur at the first verification for the project. 21 ISO 14065:2007 references to ‘GHG Programme’ shall mean the American Carbon Registry.

### E. Verification Report and Statement

On completion of verification, the Project Proponent shall submit a verification report and verification statement to ACR. Verification documents shall be in English. They shall describe the verification process, any issues raised during the verification and their resolutions, and the conclusions reached by the VVB. The verification report shall:

• Describe the level of assurance of the verification statement; • Describe the objectives, scope and criteria of the verification against the ACR Standard and relevant sector standards; • Describe whether the data and information supporting the GHG assertion were hypothetical, projected and/or historical in nature; • State the actual number of ERTs associated with the project-specific monitoring report that the verifier has verified; • Include the GHG assertion, signed by the lead verifier; • Include the verifier's conclusion on the GHG assertion, with any qualifications or limitations; • For projects requiring Project Proponents to assess risk of reversal and apply an ACR-approved risk reversal mitigation option, include the verifier's opinion on the risk assessment and adequate risk reversal mitigation.

More detail on contents of the verification report and statement is provided in the ACR Validation and Verification Guideline.

The VVB shall keep all documents and records in a secure and retrievable manner for at least two years after the end of the relevant project Crediting Period, even if it does not carry out verification throughout the project Crediting Period.

### F. Verification Acceptance

ACR will review and accept, request corrections or clarifications, or reject the verification report and statement. If ACR requests corrections or clarifications, the Project Proponent and verifier shall make all necessary corrections and clarifications and resubmit the verification statement.

If ACR accepts a verification statement, and has already completed all other required steps, then ACR will register the project; post the GHG Project Plan, verification report and statement, and other documentation to the ACR website; and issue ERTs to the Project Proponent's account.

Projects must be verified without reservation, with Project Proponents having addressed all clarifications and corrections required by the verifier. ACR reserves the right to accept or reject verification from an approved VVB.

### G. Rotation of Verification Bodies

ACR requires that Project Proponents utilize a different VVB at a minimum of every five years or five verifications, whichever comes first.

### H. Validation and Verification Body Oversight

In addition to the accreditation processes that all ACR VVB's must adhere to, ACR reserves the right to conduct oversight activities during validation and/or verification performance by the VVB's operating under the ACR program. Oversight activities are conducted to ensure an adequate level of quality control and are intended to supplement accreditation body oversight and audit processes. Oversight activities conducted by ACR representatives include the following:

□ Review of information and supplementary documentation submitted by VVBs regarding project specific conflict of interest determinations; □ Review of VVB documentation such as verification and sampling plans; □ Review of validation and verification reports and verification statements; □ Project-level audits.

Should a project be selected by ACR for a project-level audit, the VVB must include ACR on communications with the project proponent, include ACR in substantive meetings with the project proponent, and make project-level data and information subject to validation and/or verification available to ACR for review. During a project-level audit, ACR may choose to send, at ACR expense, a representative to the validation and/or verification site visit to observe on-site verification activities. At the conclusion of a project-level audit, ACR will communicate its observations via written report directly to the VVB. The report will document, as applicable, any items of concern noted during validation and/or verification performance including areas for improvement and non-conformities with ACR validation and verification procedures.

### Chapter 10: Linkages to Other GHG Programs & Registries, Emission Trading Systems and National or Sectoral GHG Emissions Reduction Targets

### A. Policies to Prevent Double Issuance, Double Use and Double Selling of Offsets

### Projects Registered on ACR and Other Voluntary or Compliance GHG Programs or Registries

ACR allows for offset project Registration simultaneously on ACR and other voluntary or compliance GHG programs or registries only in cases where: 1) The simultaneous Registration is disclosed and approved by both programs/registries, including explicitly through regulation; and 2) Offsets issued for the same unique emissions reductions do not reside concurrently on more than one registry.

To prevent double issuance, double use and double selling of offsets for projects registered simultaneously on ACR and another GHG program: 1) Offsets representing the same emissions reduction must be publicly canceled from one registry before they can be converted and re-issued on another registry; or 2) Offsets can be issued to a project by both programs as long as the registration of the project under more than one program is disclosed to the GHG Program and the verifier, and the offset represents unique emissions reductions in terms of location (project boundary) and/or vintage.

### Transferred Projects Previously Registered on ACR and Other Voluntary or Compliance GHG Programs or Registries

For projects transferring from another GHG program to ACR, the project must be validated and verified by an ACR approved VVB to comply with the ACR Standard and relevant methodology. To avoid double issuance, double-use and double selling of the same GHG reduction or removal, any offsets that had been issued that were not transferred, sold or retired must be canceled from the other program's registry before conversion and re-issuance by ACR.

For projects transferring from ACR to another GHG program, Project Proponents must cancel from ACR all offsets that have not been transferred, sold or retired, in order to allow for conversion and re-issuance of offsets by the other GHG program on its registry.

### B. Policies to Prevent Double Claiming of Emissions Reductions

Double claiming may occur if the climate benefit of a specific GHG emissions reduction is declared by more than one entity. While this may be a concern if the emissions reduction is double-sold (monetized by both parties claiming the reduction), double claiming does not always raise environmental concerns.

For example, no environmental integrity concern is raised in the case that an offset is sold and the emissions reduction is claimed and counted by both a buyer resident in a given host country and the host country itself towards a national emission reduction target. The emission reduction occurred in the host country, and therefore there is no issue with regard to accounting for that reduction towards the national target as long as the host country is not monetizing or trading the reduction to another country towards its national target.

In the event that specific emissions reductions are being transferred or sold from the host country to another entity or country to count towards its emissions reduction targets, the opportunity for double claiming exists. Accounting procedures at both the national and international level must be in place to track emissions reductions sold to buyers towards meeting their targets. In these instances, any applicable emissions reductions can be added back in to the host country national inventory.

ACR requires that project offsets that are being sold or transferred from the host country for use towards a national or sector-wide emissions reduction target obtain a written acknowledgement from the project host country UNFCCC National Focal Point22 in order to avoid double claiming of the emissions reductions. Receipt of satisfactory acknowledgement shall be indicated on the Registry System. C. Previous Rejection by a GHG System

ACR may consider a project rejected by other voluntary or compliance GHG programs, due to procedural or eligibility requirements, if the project complies with all aspects of the ACR Standard and any relevant sector standard. The Project Proponent for such a project shall: 1) Include a statement in the GHG Project Plan that lists all other programs to which the Project Proponent has applied for registration, was rejected, and the reason(s) for the rejection. Such information shall not be considered Commercially Sensitive Information; and 2) Provide the actual rejection document(s), including any additional explanation, to ACR and its verifier.

### REFERENCES

Clean Development Mechanism (CDM). List of Accepted Baseline and Monitoring Tools and Methodologies. http://cdm.unfccc.int/methodologies/PAmethodologies/approved.html
Clean Development Mechanism (CDM). Tool for the demonstration and assessment of additionality. http://cdm.unfccc.int/methodologies/PAmethodologies/tools/am-tool-01-v5.2.pdf.
Climate, Community & Biodiversity Alliance (CCBA). Climate, Community and Biodiversity Standards, Project Design Standards, Second Edition (2008). http://www.climatestandards.org/standards/pdf/ccb_standards_second_edition_december_2008.pdf.
International Organization for Standardization (ISO) 14064-1:2006(E)—Greenhouse gases. Part 1: Specification with guidance at the organization level for quantification, monitoring, and reporting of greenhouse gas emission reductions or removal.
International Organization for Standardization ISO) 14064-2:2006(E)—Greenhouse gases. Part 2: Specification with guidance at the project level for quantification, monitoring and reporting of greenhouse gas emission reductions or removal enhancements.
International Organization for Standardization (ISO) 14064-3:2006(E)—Greenhouse gases. Part 3: Specification with guidance for the validation and verification of greenhouse gas assertions.
International Organization for Standardization (ISO) 14065:2007(E)—Greenhouse gases. Requirements for greenhouse gas validation and verification bodies for use in accreditation or other forms of recognition.
Intergovernmental Panel on Climate Change (IPCC). Fourth Assessment Report. http://www.ipcc.ch/pdf/assessment-report/ar4/syr/ar4_syr.pdf
United States Environmental Protection Agency (USEPA) Climate Leaders Program, GHG Inventory Protocol (May 2005). http://www.epa.gov/climateleaders/resources/inventory-guidance.html
World Bank. 2012. Safeguard Policies. http://go.worldbank.org/WTA1ODE7T0.
World Resources Institute and World Business Council for Sustainable Development (WRI/WBCSD), Greenhouse Gas Protocol Initiative. The GHG Protocol: A Corporate Accounting and Reporting Standard—Revised Edition (March 2004). http://www.ghgprotocol.org/files/ghg-protocol-revised.pdf.

### APPENDIX A: DEFINITIONS

Additionality ACR's additionality requirements are intended to ensure that project offsets are in addition to reductions and/or removals that would have occurred in the absence of the Project Activity and without carbon market incentives. A Project Proponent must demonstrate that the GHG emission reductions and removals associated with an offset project are above and beyond the “business as usual” scenario. ACR requires that every project either pass an approved performance standard and a regulatory additionality test, or pass a three-pronged test to demonstrate that the project activity is beyond regulatory requirements, beyond common practice, and faces at least one of three implementation barriers.

Aggregate The grouping of multiple project instances, fields, producers or facilities into a single project registered on ACR. An Aggregate must be coordinated by a Project Proponent (public or private entity) serving as the aggregator. The GHG Project Plan will define the overall project boundary and baseline conditions encompassing all project instances, fields, producers or facilities. An Aggregate will have a single Start Date and Crediting Period.

Agriculture, Forestry and Other Land Use (AFOLU) A broad category of ACR-eligible project activities that reduce GHG emissions and/or enhance GHG removals through changes in agriculture, forestry and land-use practices.

American Carbon Registry® (ACR) A leading carbon offset program founded in 1996 as the first private voluntary GHG registry in the world, ACR operates in the voluntary and regulated carbon markets. ACR has unparalleled experience in the development of environmentally rigorous, science-based offset methodologies as well as operational experience in the oversight of offset project verification, registration, offset issuance and retirement reporting through its online registry system.

ACR-approved Methodology ACR-approved methodologies include those published by ACR after public consultation and scientific peer review, and methodologies approved for use by the CDM Executive Board provided they are implemented in developing countries or otherwise have ACR approval for use in the U.S. or other Annex I nation. Methodologies approved by other GHG programs may be submitted to ACR for approval through the public consultation and scientific peer review process.

Annual Attestation Statement The statement that a Project Proponent provides annually to ACR relating to the continuance, ownership, and community and environmental impacts of a project. The Attestation is required in order to continue crediting.

Baseline Scenario The project baseline is a counterfactual scenario that forecasts the likely stream of emissions or removals to occur if the Project Proponent does not implement the project, i.e., the “business as usual” case. It also reflects the sum of the changes in carbon stocks (and where significant, N2O and CH4 emissions) in the carbon pools within the project boundary that would occur in the absence of the Project Activity.

Buffer Pool ACR risk mitigation mechanism whereby the Project Proponent contributes an adequate number of ERTs to a buffer pool managed by ACR to replace unforeseen losses in carbon stocks. The buffer contribution is a percentage of the project's reported offsets, determined through a project-specific assessment of the risk of reversal. The buffer contribution may be made in ERTs of any type and vintage.

Carbon Dioxide Carbon dioxide (CO2) is a chemical compound comprising two oxygen atoms bonded to a single carbon atom, and is the primary greenhouse gas implicated in global warming.

Carbon Dioxide-equivalent (CO2e) Carbon dioxide equivalence (CO2e) is a metric to compare GHGs based on their global warming potential (GWP) relative to CO2 over the same timeframe. The Intergovernmental Panel on Climate Change publishes GWP values for converting all GHGs to a CO2e basis.

Carbon Offset A carbon offset, also referred to as a carbon credit or offset credit, is a reduction, removal, or avoidance of GHG emissions that is used to compensate for GHG emissions that occur elsewhere. In a regulated market offsets are GHG reductions from projects undertaken outside the coverage of a mandatory emissions reduction system for which the ownership of verifiable GHG emission reductions can be transferred and used by a regulated source to meet its emission reduction obligations.”23 The ACR registers both voluntary market and compliance-eligible offsets.
23 Adapted from Pew Center on Global Climate Change. Climate Change 101: Cap and Trade. http://www.pewclimate. org/docUploads/Cap&Trade.pdf.

Carbon Pool A reservoir of carbon that has the potential to accumulate or lose carbon over time. Common forest carbon pools are aboveground biomass, below ground biomass, litter, dead wood, soil organic carbon, and wood products.

Carbon Stocks Carbon stocks represent the measured, estimated or modeled quantity of carbon held in a particular carbon pool. Quantifying GHG emissions and removals for terrestrial carbon offset projects involves estimating, for the baseline vs. project scenario, changes over time in carbon stocks in relevant pools.

Cohort A group of Project Participants (participating sites, fields, parcels or facilities), meeting all eligibility, project boundary, baseline and additionality criteria of project and sharing the same implementation start date within a project under a Programmatic Development Approach (PDA).

Clean Development Mechanism (CDM) The CDM allows GHG emission reduction and removal projects in developing countries to earn certified emission reduction (CER) credits, each equivalent to one metric ton of CO2, which can be sold and used by industrialized countries to a meet a part of their emission reduction targets under the Kyoto Protocol. The CDM is intended to stimulate sustainable development and emission reductions, while giving industrialized countries flexibility in how they meet their emission reduction targets.24

Commercially Sensitive Information Trade secrets, financial, commercial, scientific, technical or other information whose disclosure could result in a material financial loss or gain, prejudice the outcome of contractual or other negotiations, or otherwise damage or enrich the person or entity to which the information relates.

Community A community includes all groups of people including indigenous peoples, mobile peoples and other local communities, who live within or adjacent to the project area as well as any groups that regularly visit the area and derive income, livelihood or cultural values from the area. This may include one or more groups that possess characteristics of a community, such as shared history, shared culture, shared livelihood systems, shared relationships with one or more natural resources (forests, water, rangeland, wildlife, etc.), and shared customary institutions and rules governing the use of resources.25
24 http://cdm.unfccc.int/about/index.html. 25 CCB Standards—Project Design Standards. Second Edition (2008). Climate, Community & Biodiversity Alliance.

Community and Environmental Impacts Community and environmental impacts are the effects, both positive and negative, that the Project Activity may have on the socioeconomic well-being of affected communities or environmental quality in the project area. ACR requires that the Project Activity provide net benefits to affected communities and the environment, that negative impacts be mitigated or compensated and monitored throughout the project.

Crediting Period Crediting Period is the finite length of time for which a GHG Project Plan is valid, and during which a project can generate offsets against its baseline scenario. The baseline must be re-evaluated in order to renew the Crediting Period. ACR sector standards and methodologies specify Crediting Period for particular project types.

De Minimis The ACR sets a de minimis threshold of 3% of the final calculation of emission reductions or removals. For the purpose of completeness, any decreases in carbon pools and/or increases in GHG emission sources that exceed the de minimis threshold must be included. Any exclusions using the de minimis principle shall be justified using fully documented ex ante calculations.

Do no harm Offset projects must be in compliance with applicable local, national and international laws and regulations.

Double Claiming Double claiming may occur if the environmental benefit of a specific unit GHG emissions reduction or removal is counted towards more than one national or sector-wide emissions reduction target. Accounting procedures at both the national and international level must be in place to track emissions reductions sold to buyers towards meeting their targets. In these instances, any applicable emissions reductions can be added back in to the host country national inventory.

Double Issuance An instance in which a specific unit is issued more than once for the same emissions reduction or removal. This can be avoided by having preventative program rules and oversight processes in place, such as cancellation of units by one program prior to re-issuance by another.

Double Selling An instance in which a specific unit of GHG emission reduction or removals is sold to multiple buyers. This can be avoided by having program rules and oversight processes in place to prevent double issuance and double use.

Double Use An instance in which a specific unit of GHG reduction or removal is owned by more than one entity at a given time. Emission Reduction Ton (ERT) The “ERT” is the ACR unit of exchange for tradable, project-based carbon offsets. ERTs refer to both emission reductions and enhancements in sequestration. ACR issues one ERT for each metric tonne of CO2e emission reductions or removals verified against an ACR standard and methodology.

Geologic Sequestration Geologic sequestration is the process of capturing carbon dioxide from a stationary source and injecting it deep underground through a well, with or without enhanced oil recovery. Geologic sequestration is also called carbon capture and storage (CCS).

Greenhouse Gas (GHG) A GHG is any gaseous compound that absorbs infrared radiation in the atmosphere and contributes to the warming of the atmosphere. The primary GHGs regulated under the Kyoto Protocol are carbon dioxide (CO2), nitrous oxide (N20), methane (CH4), hydrofluorocarbons (HFCs), perfluorocarbons (PFCs), and sulfur hexafluoride (SF6). The Intergovernmental Panel on Climate Change lists, and periodically updates, GHGs in its assessment reports. ACR's scope includes all GHGs (including OzoneDepleting Substances) listed in the IPCC Fourth Assessment Report (AR4), Working Group 1, Chapter 2, Table 2.14.26

GHG Emission Reductions and Removals A GHG emission reduction is the measured decrease of GHG emissions over a specified period of time relative to an approved baseline.

A GHG removal is the mass of GHGs removed from the atmosphere over a specified period of time relative to an approved baseline.

GHG Emission System/Trading Program A voluntary or regulated program that allows for trading in project-based GHG emission reductions or removals, government-issued credits, and/or allowances.

GHG Project Plan A GHG Project Plan is a document that describes the Project Activity, satisfies eligibility requirements, identifies sources and sinks of GHG emissions, establishes project boundaries, describes the baseline scenario, defines how GHG quantification will be done and what methodologies, assumptions and data will be used, and provides details on the project's monitoring, reporting and verification procedures. ACR requires every project to submit GHG Project Plan using an ACR-approved methodology.

Global Warming Potential (GWP) Global warming potential is a relative scale translating the global warming impact of any GHG into its CO2 equivalent over the same timeframe. The Intergovernmental Panel on Climate Change periodically updates the list of GHGs and their GWP factors, based on the most recent science. ACR requires Project Proponents to calculate GHG reductions and removals based on the 100-year GWPs in the IPCC Fourth Assessment Report (AR4), Working Group 1, Chapter 2, Table 2.14.

Intergovernmental Panel on Climate Change (IPCC) The IPCC is “the leading body for the assessment of climate change, established by the United Nations Environment Programme (UNEP) and the World Meteorological Organization (WMO) to provide the world with a clear scientific view on the current state of climate change and its potential environmental and socio-economic consequences.”27

Leakage Leakage refers to a decrease in sequestration or increase in emissions outside project boundaries as a result of project implementation. Leakage may be caused by shifting of the activities of people present in the project area, or by market effects whereby emission reductions are countered by emissions created by shifts in supply of and demand for the products and services affected by the project.

Methodology A methodology is a systematic approach that establishes requirements for a Project Proponent to develop the project baseline scenario(s) and to measure, monitor, report and verify emissions reductions or removals by following scientific good practice. Good practice entails that a methodology be conservative, transparent, and thorough.

Methodology Deviations and Revisions A methodology deviation is a project-specific change to an existing approved methodology due to a change in the conditions, circumstances or nature of a project. A deviation may be accepted for a specific project but does not result in an approved modification to the methodology. A methodology revision is a fundamental change in an existing approved methodology due to a change in conditions, circumstances or general developments in knowledge. ACR approval of methodology deviations and modifications is determined by the relevant ACR Technical Committee. Approval of methodology revisions requires public consultation and peer review.

Methodological Tools An approved component of a methodology (i.e., a stand-alone methodological module to perform a specific task) or a calculation tool (i.e., spreadsheets or software that perform calculation tasks) that a Project Proponent uses to quantify net GHG reductions/removals or meet other ACR requirements.

Minimum Project Term The minimum length of time for which a Project Proponent commits to project continuance, monitoring and verification.

Net Emissions Reductions Net Emissions Reductions are GHG emission reductions or removals created by a project activity, minus the baseline scenario and any deductions for leakage.

Ozone-Depleting Substances Ozone-depleting substances (ODS) include controlled substances under Annexes A, B, C and E of the Montreal Protocol.28 Many ODS are also potent GHGs. The Montreal Protocol controls the consumption, production and international trade of ODS, but not emissions, and thus destruction of ODS in already existing facilities and equipment worldwide has the potential to prevent significant GHG emissions.

Permanence GHG removal enhancements may not be permanent if a project has exposure to risk factors, including unintentional reversals unintentional reversals (e.g., fire, flood, insect infestation etc. for terrestrial projects; unanticipated releases of CO2 for geologic projects) and intentional reversals (e.g., landowners or Project Proponents choosing to discontinue project activities).

Permanence Risk Analysis To account for and mitigate against the risk of reversal in some projects, ACR requires Project Proponents to conduct a risk analysis to determine the number of offsets that must be set aside in the ACR buffer pool (unless the Proponent elects a different ACR-approved risk mitigation mechanism). The risk analysis evaluates several types of risk—project, economic, regulatory, and social and environmental/natural disturbance—and must be conducted using an ACR-approved risk analysis/buffer determination tool.

Programmatic Development Approach (PDA) A project in which successive Cohorts of fields, producers or facilities are added incrementally to a project over time. A PDA must be coordinated by a Project Proponent (public or private entity) that must use an approved baseline and monitoring methodology that defines the appropriate boundary, avoids double counting, accounts for leakage, and ensures that the emission reductions are real, measurable, verifiable, and additional to any that would occur in the absence of the project.29

Project Boundaries GHG project boundaries include a project's physical boundary or implementation area, the GHG sources, sinks and reservoirs (or pools) considered, and the project duration.

Project Proponent An individual or entity that undertakes, develops, and/or owns a project. This may include the project investor, designer, and/or owner of the lands/facilities on which project activities are conducted. The Project Proponent and landowner/facility owner may be different entities. The Project Proponent is the ACR account holder.

Registry System An online platform operated by ACR and powered by APX that tracks ownership of offsets, houses a public database of all ACR projects, offset issuances, cancelations and retirements, and provides transparent public access to project documents. https://acr2.apx.com/mymodule/mypage.asp

Reversal An intentional or unintentional event that results in the emissions into the atmosphere of stored or sequestered CO2-e for which carbon offsets (ERTs) were issued.

Standard A standard is an established norm or requirement in a formal document that establishes uniform engineering or technical criteria, methods, processes and practices. Standards may provide general guidance across all project types, such as this document, or be sector-specific. While ACR may accept methodologies and tools from other GHG programs, ACR only registers projects meeting ACR's own standards.

Start Date ACR defines the Start Date for all projects other than AFOLU as the date on which the project began to reduce GHG emissions against its baseline. ACR defines the start date for AFOLU projects as the date on which the Project Proponent began the activity on project lands, with more specific guidance in the relevant ACR sector standards.

Validation Validation is the systematic, independent and documented process for the evaluation of a GHG Project Plan against applicable requirements of the ACR Standard, sector standard and approved methodology.

Validation/Verification Body A competent and independent person, persons or firm responsible for performing the validation and/or verification process. To conduct verification the VVB must be ACR-approved.

Verification Verification is the systematic, independent and documented assessment by a qualified and impartial third party of the GHG assertion for a specific reporting period. The verification process is intended to assess the degree to which a project complies with ACR-approved methodologies, tools, eligibility criteria, requirements, and specifications, and has correctly quantified net GHG reductions or removals. Verification must be conducted by an independent third-party verifier.

Verification Statement A verification statement provides assurance that, through examination of objective evidence by a competent and independent third party, a GHG assertion is in conformity with applicable requirements.

### APPENDIX B: NORMATIVE REFERENCES

The ACR Standard is based on the foundation laid by the normative reference standards and documents listed in Table A-1. These documents assisted ACR to articulate its own requirements and specifications for the quantification, monitoring, and reporting of GHG project-based emissions reductions and removals, verification, project registration, and issuance of project-based offsets.

The ACR Standard builds in particular on the ISO technical specifications for GHG accounting, GHG assertions and verification, and verifier accreditation as set forth in the ISO 14064, Parts 1-3:2006 and ISO 14065:2007 specifications. To the ISO specifications, ACR adds its own mandatory requirements as detailed in the ACR eligibility criteria, additionality determination process, sector standards, and approved methodologies and tools. In the event of conflicts between the ACR Standard and the ISO technical specifications or other normative references, the ACR Standard shall take precedence.

### Table A-1—Normative References for the ACR Standard

### Authoring Body Document or Standard Relationship to ACR

### Clean Development Mechanism (CDM)

□ Project-level baseline and monitoring tools and methodologies □ Tool for the Demonstration and Assessment of Additionality □ GHG sources and sinks significance test

ACR generally accepts approved CDM methodologies for baselines and monitoring. The CDM additionality tool informs ACR additionality tests and may assist Project Proponents in formulating additionality arguments.

### Intergovernmental Panel on Climate Change (IPCC)

□ Guidelines for National GHG Inventories □ Good Practice Guidance □ Fourth Assessment Report

Identification of best practice and options for GHG emission inventory development; methodological guidance and primary seed document for more specific guidance materials and standards

### International Standardization Organization (ISO)

□ ISO 14064:2006, Parts 1-3: a set of international standards that address the quantification, reporting, and verification of GHG emissions and project reductions. □ ISO 14065:2007: verifier accreditation requirements.

ISO 14064:2006 provides a foundation for the ACR Standard by providing technical specifications for GHG accounting and reporting for organizational inventories, projects, and verification assertions. ISO 14065: 2007 specifies requirements for verifier accreditation.

### Authoring Body Document or Standard Relationship to ACR

### USEPA Climate Leaders Program

□ Set of sector-specific and cross sector guidance that addresses quantification, reporting and verification of GHG emissions reductions □ Offset project methodologies for several specific project types

Provides guidance for developing inventory baselines, accounting, and reporting, and Inventory Management Plans. Provides guidance for specific sectors and offset project methodologies; source of ACR approved methodologies, tools and emission factors.

### WRI/WBCSD GHG Protocol

□ GHG Protocol for Project Accounting (2005) □ GHG Protocol for Corporate Inventory Accounting (2005)

Guidance related to additionality—common practice test.

### APPENDIX C: COMPLAINTS & APPEALS PROCEDURE

### A. Complaints Procedure

When a project proponent or ACR stakeholder maintains an objection to a decision made by representatives of ACR or the application of the ACR program requirements, the confidential complaint procedure detailed below shall be followed:

1) Project Proponent or ACR stakeholder sends a written complaint via email to ACR@winrock.org. The complaint must detail the following: □ Description of the complaint with specific reference to ACR Standard and/or ACR methodology requirements, as applicable; □ Supporting documentation provided for consideration by ACR in the complaint resolution process; and □ Complainant name, contact details, and organization.

2) ACR Senior Management shall assign an ACR representative to research and further investigate the complaint. The ACR representative assigned to handle the complaint shall not have been involved previously with the issue that is the subject of the formal complaint.

3) ACR Senior Management will provide a written response, via email, to the complainant detailing ACR's decision on the matter.

### B. Appeals Procedure

In the event that a complaint remains unresolved after the conclusion of the complaints procedure, an ACR project proponent or stakeholder may appeal any such decision or outcome reached as a result of the complaint procedure. The following confidential appeals procedure shall be followed:

Description of the appeal with specific reference to ACR Standard and/or ACR methodology requirements, as applicable; Supporting documentation provided for consideration in the appeal process, including previous communication on the complaint and all relevant details of the previously implemented complaint procedure; and Appellant name, contact details, and organization.

The ACR is also publishing an American Carbon Registry Standard to provide additional guidance as to the ACR operating procedures, as well as other sector-based standards such as the published American Carbon Registry Forest Carbon Project Standard (FCPS). The ACR's intent with these documents is to support the development of the voluntary and pre-compliance U.S. carbon markets. The requirements in ISO 14064, Parts 2-3:2006 and ISO 14065:2007 are the foundation for all of the ACR's offset standards.

American Carbon Registry™ Eligibility Rules and Criteria for GHG Project Registration and Offset Issuance

Greenhouse gas (GHG) reduction and removal projects must meet the American Carbon Registry™ (“ACR”) eligibility criteria below in order to qualify for project registration and GHG offset (carbon offset) issuance.

ACR recommends the adoption of and compliance with Registry methodologies where they exist. ACR does consider baseline, quantification, and monitoring methodologies and tools from other systems including the Clean Development Mechanism (CDM), GHG Protocol, International Standards Organization 14064-2 (ISO), U.S. EPA Climate Leaders and the Voluntary Carbon Standard (VCS) to the extent they conform to the American Carbon Registry Technical Standard. The ACR reserves the right to reject a specific methodology and/or tool.

ACR accepts a variety of project types from locations worldwide and does not issue carbon offsets based on renewable energy credits (RECs) and other indirect emissions reductions or removals from U.S. projects. 1 ACR makes no quality distinction between voluntary and pre-compliance offsets.

Project Document A project document (PD) defines how, what, and when a Project Proponent shall measure, monitor, and report the GHG project in order for an independent third party to verify project outcomes. ACR requires a GHG Project Plan for projects using an ACR methodology and tool, or an ACR approved methodology and tool.

ACR requires a Monitoring, Reporting and Verification (MRV) Project Protocol for projects that fall outside of any ACR sector standard, and whose basis is a site-specific approach to GHG quantification and monitoring.

All PDs shall address all ACR eligibility criteria in this table and include content in accordance with ISO 14064-2:2006, Clause 5.2.

Start Date ACR defines project start date for non-forest/land-use-change projects as the date by which the project began to reduce GHG emissions against the project's baseline.
Non-forest/land use change projects with a start date of 1 Jan. 2000 or later are eligible for ACR registration.

1 ACR registers emissions reductions from projects in the developing world including renewable energy projects 100 MW or less and energy efficiency projects in which the baseline includes indirect emissions. Projects in developing countries require host country approval from the Designated National Authority (DNA) for that country in countries that are signatories to the UN Framework Convention on Climate Change.

ACR defines the start date for forest and land-use-change projects as the date by which the Project Proponent began the project activity on project lands.

Forest and land-use-change projects with a start date of 1 Nov. 1997 or later are eligible for ACR registration.

Forest and land-use-change projects with an earlier start date will be evaluated on a case-by-case basis and accepted or rejected based on the conformity of the methodology to best practices for monitoring carbon storage in forestry and agroforestry projects and conformity with ACR Standards.

Real A real offset yields after-the-fact, quantifiable and verifiable GHG emissions reductions or removals, i.e., after-the-fact atmospheric benefit. ACR requires that the GHG reduction or removal exist prior to offset issuance. ACR will not forward issue nor forward register a projected stream of future offsets.

Additional Additionality is a test to ensure that a project-based offset is “in addition to” reductions and/or removals that would have occurred without carbon market incentives.
ACR requires every project to pass either an approved performance standard and a regulatory additionality test or a three-pronged test of additionality in which the project must: 1) exceed regulatory/legal requirements; 2) go beyond common practice; and 3) overcome one of three implementation barriers: institutional, financial or technical.

Direct Emissions An emission reduction or removal is a “direct emission” if the Project Proponent owns or has control over the source of the emissions (e.g., equipment) or the emissions sink (e.g., project lands). ACR requires a Project Proponent to own or have control over the GHG sources or sinks from which the emissions reduction or removal originates (note exception in footnote 1).

Project Action The project action is the action that results in a GHG removal or reduction.
ACR requires that the project demonstrate an accepted and discernable project action, change in activity or process, and/or avoidance of commonly occurring action.

Title Title is a legal term representing rights and interests in an offset, a future stream of offsets, or a project delivering offsets. ACR requires documentation and attestation of the Project Proponent's undisputed title to all GHG reductions and removals prior to offsets issuance. Title must be clear, unique, and uncontested.

Approved Methods, Tools and Emissions Factors Approved methods and tools include a systematic explanation of how a Project Proponent established the project baseline, estimates and monitors emissions reductions or removals, determines reversal risk and estimates leakage by following scientific good practice. Good practice entails that a Project Proponent be conservative, transparent and thorough. ACR requires use of best practices as incorporated in ACR approved methodologies for baseline determination, baseline update, additionality determination, permanence risk analysis, buffer determination, land eligibility, GHG modeling and measurement, and monitoring and reporting.

Project Baseline The project baseline is a counterfactual scenario that forecasts the likely stream of GHG emissions reductions or removals to occur if the project proponent does not implement the project, i.e., the “business as usual” case.2 Baseline calculations must be consistent with the WRI/WBCSD GHG Protocol and ISO 14064-2, and consider ACR-approved best practices.

Project Proponents shall estimate the baseline for all forest and land use projects at the project start. An approved verifier will verify the baseline at time of offset issuance.

Project Proponents shall use appropriate methodologies and tools to estimate and update forest project baselines.

Permanent Permanence is a reference to the longevity of the atmospheric benefit created by geologic and terrestrial (e.g., carbon that is stored in biomass and soil) sequestration.

Project Proponents must address the risk of reversal by use of one of the following: an approved insurance product to guarantee offsets; a carbon buffer pool; access to a secure source of replacement offsets; or other approved risk management technique.

2 The quantity of offsets that a project generates is the difference between actual emissions or removals and the baseline emissions or removals resulting from the project action.
Fire, disease, pests, tillage and illegal logging can reduce terrestrial carbon stocks and result in the reversal of carbon sequestration, i.e., the atmospheric benefit is not permanent. ACR requires terrestrial sequestration Project Proponents to use the approved “Tool for AFOLU Non-Permanence Risk Analysis and Buffer Determination” in order to address reversal risk and to determine the size of the buffer.

ACR requires geologic sequestration Project Proponents to use approved methodologies that assure permanence including ongoing QA/QC and long-term monitoring.

Net of Leakage Leakage is the increase in GHG emissions outside the project boundary that occurs because of the project action. ACR requires Project Proponents to assess, account for, and mitigate leakage, and provide documentation to support mitigation assertions.

Project Proponents must deduct all leakage that reduces the GHG emissions reduction and/or removal benefit of the project. ACR assesses leakage on a case-by-case basis.

Crediting Period Crediting period is the finite length of time for which the project's PD is valid (i.e., the GHG Project Plan or MRV Project Protocol) and that the ACR can issue offsets for the project. ACR requires a crediting period often (10) years or less for non-forest projects, with opportunities for renewal.

ACR requires afforestation/reforestation (AR) projects to have a crediting period of thirty-five (35) years or less, with opportunities for renewal.

ACR requires improved forest management (IFM) and reduced emissions from deforestation and degradation (REDD) projects to have a crediting period often (10) years or less, with opportunities for renewal.

To renew the crediting period, Project Proponents must secure the services of an independent, approved verifier to validate the PD as using Best Practice methodologies, tools, factors, and monitoring processes at that time.

In the absence of a renewed crediting period, the ACR will cease to issue offsets from that project.

Independent Verification Verification is the independent assessment by a qualified and impartial third party of GHG emissions reduction/removal. The outcome is a verification report and a statement signed by the lead verifier that provides an opinion on the relevance, completeness, accuracy, reliability, and transparency of the GHG quantification data and methods. ACR requires third party verification by an approved verifier prior to offset issuance and as scheduled in the project's PD.

Verifiers must use transparent and replicable verification methods against the relevant ACR standard. ACR reserves the right to reject the verification report from an approved verifier.

Community and Environmental Impacts Projects have the potential to generate both positive and negative community and environmental impacts. Prior to registration, ACR requires all projects to document a mitigation plan for any foreseen negative community or environmental impacts. ACR also requires written disclosure by the Project Proponent of any prior negative environmental or community impacts or claims of negative environmental and community impacts.

ACR requires written disclosure by the Project Proponent of any unmitigated, or claims of unmitigated, negative community and environmental impacts caused by the project as they become known. Furthermore, the Project Proponent must document plans for mitigation of any such reported negative environmental or community impacts.

ACR reserves the right to remove offsets from the Registry on a case-by-case basis.

### American Carbon Registry™ Approach to Additionality

Additionality is a test intended to ensure that project offsets are “in addition to” reductions and/or removals that would have occurred in the absence of the project activity and without carbon market incentives. A project developer/proponent must demonstrate to the American Carbon Registry (“ACR”) that the greenhouse gas (GHG) emissions reductions associated with an offset project are not a “business as usual” baseline scenario.

To qualify as additional, ACR requires every project to pass either an approved performance standard and a regulatory additionality test or a three-pronged test of additionality as described below.

### ACR Hybrid Approach

For projects not using an approved performance standard, the ACR uses a hybrid approach that combines three key tests to determine project additionality. These three tests help the ACR to identify in particular whether realizing a GHG emissions reduction/sequestration goal was a reason, even if only one among many, to implement the project. The three tests are:

Regulatory Surplus Common Practices Implementation Barriers

American Carbon Registry Hybrid Additionality Test

Test

Key Questions

Regulatory Surplus Is there an existing law, regulation, statute, legal ruling, or other regulatory framework in effect now or as of the project start date that mandates the project or effectively requires the GHG emissions reductions?

Yes=Fail; No=Pass

Common Practices In the field or industry/sector, is there widespread deployment of this project, technology, or practice within the relevant geographic area?

Yes=Fail; No=Pass

Implementation Barriers

Financial Choose one (1) of the following three (3):

Does the project face capital constraints that carbon revenues can potentially address; or is carbon funding reasonably expected to incentivize the project's implementation; or are carbon revenues a key element to maintaining the project action's ongoing economic viability after its implementation?

Yes=Pass; No=Fail

Technological

Institutional Is a primary reason for implementation of the technology in question its GHG reduction capabilities or benefits, and is the reduction/sequestration of GHGs one of the goals of the project at the start date?

Yes=Pass; No=Fail

Does this project face significant organizational, cultural, or social barriers to GHG emissions reduction/sequestration that the accrual of benefits from a GHG emissions reduction/sequestration project action will help to overcome?

Yes=Pass; No=Fail

If the project passes the Regulatory Surplus and Common Practices tests, and at least one Implementation Barrier test (i.e., financial, technological, or institutional), ACR considers the project additional.

### Regulatory Surplus Test

The regulatory surplus test involves existing laws, regulations, statutes, legal rulings, or other regulatory frameworks that directly or indirectly affect GHG emissions associated with a project action or its baseline candidates, and which require technical, performance, or management actions. These legal requirements may involve the use of a specific technology, meeting a certain standard of performance, or managing operations according to a certain set of criteria or practices (e.g., forest management practices). The ACR does not consider mandatory those voluntary agreements without an enforcement mechanism, proposed laws or regulations, or general government policies.

### Common Practices Test

Common practices represent the predominant technology(ies) implemented or industry practice(s) undertaken in a particular industry sector and/or geographic region, as determined by the degree to which those technologies/practices have penetrated the market (in a specific geographic area). The proposed offset project must reduce GHG emissions below levels produced by common practices technologies within a comparable environment (e.g., regulatory framework, investment climate, access to technology/financing, etc.).

The level of penetration that represents common practice may differ between sectors and geographic areas, depending on the diversity of baseline candidates. The common practice penetration rate or market share for a technology or practice may be quite low if there are many alternative technologies and practices. Conversely, the common practice penetration rate or market share may be quite high if there are few alternative technologies or practices. Projects that are “first-of-its-kind” are not common practice.

### Implementation Barriers Test

An implementation barrier represents any factor or consideration that would prevent the adoption of such a practice/activity proposed by the project action. Baseline candidates each may face multiple barriers. Generally, there are no barriers to the continuation of current activities, with the exception of regulatory or market changes that force a shift in a project activity, or the end of equipment's useful lifetime.

Under the implementation barriers test, project developers/proponents must choose at least one

(1) among three (3) barrier assessments: i) financial, ii) technological, and iii) institutional. The ACR does not require passing all three (3) barriers. These are:
Financial—Financial barriers can include high costs, limited access to capital, and high risks such as unproven technologies or business models, poor credit rating of project partners, and project failure risk.
Technological—Technological barriers can include R&D deployment risk, uncorrected market failures, lack of trained personnel and supporting infrastructure for technology implementation, and lack of knowledge on practice/activity.
Institutional—Institutional barriers can include institutional opposition to technology implementation, limited capacity for technology implementation, lack of management consensus, aversion to upfront costs, and lack of awareness of benefits.

The current state of carbon credit markets is anyone that wants to buy and sell credits is allowed to do so. What is needed is a carbon credit trading market that is limited to registered producers and consumers that are required to apply for registration and processed through an acceptance criterion before being allowed to participate. This will ensure that only appropriate buyers and sellers participate, and will dramatically reduce potential fraud.

### ACR Validation and Verification Standard

The American Carbon Registry (ACR) requires independent third-party validation and verification of all carbon offset projects following ACR Validation and Verification Guidelines.

Verification is a risk-based process carried out in conformance with ISO 14064-3:2006 and ISO 14065:2007. ACR validation and verification bodies (VVBs) shall be accredited for project validation and verification and the scope of the applicable methodology, and VVB teams shall meet the competence requirements as set out in ISO 14065:2007. VVBs shall also be approved by ACR following the requirements detailed on the ACR webpage for Validation and Verification.

ACR is updating its validation and verification guidelines to the ACR Validation and Verification Standard. Stakeholders are invited to submit comments to ACR@Winrock.org by Apr. 30, 2017.

### Validation and Verification

The American Carbon Registry (ACR) requires independent third-party validation and verification of all carbon offset projects following ACR Validation and Verification Guidelines.

Verification is a risk-based process carried out in conformance with ISO 14064-3:2006 and ISO 14065:2007. ACR validation and verification bodies (VVBs) shall be accredited for project validation and verification and the scope of the applicable methodology, and VVB teams shall meet the competence requirements as set out in ISO 14065:2007.

Verification bodies for California compliance projects must be accredited by the California Air Resources Board (ARB). All VVBs for voluntary market projects must be approved by ACR through the process below, and either be ANSI-accredited in the applicable sectoral scope, or be Designated Operational Entities approved under the Clean Development Mechanism or Accredited Independent Entities approved under Joint Implementation.

### Information Requirements

For voluntary carbon market projects and California Early Action projects, ACR requires that all ACR Approved VVBs execute an Attestation of Validation/Verification Body, which defines the VVB role and responsibilities and ensures technical capabilities and no conflicts of interest. ACR Approved VVBs must also execute a Project-Specific Conflict of Interest Form for each project validated and/or verified.

Validation and Verification activities must address all requirements listed in the American Carbon Registry Standards and follow ACR Validation and Verification Guidelines.

Projects must be verified without reservation, with project proponents having addressed all clarifications and corrections required by the VVB.

Once ACR reviews and accepts a verification statement, and the project proponent has already completed all other required steps, ACR will register the project; post the GHG Project Plan, verification statement, and other documentation to the ACR website; and issue serialized ERTs to the Project Proponent's account.

Validation and Verification Interval ACR requires verification at specified intervals in order to issue new ERTs. Validation of the GHG Project Plan is required once per crediting period. Validation and the first verification may be conducted simultaneously, and may be conducted by the same approved validation and verification body (VVB).

ERTs may be created and issued annually, or at the Proponent's request, more or less frequently. At each request for issuance of new ERTs, the Project Proponent must submit a verification statement from an approved VVB based on a desk audit.

No less than once every five years, Proponents must submit a verification statement based on a full verification including a field visit to the project site. Further qualifications are detailed in the American Carbon Registry Standard v2.1 and relevant sector standards. System Architecture for Utilizing Internet of Things Technology In Conjunction with Energy Management Systems

The purpose of this disclosure is to cover using all aspects of the IoT technological advancements mentioned herein to implement IoT-based energy management systems (EMS). All EMS must fully conform to all the recommended guidelines previously mentioned regarding ISO 14000 certification, including specifically ISO 14064-3 and ISO 14065 in order to be able to act as a carbon credit validator and verifier, which is the primary purpose of the systems being described herein. These energy management systems can take several different forms.

One implementation involves wiring sensors into existing buildings' electrical power supply to record the amount of electricity being used on an ongoing basis. The overall business model is that in the US currently is if an energy efficient system exists that saves more than 25% of the electricity needed to power the system using the old equipment, and the building owner wants it installed in his building, by US Federal requirement, the utility company that supplies power to that building must pay for the equipment and installation of the new energy efficient or water efficient equipment. These efficiency systems can include LED lighting, energy efficient HVAC, heating systems, water aerators, etc. The utility company can either equip sensors to building systems that have recently been upgraded for energy efficiency (see FIG. 3) to measure the power and/or water supply being used, or the Sensor Devices to measure electrical flow and/or water use can be integrated into the efficiency equipment and shipped with the efficiency system being installed. The energy and/or water reduction can then be calculated with the electrical draw and/or water use from the old equipment versus the new, more efficient installations. Electrical flow and/or water use, once measured, can be stored locally and then transmitted to an IoT Platform either through wired and/or wireless network connection. The IoT Edge equipment needed can be integrated into the efficiency system before sending to an installer, or it can be retrofitted to the already installed efficiency system.

Whether equipping the efficiency system with the IoT Edge equipment needed to measure electrical flow and/or water use prior to installation, or after install, both models can support several distinct business models on compensation. Once the carbon credits are generated, validated, verified, and monetized on a trading market or through pre-market contracts, the parties involved can be compensated in a number of overall structures. Either the utility company keeps all the money from the sale of the carbon credits, or it is split between the utility and a third-party ISO 14064 compliant carbon credit validator/verifier. Other business models may include the property owner in any combination with the utility company and/or validator/verifier of the carbon credits generated. The entire mechanism including the monetization of the carbon credits through pre-market, voluntary, regulated/regional markets, and/or cap-and-trade markets, recording of the entire transaction, and payment to individual parties involved can be fully automated, or just parts may be automated for specific purposes.

Another implementation is to retrofit or integrate from factory the Edge hardware needed to measure, monitor and track all electrical flow from renewable resources including solar panels, wind turbines, hydroelectric setups (dams, etc.), biomass, etc. to capture the overall electrical production and transmit it to an IoT Platform via a wireless and/or wired connection. The IoT Edge equipment can be integrated into solar panels, wind turbines, or other renewable resource farm equipment prior to installation of the farm, or after.

Whether equipping a renewable resource farm with the IoT Edge equipment needed to measure electrical flow prior to construction of the farm, or after, both models can support several distinct business models on compensation. Once the carbon credits are generated, validated, verified, and monetized on a trading market or through pre-market contracts, the parties involved can be compensated in a number of overall structures. Either the utility company keeps all the money from the sale of the carbon credits, or it is split between the utility and a third-party ISO 14000 compliant carbon credit validator/verifier. Other business models may include the property owner in any combination with the utility company and/or validator/verifier of the carbon credits generated. The entire mechanism including the monetization of the carbon credits through pre-market, voluntary, regulated/regional markets, and/or cap-and-trade markets, recording of the entire transaction, and payment to individual parties involved can be fully automated, or just parts may be automated for specific purposes.

The same setups can use Edge Gateway and/or Routers with electrical flow measurement capability as well. Any of the Sensor Device/Gateway/Router combinations can then transmit over wireless or wireline network to an IoT Platform implementation. This may involve using sensors built into the efficiency or renewable energy to measure the overall electrical use, and then send that information to an IoT Platform for processing. Once data is transmitted to the IoT Platform, it must implement machine learning and AI techniques, along with any math needed to accurately calculate carbon credits. The IoT Platform may just be a server environment hosting ISO 14064 certified software that can calculate and generate the carbon credits. These calculations may incorporate current weather conditions in for accuracy, and may use weather forecasting models in the calculations to accommodate ISO standards for long term carbon credit projection and calculation.

Another implementation to generate carbon credits could be to measure the electrical production by the braking system of new electric and/or hybrid vehicles. In new Tesla automobiles, for instance, the braking system is designed to capture energy from the movement of the vehicle by generating electricity from the braking system once applied during operation. This generated electricity is fed back into the battery supply for the vehicle and used to further power the main electrical engine for the vehicle, thus extending the distance the vehicle can travel on a single full charge. The energy produced by the braking system can be measured in an ISO 14064-3 compliant verification and/or validation system as specified herein and then the calculated carbon credits can be monetized on a carbon credit trading market or used by the manufacturer to offset their own emissions.

The IoT hardware in use to collect the electrical flow measurements will probably need to support the following wireless protocols: LoRa, WiFi, Zigbee, RSS, Cellular, Bluetooth, OpenWare, etc., as well as the wired protocols: BACnet, Modbus, CT, Temperature, Micro Switches, etc. The equipment should more than likely have batter backup of at least 4 hours, along with a processor, memory, and 1 Tb of storage. The equipment should also support 3-24V power supplies along with including a 24V transformer, as well as must communicate with the Internet via a landline, possibly a telco phone line.

Any combination of sensors and/or Sensor Devices and/or Edge Routers and/or Edge Gateway devices mentioned herein, or any variants, can be used to measure electrical flow and/or water use for purposes of generating carbon credits within an EMS system consistent with the guidelines specified in this disclosure.

Once the carbon credits are generated, validated and/or verified per ISO requirements, they can then be sold to companies to offset their carbon use. This can be done pre-market, or on a voluntary or cap-and-trade/regional carbon market.

The wireless and/or wired communications transport layer and security are considered separate items with TCP/IP (current Internet). They should be merged so that PKI is implemented in the communications protocol itself. It would also be preferred to have a notion of a Local Area Network (LAN) and wide area network at the protocol level on the Edge. ie: hospitals could have local access for individual facilities and manufacturing plants, etc. while the connection to the outside IoT Platform could be controlled over a partitioned Wide Area Network (WAN).

For the IoT Platform storage, a Blockchain storage mechanism (discussed in detail later in this document) can be implemented on one cloud implementation, or implemented across two or more cloud networks in real-time so that each cloud installation mirrors the other cloud installation(s) in real-time. The latter model will improve availability, uptime and overall system security, reliability, stability and integrity.

Resource usage sensors (measuring electricity, gas and water usage) should be intelligent in that they can calculate the overall usage and send out the measurement on a timed interval as opposed to just streaming measurements in real-time to a data storage facility for calculation of usage.

Maintain and document program for calibrating monitoring equipment remotely. All records, certifications, validation and verification reports should be stored in an immutable data storage facility like a Blockchain implementation.

Unlike most other IoT systems, this IoT system should not simply stream data from the sensors to the cloud on an ongoing basis. This system should have enough logic to calculate ongoing usage of electrical power or other sensing information and only send the exception to given rules or an overall count or total of usage on a given time interval, which could be a certain predefined number of minutes, hours, days, monthly or any other appropriate time-based cycle. This will ensure the overall system is only transmitting and/or storing relevant changes in the information being sensed from the sensor devices to the online storage facility, and not additional information that may not be relevant.

The Process to set up users on an energy efficiency program to calculate carbon credits can work in the following manner
Provide at least one year's gas, electric and/or renewable energy production and/or usage statements.
As appropriate, evaluate how to best get you and/or your customers into a custom Utility Energy Efficiency Rebate Program; this will help drive your overall savings.
Perform detailed measurements and modeling to determine estimated costs to build and install a carbon credit generation and monetization system.
Once your Energy Production and/or custom Utility Energy Efficiency Applications are approved and a utility rebate amount is confirmed, we will order the equipment and will install the carbon credit generation and monetization system.
After the installation is complete, final installation inspections are scheduled for the Renewable Energy Producer and/or Energy Utility's approval. * Monetization on a monthly basis occurs and rebates or payments are made to all benefitted parties per contract terms.

### Carbon Credit Trading Platform Design

### What is the Carbon Market?

The new low-carbon economy provides an emerging market opportunity for foresters, ranchers, farmers, electric utilities, building owners, and renewable resource farm (solar, wind, biomass, hydro, etc.) owners to develop and sell carbon credits, also called greenhouse gas offsets and reduction credits. Carbon credits are developed from land conservation and renewable energy projects which reduce the amount of carbon dioxide (CO2)—or “greenhouse gas”—released into the air or remove existing CO2 from the air. Projects often include the use of terrestrial sequestration. Greenhouse gas (GHG) management and carbon credit projects are good for the environment but they also provide an economic opportunity for those who develop them. Investors, often large companies or industries, purchase carbon credits to offset their own CO2 emissions.

Many rural areas contain large land holdings, much of which are currently used for farming, ranching, or forestry. This situation puts Indian nations and landowners in a unique position to derive income from the sale of carbon credits which are based on carbon storage value and require large areas of land in order to be profitable for the project developer. Benefits to carbon market enrollment include:
Additional profit from land
Preservation of Indian land ownership
Promotion of land stewardship
Greenhouse gas emissions reductions
Promotion of soil health, ecological diversity, and water and air quality

In addition, utility companies, renewable resource farms, and/or building owners can also equip additional IoT equipment to monitor energy production and efficiencies to produce and benefit from the sale of carbon credits.

### Carbon Credit Trading and Market Infrastructure

Carbon credits in North America are currently traded on voluntary offset markets and through regional GHG compliance programs. Prices for carbon credits are higher in compliance programs because there is greater demand for credits from regulated entities.

### What is Being Traded?

### Carbon Offsets

The term “carbon offset” is used generically to refer to a ton of carbon dioxide equivalent (CO2e). An offset negates the effects of carbon emitted in one place by avoiding the release of a ton of carbon elsewhere or absorbing/sequestering a ton of CO2e that would have otherwise remained in the atmosphere.

### Allowances

Under regulatory compliance programs, emitters are allocated a specified number of allowances, representing tons of CO2e they may legally emit. An entity that can reduce its annual emissions below the number of allowances received may bank the credits for future compliance or sell the credits/allowances to other entities whose emissions exceed annual allowances. Allowances may include emission reductions or offsets and are generally defined as acceptable emission units recognized by a registry.

### Emission Reductions

Emission reductions are the quantifiable reduction in emissions attributable to an activity or technology.

### Market Participants

Carbon market participants include project sponsors, project developers, aggregators, brokers, verifiers, and buyers.

Project sponsors: The owners of land or a business that undertake an activity or adopt a practice that sequesters carbon or reduces emissions.

Project developer: Responsible for all aspects of the delivery of the carbon offset, including the development of project methodologies, baseline determinations, additionality analysis, and monitoring plans.

Aggregators: May share similar functions as the project developer while also bringing together smaller projects in marketable volumes to buyers, brokers, or exchanges.

Brokers: Project developers (also known as offset providers) may decide to enlist the services of a broker to market offsets and to act as an intermediary with potential buyers. Brokers sort through potential investment opportunities for buyers and create portfolios scalable for large investor demand.

Verifiers: An integral process in the establishment of carbon offset quality and project integrity is independent verification of project offsets by a third-party agent. Verifiers may conduct field based carbon measurements or perform remote audits of entity reports, verifying that registry or standard measurement protocols have been followed during the development of the project and implementation of monitoring, mitigation, and verification.

Buyers: Buyers in the voluntary market fall into three primary categories: retail, industrial, and investment. Ultimately, the majority of transacted offsets are reported to a GHG registry or exchange, where they are retired to mitigate an entity's GHG emissions.

### Voluntary Markets

A voluntary offset market refers to the voluntary sales and purchases of carbon credits where transactions are not part of a GHG compliance “cap-and-trade” program. Voluntary markets are also referred to as the “over-the-counter” market where buyers and sellers engage directly, through a broker. Credits in this market are voluntary emissions reductions, VERs or carbon offsets. Buyer motivations include wanting to manage their climate change impacts, an interest in innovative philanthropy, public relations benefits, the need to prepare for (or deter) federal regulations, and plans to re-sell credits for a profit.

### Certification Programs and Standards

Carbon credits developed from carbon storage projects and emission reduction activities must meet voluntary market standards in order to provide quality assurance for purchasers. Landowners and developers enroll their projects into certification programs which provide GHG accounting protocols for the quantification, monitoring, and reporting of the amount of stored carbon or reduced emissions. Various protocols have been developed and differ across organizations depending on the type of project.

### Compliance Programs

GHG compliance “cap-and-trade” programs place an overall limit on emissions allowed from a specified set of entities, and issue tradable emission allowances (or rights to emit) that these entities can use for compliance. Allowances, which typically authorize an entity to emit a ton of CO2e, can be auctioned or freely distributed to covered entities or other parties. At the end of a program's compliance period, covered entities must submit an allowance for every ton of CO2e they emitted during that period. Every greenhouse cap-and-trade program established to date has also allowed covered entities to submit offsets in lieu of allowances for compliance purposes. A covered entity in a cap-and-trade program, therefore, has several options for achieving compliance: using emission allowances it has received or purchased, acquiring offsets, and/or reducing its own emissions. The following table lays out the cap-and-trade systems currently used throughout the United States.

Beginning in 2013, the state of California will also regulate GHGs through a cap-and-trade compliance program. This program will allow regulated emitters to purchase offsets that meet California Air Resources Board standards and protocols. The currently approved protocols, posted at http://www.arb.ca.gov/cc/capandtrade/offsets/offsets.htm, are for livestock manure biodigesters, ozone depleting substances, urban forest projects, and U.S. forest projects (reforestation, improved forest management, and avoided forest conversion). The Air Resources Board is also evaluating additional protocols for adoption. Projects developed under ARB protocols may be listed on any offset project registry. The American Carbon Registry and the Climate Action Reserve are under consideration by ARB as offset project registries. If the program is determined to be successful it will most likely serve as a model for future market developments.

### Greenhouse Gas Registry

A greenhouse gas registry is an official repository to which an entity reports emissions of one or more GHGs or changes in emission levels, typically annually. Participants can include companies reporting entity-wide or on a project-by-project basis; all or parts of state government operations; individuals; or other parties responsible for emissions or emission reductions. A GHG registry is subject to reporting and verification requirements to ensure data consistency and quality, and registries can support voluntary or mandatory reporting requirements. Aggregators track and report contracted offsets for the purposes of verification.

### Overview of a Blockchain-Based Carbon Credit Trading Platform

The overall trading system technical architecture should implement a “blockchain” based transaction recording mechanism to reduce fraud and improve system reliability.

According to Wiki: A blockchain—originally block chain—is a continuously growing list of records, called blocks, which are linked and secured using cryptography. Each block typically contains a hash pointer as a link to a previous block, a timestamp and transaction data. By design, blockchains are inherently resistant to modification of the data. A blockchain can serve as “an open, distributed ledger that can record transactions between two parties efficiently and in a verifiable and permanent way.” For use as a distributed ledger, a blockchain is typically managed by a peer-to-peer network collectively adhering to a protocol for validating new blocks. Once recorded, the data in any given block cannot be altered retroactively without the alteration of all subsequent blocks, which needs a collusion of the network majority.

Blockchains are secure by design and are an example of a distributed computing system with high Byzantine fault tolerance. Decentralized consensus has therefore been achieved with a blockchain. This makes blockchains potentially suitable for the recording of events, medical records, and other records management activities, such as identity management, transaction processing, documenting provenance, or food traceability.

The first work on a cryptographically secured chain of blocks was described in 1991 by Stuart Haber and W. Scott Stornetta. In 1992, Bayer, Haber and Stornetta incorporated Merkle trees to the blockchain as an efficiency improvement to be able to collect several documents into one block.

The first distributed blockchain was then conceptualized by an anonymous person or group known as Satoshi Nakamoto in 2008 and implemented the following year as a core component of the digital currency bitcoin, where it serves as the public ledger for all transactions. Through the use of a peer-to-peer network and a distributed timestamping server, a blockchain database is managed autonomously. The use of the blockchain for bitcoin made it the first digital currency to solve the double spending problem without requiring a trusted administrator. The bitcoin design has been the inspiration for other applications.

The words block and chain were used separately in Satoshi Nakamoto's original paper in October 2008, and when the term moved into wider use it was originally block chain, before becoming a single word, blockchain, by 2016. In August 2014, the bitcoin blockchain file size reached 20 gigabytes. In January 2015, the size had grown to almost 30 gigabytes, and from January 2016 to January 2017, the bitcoin blockchain grew from 50 gigabytes to 100 gigabytes in size.

By 2014, “Blockchain 2.0” was a term referring to new applications of the distributed blockchain database. The Economist described one implementation of this second-generation programmable blockchain as coming with “a programming language that allows users to write more sophisticated smart contracts, thus creating invoices that pay themselves when a shipment arrives or share certificates which automatically send their owners dividends if profits reach a certain level.” Blockchain 2.0 technologies go beyond transactions and enable “exchange of value without powerful intermediaries acting as arbiters of money and information”. They are expected to enable excluded people to enter the global economy, enable the protection of privacy and people to “monetize their own information”, and provide the capability to ensure creators are compensated for their intellectual property. Second-generation blockchain technology makes it possible to store an individual's “persistent digital ID and persona” and are providing an avenue to help solve the problem of social inequality by “[potentially changing] the way wealth is distributed”. As of 2016, Blockchain 2.0 implementations continue to require an off-chain oracle to access any “external data or events based on time or market conditions [that need] to interact with the blockchain”.

In 2016, the central securities depository of the Russian Federation (NSD) announced a pilot project based on the Nxt Blockchain 2.0 platform that would explore the use of blockchain-based automated voting systems. Various regulatory bodies in the music industry have started testing models that use blockchain technology for royalty collection and management of copyrights around the world. [better source needed] IBM opened a blockchain innovation research centre in Singapore in July 2016. A working group for the World Economic Forum met in November 2016 to discuss the development of governance models related to blockchain. According to Accenture, an application of the diffusion of innovations theory suggests that in 2016 blockchains attained a 13.5% adoption rate within financial services, therefore reaching the early adopters phase. In 2016, industry trade groups joined to create the Global Blockchain Forum, an initiative of the Chamber of Digital Commerce.

In early 2017, the Harvard Business Review suggested that blockchain is a foundational technology and thus “has the potential to create new foundations for our economic and social systems.” It further observed that while foundational innovations can have enormous impact, “It will take decades for blockchain to seep into our economic and social infrastructure.”

A blockchain facilitates secure online transactions. A blockchain is a decentralized and distributed digital ledger that is used to record transactions across many computers so that the record cannot be altered retroactively without the alteration of all subsequent blocks and the collusion of the network. This allows the participants to verify and audit transactions inexpensively. They are authenticated by mass collaboration powered by collective self-interests. The result is a robust workflow where participants' uncertainty regarding data security is marginal. The use of a blockchain removes the characteristic of infinite reproducibility from a digital asset. It confirms that each unit of value was transferred only once, solving the long-standing problem of double spending. Blockchains have been described as a value-exchange protocol. This blockchain-based exchange of value can be completed more quickly, more safely and more cheaply than with traditional systems. A blockchain can assign title rights because it provides a record that compels offer and acceptance.

A blockchain database consists of two kinds of records: transactions and blocks. Blocks hold batches of valid transactions that are hashed and encoded into a Merkle tree. Each block includes the hash of the prior block in the blockchain, linking the two. Variants of this format were used previously, for example in Git. The format is not by itself sufficient to qualify as a blockchain. The linked blocks form a chain. This iterative process confirms the integrity of the previous block, all the way back to the original genesis block. Some blockchains create a new block as frequently as every five seconds. As blockchains age they are said to grow in height.

Sometimes separate blocks can be produced concurrently, creating a temporary fork. In addition to a secure hash based history, any blockchain has a specified algorithm for scoring different versions of the history so that one with a higher value can be selected over others. Blocks not selected for inclusion in the chain are called orphan blocks. Peers supporting the database have different versions of the history from time to time. They only keep the highest scoring version of the database known to them. Whenever a peer receives a higher scoring version (usually the old version with a single new block added) they extend or overwrite their own database and retransmit the improvement to their peers. There is never an absolute guarantee that any particular entry will remain in the best version of the history forever. Because blockchains are typically built to add the score of new blocks onto old blocks and because there are incentives to work only on extending with new blocks rather than overwriting old blocks, the probability of an entry becoming superseded goes down exponentially as more blocks are built on top of it, eventually becoming very low. For example, in a blockchain using the proof-of-work system, the chain with the most cumulative proof-of-work is always considered the valid one by the network. There are a number of methods that can be used to demonstrate a sufficient level of computation. Within a blockchain the computation is carried out redundantly rather than in the traditional segregated and parallel manner.

By storing data across its network, the blockchain eliminates the risks that come with data being held centrally. The decentralized blockchain may use ad-hoc message passing and distributed networking. Its network lacks centralized points of vulnerability that computer crackers can exploit; likewise, it has no central point of failure. Blockchain security methods include the use of public-key cryptography. A public key (a long, random-looking string of numbers) is an address on the blockchain. Value tokens sent across the network are recorded as belonging to that address. A private key is like a password that gives its owner access to their digital assets or otherwise interact with the various capabilities that blockchains now support. Data stored on the blockchain is generally considered incorruptible.

Every node or miner in a decentralized system has a copy of the blockchain. Data quality is maintained by massive database replication and computational trust. No centralized “official” copy exists and no user is “trusted” more than any other. Transactions are broadcast to the network using software. Messages are delivered on a best effort basis. Mining nodes validate transactions, add them to the block they are building, and then broadcast the completed block to other nodes. Blockchains use various time-stamping schemes, such as proof-of-work, to serialize changes. Alternate consensus methods include proof-of-stake and proof-of-burn. Growth of a decentralized blockchain is accompanied by the risk of node centralization because computer resources required to operate bigger data become more expensive.

The blockchain mechanism could be used for registering users of the IoT implementation, as well as registering all the equipment necessary to implement the carbon credit generation and monitoring software platform, potentially in a Cloud-computer based environment. One could foresee the blockchain implementation within a single Cloud-computing environment, or spanning across two or more Cloud-computing environments. If the blockchain implementation was spread across multiple Clouds, this would increase security as well as availability and stability of the entire system. All transactions could be recorded by the blockchain so that the entire IoT implementation benefits from the blockchain's benefits.

### Carbon Credit Trading

The carbon credit trading market should support the following types of general trading transactions, known as market orders. What is a market order? A market order instructs a broker to buy or sell securities for your account at the next available price. It remains in effect only for the day, and usually results in the prompt purchase or sale of all the shares of carbon credits, options or contracts (potentially pre-market contracts that represent a purchase of carbon credits) as long as the security is actively traded and market conditions permit. Note: In order to maintain a fair and orderly market, most market centers generally do not accept cancellation requests after 9:28 a.m. ET for market orders eligible for execution at 9:30 a.m. ET, when the market opens. Acceptance of a cancellation request by a broker between 9:28 and 9:30 a.m. ET does not guarantee an order cancellation. All requests to cancel an order are processed on a best-efforts basis.

What is a limit order? When you place a limit order to buy, the carbon credit is eligible to be purchased at or below your limit price, but never above it. You may place limit orders either for the day on which they are entered (a day order), or for a period that ends when it is executed or when you cancel (an open order or good 'til canceled (GTC) order). Note: All open GTC orders will expire 180 calendar days after they are placed. If the 180th day falls on a weekend or holiday, those orders will expire on the first business day following the expiration day. This policy does not apply to options.

Orders at each price level are filled in a sequence that is determined by the rules of the various market centers; therefore, there can be no assurance that all orders at a particular price limit (including yours) will be filled when that price is reached. Such orders are also subject to the existence of a market for that security. Thus, the fact that your price limit was reached does not guarantee an execution.

Limit orders for more than 100 shares or for multiple round lots (200, 300, 400, etc.) may be filled completely or in part until completed. It may take more than one trading day to completely fill a multiple round lot or mixed-lot order unless the order is designated as one of the following types:

All or none (fill the whole order or no part of it). When you place an all-or-none designation on your order, it is considered restricted. The carbon credit can trade at or below your price on a buy, or at or above on a sell, without the right to execution, unless the entire amount of your order is executable.

Immediate or cancel (fill the whole order or any part immediately, and cancel any unfilled balance).
Fill or kill (fill the entire order immediately or cancel it).

Note: Good 'til canceled time in force is not available for short sales, unlisted corporate bonds and treasuries, mortgage-backed and agency bonds, and collateralized mortgage obligations (CMOs). We do not accept limit orders for municipal bonds, commercial paper, unit investment trusts (UITs), certificates of deposit (CDs), or mutual funds.

### What is a Stop Order?

Stop orders are generally used to protect a profit or to prevent further loss if the price of a security moves against you. They can also be used to establish a position in a security if it reaches a certain price threshold or to close a short position.

The specialists on the various exchanges and market makers have the right to refuse stop orders under certain market conditions. Not all securities or trading sessions (pre- and post-market) are eligible for stop orders.

### Types of Stop Orders

### Stop Loss

This type of order automatically becomes a market order when the stop price is reached. Therefore, there is no guarantee that your order will be executed at the stop price.

It is important for investors to understand that company news or market conditions can have a significant impact on the price of a security. This could result in a stop loss order being executed at a price that is dramatically different than what your stop loss price indicates.

### Stop Limit

This type of order automatically becomes a limit order when the stop price is reached. Like any limit order, a stop limit order may be filled in whole, in part, or not at all, depending on the number of shares available for sale or purchase at the time.

Note: Buy stop loss and buy stop limit orders must be entered at a price which is above the current market price. Sell stop loss and sell stop limit orders must be entered at a price which is below the current market price.

### How Stop Orders are Triggered

### Carbon Credits

Equity stop orders placed with a broker are triggered off of a round lot transaction of 100 shares or greater, or a print in the security. The market centers to which National Financial Services (NFS) routes a broker's stop loss orders and stop limit orders may impose price limits such as price bands around the National Best Bid or Offer (NBBO) in order to prevent stop loss orders and stop limit orders from being triggered by potentially erroneous trades. These price limits may vary by market center. If you are interested in placing an order which triggers off of a bid quote or ask quote, please see Trailing Stop Orders and Contingent Orders.

### Options

Generally a stop order to buy becomes a market order when the bid price is at or above the stop price, or the option trades at or above the stop price. A stop order to sell becomes a market order when the ask price is at or below the stop price, or when the option trades at or below the stop price. The options stop election is based on the exchange's best bid or offer (BBO) where the stop order resides.

### What Time Limitations can I Place on an Order?

You place a time limitation on a carbon credit trade order by selecting one of the following time-in-force types:

### Day

A time-in-force limitation on the execution of an order. This limitation has a default order expiration time of 4:00 p.m. ET. You may select your own order expiration time between 10:00 a.m. ET and 4:00 p.m. ET in thirty-minute increments (i.e., 10:00 a.m., 10:30 a.m., 11:00 a.m., etc). If all or part of your order is not executed by the time you've selected for expiration, your order will be canceled. You may view the status of your order, including order expiration date and/or time, on the Orders page.

### Good 'Til Canceled

A time-in-force limitation that can be placed on a carbon credit or ETF order. This limitation has a default order expiration date of 180 calendar days from the order entry date at 4:00 p.m. ET. You may select your own order expiration date and/or time, up to 180 calendar days from the order entry date. If all or part of your order is not executed by the date and/or time you've selected for expiration, any open portions of your order will be canceled. You may view the status of your order, including order expiration date and/or time on the Orders page.
   Fill or kill
A time-in-force limitation that can be placed on the execution of an order. This limitation requires that the order is immediately completed in its entirety or canceled.

Orders with the fill or kill limitation:
are for 100 shares or more
are only placed during market hours
are good only for the current day
are not allowed for use with stop loss, stop limit, or sell short orders

Note: Fill or kill is only used under very special circumstances. If you do not fully understand how to use fill or kill, talk to a broker before placing this limitation of an order.

### Immediate or Cancel

A time-in-force limitation that can be placed on the execution of an order. This limitation requires that a broker immediately enter a bid or offer at a limit price you specify. All or a portion of the order can be executed. Any portion of the order not immediately completed is canceled.

### On the Open

A time-in-force limitation that can be placed on an order. This limitation requires that the order is executed as close as possible to the opening price for a security. All or any part of the order that cannot be executed at the opening price is canceled.

### On the Close

A time-in-force limitation that can be placed on the execution of an order. This limitation requires that the order is executed as close as possible to the closing price for a security. All or any part of the order that cannot be executed at the closing price is canceled.

### How are Commissions Assessed for Good 'Til Canceled Orders?

The commission for a good 'til canceled order is assessed at the time your order is executed.
If your order receives multiple executions on a single day, you will be assessed one commission. For good 'til canceled orders that receive executions over multiple days, a commission is assessed for each day in which there is an execution.

Good 'til canceled orders that do not execute are not charged a commission.

### How do Dividend Distributions Affect Open Orders?

Although different exchange rules may exist for adjusting orders when a security pays a dividend, the general rule is that good 'til canceled (GTC) orders below the market are adjusted for the dividend amount. The price of your order will be automatically reduced on the “ex-dividend” date by approximately the amount of the upcoming dividend unless you note it as a do not reduce (DNR) when you place the order. Orders below the market include: buy limit, sell stop loss, sell stop limit, sell trailing stop loss, sell trailing stop limit.

### What are the Risks of Trading in Volatile Markets?

Volatile markets can present higher trading risks, especially when you are using electronic services to access information or place orders. Consider placing limit orders instead of market orders. In certain market conditions, or with certain types of securities offerings (in this implementation, carbon credits), price changes may be significant and rapid during regular or after-hours trading. In these cases, placing a market order could result in a transaction that exceeds your available funds, meaning that a broker would have the right to sell other assets in your account to cover any outstanding debt. This is a particular risk in accounts that you cannot easily add money to, such as retirement accounts.

Be aware that quotes, order executions, and execution reports could be delayed. During periods of heavy trading or volatility, quotes that are provided as “real time” may be stale—even if they appear not to be—and you may not receive every quote update. Security prices can change dramatically during such delays. When canceling an order, be sure your original order is actually canceled (verified canceled order status) before entering a replacement order. Don't rely on a receipt for your cancellation order; that order may have arrived too late for us to act on. Cancellation requests are handled on a best-efforts basis. Use other ways to access a broker during peak volume times. System availability and response time may be subject to market conditions. If you are having problems reaching us one way, try another. There are several ways to contact a broker.

The chances of encountering these risks are higher for individuals using day trading strategies. In part for this reason, a broker does not promote day trading strategies. For more information on trading risks and how to manage them, contact a broker.

### Advanced Order Types

### What is a Trailing Stop Order?

Trailing Stop Orders adjust automatically when market conditions move in your favor, and can help protect profits while providing downside protection. With a Trailing Stop Order, you do not have to constantly adjust for price changes.

Additionally, Trailing Stop Orders may have increased risks due to their reliance on trigger processing, market data, and other internal and external system factors. These orders are held in a separate order file with a broker and are not sent to the marketplace until the order conditions you've defined have been met.

### Eligible Securities:

Listed and OTC Equities
Single-leg Options

### Types of Trailing Stop Orders:

Trailing Stop Loss: Once triggered, the order will become a market order.
Trailing Stop Limit: Once triggered, the order will become a limit order.

### Trailing Stop Order Trigger Values:

You may elect to trigger a Trailing Stop order based on the following security market activities:
The security's last round lot trade of 100 shares or greater (default)
The security's bid price
The security's ask price

### Trailing Stop Order Time Limits:

Trailing Stop orders can be either Day orders or Good 'til Canceled (GTC) orders.
GTC orders placed on a broker expire after 180 days.

### Trailing Stop Order Trail Values:

Equity Trailing Stop orders can be set with a percentage (%) or dollar ($) trail value.
Single-leg Option Trailing Stop orders can only be set with a dollar ($) trail value.
   Important information regarding Trailing Stop Orders.

Example of a Trailing Stop Order
trailing_stop
1. You buy carbon credits at $25 per share.
2. carbon credits rises to $27.
3. You place a sell trailing stop loss with a trail value of $1.
4. As long as the price moves in your favor, your trailing price will stay $1 away.
5. The price of carbon credits peaks at $29, then starts to drop. Your trailing stop loss remains at $28.
6. Shares are sold when carbon credits reaches $28.

### What is a Conditional Order?

A conditional order allows you to set order triggers for carbon credits and options based on the price movement of carbon credits, indices, or options contracts. There are five types: Contingent, Multi-Contingent, One-Triggers-the-Other (OTO), One-Cancels-the-Other (OCO), and One-Triggers-a-One-Cancels-the-Other (OTOCO).

### Contingent

A Contingent order triggers an equity or options order based on any 1 of 8 trigger values for any carbon credits, or any valid options contract.
   Trigger values: last trade, bid, ask, volume, change % up, change % down, 52-week high, and 52-week low
   Market, limit, stop loss, and trailing stop loss are available order types once the contingent criterion is met.
   Security type: carbon credits or single-leg options

Time-in-force: For the contingent criteria and for the triggered order, it can be for the day, or good 'til canceled (GTC). The time-in-force for the contingent criteria does not need to be the same as the time-in-force for the triggered order.

### Example of a Contingent Order

contingent
1. You place a Contingent order to buy carbon credits at a limit of $25—if the UVW index moves up more than 1.25%.
2. A rally occurs that pushes the index up 1.30% on the day . . . .
3 . . . which triggers a limit order to buy carbon credits at $25.
4. carbon credits hits your limit of $25 so shares are bought.

Multi-Contingent
A Multi-Contingent order triggers an equity or option order based on a combination of 2 trigger values for any carbon credits. The criteria can be linked by “And at the same time,” “Or,” or “Then.”
“And at the same time” is chosen if both criteria must be met at the same time.
“Or” is chosen if either one of the two criteria must be met.
“Then” is chosen if the criteria must be met in sequential order.
   Trigger values: last trade, bid, ask, volume, change % up, change % down, 52-week high, and 52-week low
   Security type: carbon credits or single-leg options

Time-in-force: For the contingent criteria and for the triggered order, it can be for the day, or good 'til canceled (GTC). The time-in-force for the contingent criteria does not need to be the same as the time-in-force for the triggered order.

### Example of a Multi-Contingent Order

multi_contingent
1. You purchase carbon credits at $25 and place a Multi-Contingent order to sell carbon credits at the market if . . . .
2A. . . . carbon credits last trade is less than $20 . . . .
2B. . . . or carbon credits hits a new 52-week high of $32.
3. carbon credits moves up to $32 which triggers your order to sell. You order fills at $32.01.

One-Triggers-the-Other (OTO)
A One-Triggers-the-Other order actually creates both a primary and a secondary order. If the primary order executes, the secondary order automatically triggers.
This type of order can help you save time: place a buy order as your primary order and a corresponding sell limit, sell stop, or sell trailing stop at the same time. Or, if you trade options regularly, use an OTO order to leg into a buy-write or covered-call position.
Trailing stop orders are available for either or both legs of the OTO.
   Security type: Any combination of carbon credits and/or single-leg options
   Time-in-force: Can be different for each order
   For OTO orders that are good 'til canceled (GTC), the whole order is good for 180 days (e.g., if the primary order executes on day 30, the secondary order is live for 150 days).
   If the primary order is canceled, the secondary order is also canceled.
   If the secondary order is canceled, the primary order remains open as a separate order,

### Example of One-Triggers-the-Other Order

one_triggers
1. You place an OTO to buy carbon credits at $30 and sell at a $2 trailing stop loss.
2. The carbon credits drops to $30, which triggers a buy order of carbon credits that executes and . . . .
3 . . . a sell trailing stop loss order with a $2 trail is placed with an initial trigger price of $28.
4. carbon credits moves up to $35 . . . .
5 . . . so the new trigger price for the trailing stop order is $33.
6. carbon credits trades down to $33, which triggers the trailing stop order and shares are sold at the market.
   One-Cancels-the-Other (OCO)
   With a One-Cancels-the-Other (OCO) order, two orders are live so that if either executes, the other is automatically triggered to cancel.
   When orders are placed for retirement accounts, a price-reasonability check helps prevent both OCO orders from executing in a fast market. This feature does not exist in nonretirement accounts.
      Security type: Any combination of carbon credits or single-leg options
      Time-in-force: Must be the same for both orders
      Orders can be for the same shares of the same carbon credits or option contracts, but on opposite sides of the market (sell limit and sell stop).

### Example of One-Cancels-the-Other Order

one_cancels
1. You buy carbon credits at $23.
2. carbon credits rises to $25.
3. You place an OCO with a sell order of $27 and . . . .
4 . . . a sell stop at $24.
5. carbon credit hits $27, so your sell order executes and . . . .
6 . . . your sell stop order is canceled.
   One-Triggers-a-One-Cancels-the-Other (OTOCO)
   With a One-Triggers-a-One-Cancels-the-Other order, you place a primary order which, if executed, triggers two secondary orders.
   If either of these secondary orders executes, the other is automatically canceled.
      Security type: Any combination of carbon credits or single-leg options
      Time-in-force: Primary can be different than both secondary orders. However, both secondary orders must have the same time-in-force.

### Example of One-Triggers-a-One-Cancels-the-Other Order

One-Triggers-Cancel-Other
1. You place an order to buy carbon credits at $25.
2. At the same time, you place two sell orders, one at stop loss for $23 and one at a limit of $27.
3. carbon credits trades at $25.
4. Your buy order executes.
5. Simultaneously, your two sell orders are triggered.
6. carbon credits drops to $23.
7. Your stop loss order executes and your limit order is automatically canceled.

### What is Short Selling?

Short selling is an advanced trading technique that allows you to integrate a number of different strategies into your overall investment approach so that you may potentially profit from downward moves in a particular carbon credits. All short sale orders are subject to the availability of the carbon credits being sold, which must be confirmed by our carbon credits loan department prior to the order being entered.

### Potential Uses of Short Selling:

Hedge current portfolio by short selling similar carbon credits or ETFs when you think the market may go down in the short term but don't want to sell the carbon credits you own to incur short-term capital gains.
Profit from the decline of a particular carbon credit, an entire industry, or the overall market.
Extend a bearish position when in-the-money calls you've written are exercised.

### Example of a Short Sale

short_selling
1. Seller shorts carbon credits at price A. A broker finds shares that can be borrowed for delivery.
2. Three trading days later, on settlement date, a broker provides shares for delivery. Seller then pays a variable interest rate on loan of shares for as long as the short position is maintained.
3. Seller enters a buy to cover order at price B. If the price is above the price at which it was originally sold short (B1), the short seller generally realizes a loss. If it is below the original selling price (B2), the short seller generally realizes a profit.*
Note that other factors may have an impact on profit or loss of the trade.

By implementing a carbon credit trading market based on the Blockchain designs discussed herein, as well as incorporating the carbon credit trading market into the IoT Platform implementation itself, the automation of carbon credit generation and monetization is possible. All aspects of such a system should conform to the ISO standards mentioned herein, as well as potentially follow the guidelines provided by the American Carbon Registry.

Many aspects of the blockchain design are desirable for a commodity exchange and/or trading platform. However, a blockchain-based architecture isn't necessarily required to implement a carbon credit or expanded commodity exchange. Either form should support the notion of immediate buy/sell transactions, options, forwards and/or futures, and swaps.

1. A method of securely tracking atmospheric emissions, comprising:
maintaining a secure chain of data blocks at a given computing node in a distributed network of computing nodes, wherein each of the computing nodes maintains the secure chain of data blocks, and wherein the secure chain of data blocks maintained at each computing node comprises one or more data blocks that respectively represent one or more transactions associated with a carbon credit, allowance, offset, asset or greenhouse gas atmospheric emissions representation; and
adding at least one data block to the secure chain of data blocks maintained at the given computing node in response to a triggering event associated with the atmospheric emissions representation, wherein the triggering event is a function of at least one measurement relevant to the atmospheric emissions representation;
wherein the maintaining and adding are implemented by a computer system operatively coupled to a memory associated with the given computing node and connected in signal communication with Internet of Things (IoT) equipment.

2. The method of claim 1, further comprising:
monitoring electricity use, detecting greenhouse gases, or using sensor-based computing devices to automate the validation or verification of carbon credits per ISO 14064-6 or like standards; and
producing or issuing carbon credits or a token-based representation of carbon credits listable on a market or financial exchange for trading or monetization.

3. The method of claim 1, further comprising:
measuring renewable energy production, energy efficiency, or alternative greenhouse gas emissions reduction before and after implementation of an emissions improvement; and
using the before and after measurements to generate carbon credits or a token-based representation of carbon credits on a blockchain.

4. The method of claim 1, further comprising:
managing carbon credits or a token-based representation of carbon credits though generation, validation, verification, or monetization on a trading market or through non-market mechanisms, wherein stakeholders are compensated by fiat currency payment, credit issuance, or digital currency or token issuance.

5. The method of claim 1, further comprising:
calculating, generating, issuing, managing, and monetizing carbon credits or a token-based representation of carbon credits through non-market, voluntary, regulated or regional markets, or cap-and-trade markets; and
recording at least parts of the transaction or payment to stakeholders in an automated manner.

6. The method of claim 1, further comprising:
generating carbon credits by measuring electrical production of a vehicle's regenerative braking system,
wherein the energy produced by the braking system is measure in an ISO 14064-3 compliant verification or validation, and
wherein the calculated carbon credits are monetized on a carbon credit trading market or used by the stakeholder to offset other emissions.

7. The method of claim 1, wherein the computer system comprises a blockchain-based storage mechanism implemented on one Cloud implementation, or implemented across two or more Cloud networks in real-time so that each Cloud installation mirrors the other Cloud installation(s) in real-time.

8. The method of claim 1, wherein the computer system manages or provides a program to calibrate IoT monitoring equipment remotely, wherein records, certifications, validation and verification reports are stored in an immutable blockchain implementation.

9. The method of claim 1, wherein the computer system is implemented in rural areas containing large land holdings used for farming, ranching, or forestry, putting nations and landowners in position to monetize carbon credits based on carbon storage value to promote the preservation of a nation's land ownership, land stewardship, greenhouse gas emissions reductions, soil health, ecological diversity, water quality, or air quality.

10. The method of claim 1, further comprising:
allocating to greenhouse gas emitters a specified number of allowances representing tons of CO2e that they may legally emit;
banking for an entity that can reduce its emissions below the number of allowances received the credits and/or a token-based representation of the credits for future compliance; and
selling excess credits/allowances to other entities whose emissions exceed annual allowances, which may include emission reductions or offsets defined as acceptable emission units recognized by a registry.

11. The method of claim 1, further comprising:
maintaining a ledger comprising two categories of records: transactions and blocks,
wherein blocks hold batches of valid transactions that are hashed and encoded into a Merkle tree or some structure that allows for ordering, linkage, searching and/or traversing data in a pattern-based or functional manner, and each block may include the hash of the prior block in the blockchain, linking the two.

12. The method of claim 1, wherein the computer system utilizes a blockchain mechanism configured for registering users of the IoT implementation, as well as registering all the equipment necessary to implement the carbon credit generation and monitoring software platform, potentially in a Cloud-computer based environment, wherein the blockchain implementation is configured within a single Cloud-computing environment, or spanning across two or more Cloud-computing environments to increase security as well as availability and stability of the entire system, and wherein all transactions are recorded by the blockchain so that the entire IoT implementation benefits from the blockchain's inherent features.

13. The method of claim 1, wherein the computer system implements a carbon credit, and/or a token-based representation of carbon credits, trading market based on a blockchain, as well as incorporating the carbon credit, and/or a token-based representation of a carbon credit, trading market into the IoT platform implementation itself, the automation of carbon credit, and/or a token-based representation of a carbon credit, generation and monetization conforming to ISO 14064-6 standards or the guidelines provided by a regulatory group.

14. The method of claim 1, wherein the computer system utilizes aspects of blockchain design for a commodities exchange and/or trading platform, but where a blockchain-based architecture isn't necessarily required to implement a carbon credit, and/or a token-based representation of a carbon credit, and/or an expanded commodities exchange, but supports any combination of immediate buy/sell transactions, options, forwards and/or futures, and swaps of carbon credits or associated tokens, by integrating the process of carbon accounting and offsetting in a token that may reside on a public, permissioned blockchain network where ownership rights are transmitted and traded.

15. The method of claim 1, wherein the computer system comprises a carbon credit based blockchain that eliminates double claiming of carbon credits, where double claiming might otherwise occur if the environmental benefit of a specific unit greenhouse gas emissions reduction or removal is counted towards more than one national or sector-wide emissions reduction target, where accounting procedures at both the national and international level are in place to track emissions reductions sold to buyers towards meeting their targets, wherein any applicable emissions reductions can be added back in to the host country national inventory.

16. The method of claim 1, wherein the computer system comprises a carbon credit based blockchain that eliminates double issuance of carbon credits, where double issuance is an instance in which a specific unit is issued more than once for the same emissions reduction or removal, wherein this is avoided by having preventative program rules and oversight processes in place, including cancellation of units by one program prior to re-issuance by another.

17. The method of claim 1, wherein the computer system comprises a carbon credit based blockchain that eliminates double selling of carbon credits, where double selling is an instance in which a specific unit of greenhouse gas emission reduction or removals is sold to multiple buyers, wherein this is avoided by having program rules and oversight processes in place to prevent double issuance and double use.

18. The method of claim 1, wherein the computer system comprises a carbon credit based blockchain that eliminates double use of carbon credits, where double use is an instance in which a specific unit of greenhouse gas reduction or removal is owned by more than one entity at a given time, carbon credits and/or a token-based representation of carbon credits can be a unit of exchange for tradable, project-based carbon offsets, carbon credits, and/or a token-based representation of carbon credits, refer to both emission reductions and enhancements in sequestration, wherein the system can issue one carbon credit, and/or a token-based representation of carbon credits, for each metric ton of CO2e emission reductions or removals verified against ISO-14064-6 standards and methodologies.

19. The method of claim 1, wherein the computer system comprises a digital registry, which is an online platform that tracks ownership of offsets and/or houses a ledger of all greenhouse gas emissions reduction projects, offset issuances, cancelations and/or retirements in any combination, and may provide transparent access to project records.

20. The method of claim 1, wherein the computer system comprises a greenhouse gas regulated “cap-and-trade” and/or regulated program that places an overall limit on emissions allowed from a specified set of entities, and/or issue tradable emission allowances or rights to emit that these entities can use for compliance, wherein allowances, which typically authorize an entity to emit a ton of CO2e, can be auctioned or freely distributed to covered entities or other parties, wherein at the end of a program's compliance period, covered entities submit an allowance for every ton of CO2e they emitted during that period, wherein a covered entity in a cap-and-trade program is provided a plurality of options for achieving compliance comprising submitting offsets in lieu of allowances for compliance purposes, using emission allowances it has received or purchased, acquiring offsets, and/or reducing its own emissions.
